(19) United States
(12) Patent Application Publication
(10) Pub. No.: US 2024/0343781 A1
(43) Pub. Date: Oct. 17, 2024

(54) CHARACTERIZATION OF POTENT AND BROADLY NEUTRALIZING MONOCLONAL ANTIBODIES AGAINST SARS-COV-2, ITS VARIANTS, AND RELATED CORONAVIRUSES AND METHODS OF USE

(71) Applicant: The Trustees of Columbia University in the City of New York, New York, NY (US)

(72) Inventors: David D. HO, Bedford, NY (US); Yaoxing HUANG, Brooklyn, NY (US); Lihong LIU, Fort Lee, NJ (US); Manoj S. NAIR, Fair Lawn, NJ (US); Jian YU, New Providence, NJ (US); Sho IKETANI, New York, NY (US)

(21) Appl. No.: 18/291,530

(22) PCT Filed: Jul. 22, 2022
(86) PCT No.: PCT/US2022/074075

§ 371 Date: Jan. 23, 2024

Related U.S. Application Data
(60) Provisional application No. 63/225,193, filed on Jul. 23, 2021.
(60) Provisional application No. 63/271,627, filed on Oct. 25, 2021.
(60) Provisional application No. 63/334,007, filed on Apr. 22, 2022.

(51) Int. Cl. C07K 16/10; A61P 31/20

(52) U.S. Cl. CPC C07K 16/1003; A61P 31/20; C07K 2317/14; C07K 2317/21; C07K 2317/33; C07K 2317/565; C07K 2317/76; C07K 2317/92

(57) ABSTRACT

The subject matter described herein relates to engineered monoclonal antibodies, derived from antibodies isolated from human patients, neutralizing a SARS-CoV-2 virus, its variants, or related coronaviruses.

This application claims the benefit of and priority to U.S. Provisional Patent Application No. 63/225,193, filed on Jul. 23, 2021, U.S. Provisional Patent Application No. 63/271,627, filed on Oct. 25, 2021, and U.S. Provisional Patent Application No. 63/334,007, filed on Apr. 22, 2022, the contents of each of which are hereby incorporated by reference in their entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

COVID-19 is a contagious disease caused by the SARS-COV-2 virus. COVID-19 spreads when an infected person breathes out droplets containing virus particles, which are then taken up by other people through their eyes, noses, or mouth. Effective treatment and preventative methods are needed to combat the further spread of COVID-19.

Neutralizing antibodies are responsible for defending cells from disease-causing pathogens such as viruses. Neutralizing antibodies can be produced by the body as part of the immune response. Their production is triggered by both infections and vaccinations against infections and can result in lifelong immunity.

Neutralizing antibodies are produced by B-cells and function by binding to a protein on a pathogen's surface. The binding prevents the pathogen from entering a host cell. In enveloped viruses, neutralizing antibodies block the attachment of a virus to a host cell and then its entry into the host cell. In non-enveloped viruses, neutralizing antibodies can bind to the capsid protein, which is the protein shell that surrounds the genetic information within a virus. Once a pathogen is neutralized by a neutralizing antibody, it is then degraded by white blood cells and filtered by the spleen.

### SUMMARY OF THE INVENTION

In certain aspects, the subject matter described herein provides an engineered monoclonal antibody, or a functional fragment thereof, selectively binding and neutralizing a at least a portion of a virus, wherein the antibody, or functional fragment thereof, comprises an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1, at least 60%, 62%, 65%, 67%, 70%, 72%, 75%, 77%, 80%, 82%, 85%, 87%, 90%, 92%, 95%, 97%, 98%, 99% identical to SEQ ID NO: 2, at least 60%, 62%, 65%, 67%, 70%, 72%, 75%, 77%, 80%, 82%, 85%, 87%, 90%, 92%, 95%, 97%, 98%, 99% identical to SEQ ID NO: 3, at least 60%, 62%, 65%, 67%, 70%, 72%, 75%, 77%, 80%, 82%, 85%, 87%, 90%, 92%, 95%, 97%, 98%, 99% identical to SEQ ID NO: 4, at least 60%, 62%, 65%, 67%, 70%, 72%, 75%, 77%, 80%, 82%, 85%, 87%, 90%, 92%, 95%, 97%, 98%, 99% identical to SEQ ID NO: 5, at least 60%, 62%, 65%, 67%, 70%, 72%, 75%, 77%, 80%, 82%, 85%, 87%, 90%, 92%, 95%, 97%, 98%, 99% identical to SEQ ID NO: 6, at least 60%, 62%, 65%, 67%, 70%, 72%, 75%, 77%, 80%, 82%, 85%, 87%, 90%, 92%, 95%, 97%, 98%, 99% identical to SEQ ID NO: 7, at least 60%, 62%, 65%, 67%, 70%, 72%, 75%, 77%, 80%, 82%, 85%, 87%, 90%, 92%, 95%, 97%, 98%, 99% identical to SEQ ID NO: 8, at least 60%, 62%, 65%, 67%, 70%, 72%, 75%, 77%, 80%, 82%, 85%, 87%, 90%, 92%, 95%, 97%, 98%, 99% identical to SEQ ID NO: 9, at least 60%, 62%, 65%, 67%, 70%, 72%, 75%, 77%, 80%, 82%, 85%, 87%, 90%, 92%, 95%, 97%, 98%, 99% identical to SEQ ID NO: 10, at least 60%, 62%, 65%, 67%, 70%, 72%, 75%, 77%, 80%, 82%, 85%, 87%, 90%, 92%, 95%, 97%, 98%, 99% identical to SEQ ID NO: 11, at least 60%, 62%, 65%, 67%, 70%, 72%, 75%, 77%, 80%, 82%, 85%, 87%, 90%, 92%, 95%, 97%, 98%, 99% identical to SEQ ID NO: 12, at least 60%, 62%, 65%, 67%, 70%, 72%, 75%, 77%, 80%, 82%, 85%, 87%, 90%, 92%, 95%, 97%, 98%, 99% identical to SEQ ID NO: 13, or at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence identical to SEQ ID NO: 1 and a light chain comprising an amino acid sequence identical to SEQ ID NO: 2. In some embodiments, the antibody or functional fragment thereof comprises a heavy chain comprising an amino acid sequence identical to SEQ ID NO: 3 and a light chain comprising an amino acid sequence identical to SEQ ID NO: 4. In some embodiments, the antibody or functional fragment thereof comprises a heavy chain comprising an amino acid sequence identical to SEQ ID NO: 5 and a light chain comprising an amino acid sequence identical SEQ ID NO: 6. In some embodiments, the antibody or functional fragment thereof comprises a heavy chain comprising an amino acid sequence identical to SEQ ID NO: 7 and a light chain comprising an amino acid sequence identical to SEQ ID NO: 8. In some embodiments, the antibody or functional fragment thereof comprises a heavy chain comprising an amino acid sequence identical to SEQ ID NO: 9 and a light chain comprising an amino acid sequence identical to SEQ ID NO: 10. In some embodiments, the antibody or functional fragment thereof comprises a heavy chain comprising an amino acid sequence identical to SEQ ID NO: 11 and a light chain comprising an amino acid sequence identical to SEQ ID NO: 12. In some embodiments, the antibody or functional fragment thereof comprises a heavy chain comprising an amino acid sequence identical to SEQ ID NO: 13 and a light chain comprising an amino acid sequence identical to SEQ ID NO: 14.

In some embodiments, the antibody or functional fragment thereof comprises a heavy chain comprising an amino acid sequence comprising the complementarity-determining regions (CDRs) of SEQ ID NO: 1 and a light chain comprising an amino acid sequence comprising the complementarity-determining regions (CDRs) of SEQ ID NO: 2. In some embodiments, the antibody or functional fragment thereof comprises a heavy chain comprising an amino acid sequence comprising the complementarity-determining regions (CDRs) of SEQ ID NO: 3 and a light chain comprising an amino acid sequence comprising the complementarity-determining regions (CDRs) of SEQ ID NO: 4. In some embodiments, the antibody or functional fragment thereof comprises a heavy chain comprising an amino acid sequence comprising the complementarity-determining regions (CDRs) of SEQ ID NO: 5 and a light chain comprising an amino acid sequence comprising the complementarity-determining regions (CDRs) of SEQ ID NO: 6. In some embodiments, the antibody or functional fragment thereof comprises a heavy chain comprising an amino acid sequence comprising the complementarity-determining regions (CDRs) of SEQ ID NO: 7 and a light chain comprising an amino acid sequence comprising the complementarity-determining regions (CDRs) of SEQ ID NO: 8. In some embodiments, the antibody or functional fragment thereof comprises a heavy chain comprising an amino acid sequence comprising the complementarity-determining regions (CDRs) of SEQ ID NO: 9 and a light chain comprising an amino acid sequence comprising the complementarity-determining regions (CDRs) of SEQ ID NO: 10. In some embodiments, the antibody or functional fragment thereof comprises a heavy chain comprising an amino acid sequence comprising the complementarity-determining regions (CDRs) of SEQ ID NO: 11 and a light chain comprising an amino acid sequence comprising the complementarity-determining regions (CDRs) of SEQ ID NO: 12. In some embodiments, the antibody or functional fragment thereof comprises a heavy chain comprising an amino acid sequence comprising the complementarity-determining regions (CDRs) of SEQ ID NO: 13 and a light chain comprising an amino acid sequence comprising the complementarity-determining regions (CDRs) of SEQ ID NO: 14.

In some embodiments, the virus is a coronavirus. In some embodiments, the coronavirus is selected from the coronaviruses consisting of SARS-COV-2, SARS-COV, MERS-COV, 229E, NL63, OC43, BM-4831, BtKY72, Rf1, RmYN02, Rs4081, Yunnan2011, ZC45, As6526, Rs4237, Rs4081, and HKU1. In some embodiments, the coronavirus is a SARS-COV-2 virus. In some embodiments, the SARS-COV-2 virus is a wild-type strain. In some embodiments, the SARS-COV-2 virus is a SARS-COV-2 D614G strain. In some embodiments, the SARS-COV-2 virus is a WAI strain. In some embodiments, the SARS-CoV-2 virus is a B.1.1.7 strain. In some embodiments, the SARS-COV-2 virus is a B.1.351 strain. In some embodiments, the SARS-COV-2 virus is a P.1 strain. In some embodiments, the SARS-COV-2 virus is a B.1.526 strain. In some embodiments, the SARS-COV-2 virus is a B.1.429 strain. In some embodiments, the SARS-COV-2 virus is a B.1.617.1 strain. In some embodiments, the SARS-COV-2 virus is a B.1.1.1 strain. In some embodiments, the SARS-CoV-2 virus is a B.1.617.2 strain. In some embodiments, the SARS-COV-2 virus is a B.1.526-E484K strain. In some embodiments, the SARS-COV-2 virus is a V236P12 strain. In some embodiments, the coronavirus is a SARS-COV virus. In some embodiments, the coronavirus is a MERS-COV virus. In some embodiments, the coronavirus is a NL63 virus. In some embodiments, the coronavirus is a GD-Pangolin virus. In some embodiments, the coronavirus is a GX-Pangolin virus. In some embodiments, the coronavirus is a RaTG13 virus. In some embodiments, the coronavirus is a WIV1 virus. In some embodiments, the coronavirus is a SHC014 virus. In some embodiments, the coronavirus is a LYRa11 virus. In some embodiments, the coronavirus is a Rs7327 virus. In some embodiments, the coronavirus is a Rs4231 virus. In some embodiments, the coronavirus is a Rs4084 virus.

In certain aspects, the subject matter described herein provides a method for treating a subject having a viral infection or preventing a subject from developing a viral infection, the method comprising administering to the subject a therapeutically effective amount of a composition comprising an engineered monoclonal antibody or a functional fragment thereof.

In some embodiments, the antibody comprises an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1, at least 60%, 62%, 65%, 67%, 70%, 72%, 75%, 77%, 80%, 82%, 85%, 87%, 90%, 92%, 95%, 97%, 98%, 99% identical to SEQ ID NO: 2, at least 60%, 62%, 65%, 67%, 70%, 72%, 75%, 77%, 80%, 82%, 85%, 87%, 90%, 92%, 95%, 97%, 98%, 99% identical to SEQ ID NO: 3, at least 60%, 62%, 65%, 67%, 70%, 72%, 75%, 77%, 80%, 82%, 85%, 87%, 90%, 92%, 95%, 97%, 98%, 99% identical to SEQ ID NO: 4, at least 60%, 62%, 65%, 67%, 70%, 72%, 75%, 77%, 80%, 82%, 85%, 87%, 90%, 92%, 95%, 97%, 98%, 99% identical to SEQ ID NO: 5, at least 60%, 62%, 65%, 67%, 70%, 72%, 75%, 77%, 80%, 82%, 85%, 87%, 90%, 92%, 95%, 97%, 98%, 99% identical to SEQ ID NO: 6, at least 60%, 62%, 65%, 67%, 70%, 72%, 75%, 77%, 80%, 82%, 85%, 87%, 90%, 92%, 95%, 97%, 98%, 99% identical to SEQ ID NO: 7, at least 60%, 62%, 65%, 67%, 70%, 72%, 75%, 77%, 80%, 82%, 85%, 87%, 90%, 92%, 95%, 97%, 98%, 99% identical to SEQ ID NO: 8, at least 60%, 62%, 65%, 67%, 70%, 72%, 75%, 77%, 80%, 82%, 85%, 87%, 90%, 92%, 95%, 97%, 98%, 99% identical to SEQ ID NO: 9, at least 60%, 62%, 65%, 67%, 70%, 72%, 75%, 77%, 80%, 82%, 85%, 87%, 90%, 92%, 95%, 97%, 98%, 99% identical to SEQ ID NO: 10, at least 60%, 62%, 65%, 67%, 70%, 72%, 75%, 77%, 80%, 82%, 85%, 87%, 90%, 92%, 95%, 97%, 98%, 99% identical to SEQ ID NO: 11, at least 60%, 62%, 65%, 67%, 70%, 72%, 75%, 77%, 80%, 82%, 85%, 87%, 90%, 92%, 95%, 97%, 98%, 99% identical to SEQ ID NO: 12, at least 60%, 62%, 65%, 67%, 70%, 72%, 75%, 77%, 80%, 82%, 85%, 87%, 90%, 92%, 95%, 97%, 98%, 99% identical to SEQ ID NO: 13, or at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence identical to SEQ ID NO: 1 and a light chain comprising an amino acid sequence identical to SEQ ID NO: 2. In some embodiments, the antibody or functional fragment thereof comprises a heavy chain comprising an amino acid sequence identical to SEQ ID NO: 3 and a light chain comprising an amino acid sequence identical to SEQ ID NO: 4. In some embodiments, the antibody or functional fragment thereof comprises a heavy chain comprising an amino acid sequence identical to SEQ ID NO: 5 and a light chain comprising an amino acid sequence identical to SEQ ID NO: 6. In some embodiments, the antibody or functional fragment thereof comprises a heavy chain comprising an amino acid sequence identical to SEQ ID NO: 7 and a light chain comprising an amino acid sequence identical to SEQ ID NO: 8. In some embodiments, the antibody or functional fragment thereof comprises a heavy chain comprising an amino acid sequence identical to SEQ ID NO: 9 and a light chain comprising an amino acid sequence identical to SEQ ID NO: 10. In some embodiments, the antibody or functional fragment thereof comprises a heavy chain comprising an amino acid sequence identical to SEQ ID NO: 11 and a light chain comprising an amino acid sequence identical to SEQ ID NO: 12. In some embodiments, the antibody or functional fragment thereof comprises a heavy chain comprising an amino acid sequence identical to SEQ ID NO: 13 and a light chain comprising an amino acid sequence identical to SEQ ID NO: 14.

In some embodiments, the antibody or functional fragment thereof comprises a heavy chain comprising an amino acid sequence comprising the complementarity-determining regions (CDRs) of SEQ ID NO: 1 and a light chain comprising an amino acid sequence comprising the complementarity-determining regions (CDRs) of SEQ ID NO: 2. In some embodiments, the antibody or functional fragment thereof comprises a heavy chain comprising an amino acid sequence comprising the complementarity-determining regions (CDRs) of SEQ ID NO: 3 and a light chain comprising an amino acid sequence comprising the complementarity-determining regions (CDRs) of SEQ ID NO: 4. In some embodiments, the antibody or functional fragment thereof comprises a heavy chain comprising an amino acid sequence comprising the complementarity-determining regions (CDRs) of SEQ ID NO: 5 and a light chain comprising an amino acid sequence comprising the complementarity-determining regions (CDRs) of SEQ ID NO: 6. In some embodiments, the antibody or functional fragment thereof comprises a heavy chain comprising an amino acid sequence comprising the complementarity-determining regions (CDRs) of SEQ ID NO: 7 and a light chain comprising an amino acid sequence comprising the complementarity-determining regions (CDRs) of SEQ ID NO: 8. In some embodiments, the antibody or functional fragment thereof comprises a heavy chain comprising an amino acid sequence comprising the complementarity-determining regions (CDRs) of SEQ ID NO: 9 and a light chain comprising an amino acid sequence comprising the complementarity-determining regions (CDRs) of SEQ ID NO: 10. In some embodiments, the antibody or functional fragment thereof comprises a heavy chain comprising an amino acid sequence comprising the complementarity-determining regions (CDRs) of SEQ ID NO: 11 and a light chain comprising an amino acid sequence comprising the complementarity-determining regions (CDRs) of SEQ ID NO: 12. In some embodiments, the antibody or functional fragment thereof comprises a heavy chain comprising an amino acid sequence comprising the complementarity-determining regions (CDRs) of SEQ ID NO: 13 and a light chain comprising an amino acid sequence comprising the complementarity-determining regions (CDRs) of SEQ ID NO: 14.

In some embodiments, the virus is a coronavirus. In some embodiments, the coronavirus is selected from the strains consisting of SARS-COV-2, SARS-COV, MERS-CoV, 229E, NL63, OC43, BM-4831, BtKY72, Rf1, RmYN02, Rs4081, Yunnan2011, ZC45, As6526, Rs4237, Rs4081, and HKU1. In some embodiments, the coronavirus is a SARS-CoV-2 virus. In some embodiments, the SARS-COV-2 virus is a wild-type strain. In some embodiments, the SARS-COV-2 virus is a SARS-COV-2 D614G strain. In some embodiments, the SARS-COV-2 virus is a WAI strain. In some embodiments, the SARS-CoV-2 virus is a B.1.1.7 strain. In some embodiments, the SARS-COV-2 virus is a B.1.351 strain. In some embodiments, the SARS-COV-2 virus is a P.1 strain. In some embodiments, the SARS-COV-2 virus is a B.1.526 strain. In some embodiments, the SARS-COV-2 virus is a B.1.429 strain. In some embodiments, the SARS-COV-2 virus is a B.1.617.1 strain. In some embodiments, the SARS-COV-2 virus is a B.1.1.1 strain. In some embodiments, the SARS-CoV-2 virus is a B.1.617.2 strain. In some embodiments, the SARS-COV-2 virus is a B.1.526-E484K strain. In some embodiments, the SARS-COV-2 virus is a V236P12 strain. In some embodiments, the coronavirus is a SARS-COV virus. In some embodiments, the coronavirus is a MERS-COV virus. In some embodiments, the coronavirus is a NL63 virus. In some embodiments, the coronavirus is a GD-Pangolin virus. In some embodiments, the coronavirus is a GX-Pangolin virus. In some embodiments, the coronavirus is a RaTG13 virus. In some embodiments, the coronavirus is a WIV1 virus. In some embodiments, the coronavirus is a SHC014 virus. In some embodiments, the coronavirus is a LYRa11 virus. In some embodiments, the coronavirus is a Rs7327 virus. In some embodiments, the coronavirus is a Rs4231 virus. In some embodiments, the coronavirus is a Rs4084 virus.

In some embodiments, the composition comprises a cocktail of two or more engineered monoclonal antibodies. In some embodiments, the composition further comprises an antiviral agent.

In certain aspects, the subject matter described herein provides a method of producing engineered monoclonal antibodies, the method comprising: a) isolating a biological sample from a subject infected with a virus; b) determining whether the biological sample has neutralizing activity against the virus; c) isolating peripheral blood mononuclear cells (PMBCs) from the subjects whose biological sample has neutralizing activity in step b); d) isolating coronavirus S protein trimer-specific memory single B cells from the PMBCs in c); e) recovering one of more monoclonal antibody sequences selective for a coronavirus S protein trimer from the B cells; f) determining the sequence encoding the one or more monoclonal antibodies recovered from the single B cells; g) cloning the sequence encoding the one or more monoclonal antibodies into one or more expression vectors; h) expressing one or more engineered monoclonal antibody in vitro; and i) purifying the one or more engineered monoclonal antibody.

In some embodiments, the biological sample is plasma. In some embodiments, the subject is not vaccinated against the virus. In some embodiments, the subject is vaccinated against the virus. In some embodiments, the determining in f) comprises high throughput sequencing.

In some embodiments, the virus is a coronavirus. In some embodiments, the coronavirus is selected from the strains consisting of SARS-COV-2, SARS-COV, MERS-CoV, 229E, NL63, OC43, BM-4831, BtKY72, Rf1, RmYN02, Rs4081, Yunnan2011, ZC45, As6526, Rs4237, Rs4081, and HKU1. In some embodiments, the coronavirus is a SARS-CoV-2 virus. In some embodiments, the SARS-COV-2 virus is a wild-type strain. In some embodiments, the SARS-COV-2 virus is a SARS-COV-2 D614G strain. In some embodiments, the SARS-COV-2 virus is a WA1 strain. In some embodiments, the SARS-CoV-2 virus is a B.1.1.7 strain. In some embodiments, the SARS-COV-2 virus is a B.1.351 strain. In some embodiments, the SARS-COV-2 virus is a P.1 strain. In some embodiments, the SARS-COV-2 virus is a B.1.526 strain. In some embodiments, the SARS-COV-2 virus is a B.1.429 strain. In some embodiments, the SARS-COV-2 virus is a B.1.617.1 strain. In some embodiments, the SARS-COV-2 virus is a B.1.1.1 strain. In some embodiments, the SARS-CoV-2 virus is a B.1.617.2 strain. In some embodiments, the SARS-COV-2 virus is a B.1.526-E484K strain. In some embodiments, the SARS-COV-2 virus is a V236P12 strain. In some embodiments, the coronavirus is a SARS-COV virus. In some embodiments, the coronavirus is a MERS-COV virus. In some embodiments, the coronavirus is a NL63 virus. In some embodiments, the coronavirus is a GD-Pangolin virus. In some embodiments, the coronavirus is a GX-Pangolin virus. In some embodiments, the coronavirus is a RaTG13 virus. In some embodiments, the coronavirus is a WIV1 virus. In some embodiments, the coronavirus is a SHC014 virus. In some embodiments, the coronavirus is a LYRa11 virus. In some embodiments, the coronavirus is a Rs7327 virus. In some embodiments, the coronavirus is a Rs4231 virus. In some embodiments, the coronavirus is a Rs4084 virus.

In certain aspects, the subject matter described herein provides an engineered monoclonal antibody, or a functional fragment thereof, wherein the antibody or fragment thereof selectively binds at least a portion of a virus.

In some embodiments, the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 17. In some embodiments, the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence identical to SEQ ID NO: 15 and a light chain comprising an amino acid sequence identical to SEQ ID NO: 16. In some embodiments, the antibody or functional fragment thereof comprises a heavy chain comprising an amino acid sequence identical to SEQ ID NO: 17 and a light chain comprising an amino acid sequence identical to SEQ ID NO: 18. In some embodiments, the antibody or functional fragment thereof comprises a heavy chain comprising an amino acid sequence comprising the complementarity-determining regions (CDRs) of SEQ ID NO: 15 and a light chain comprising an amino acid sequence comprising the complementarity-determining regions (CDRs) of SEQ ID NO: 16. In some embodiments, the antibody or functional fragment thereof comprises a heavy chain comprising an amino acid sequence comprising the complementarity-determining regions (CDRs) of SEQ ID NO: 17 and a light chain comprising an amino acid sequence comprising the complementarity-determining regions (CDRs) of SEQ ID NO: 18.

In some embodiments, the virus is a coronavirus. In some embodiments, the coronavirus is a coronavirus variant. In some embodiments, the coronavirus is a coronavirus-related virus. In some embodiments, the coronavirus is a wildtype SARS-COV-2 (D614G) virus. In some embodiments, the coronavirus is a SARS-COV-2 B.1.1.7 variant virus. In some embodiments, the coronavirus is a SARS-COV-2 B.1.351 variant virus. In some embodiments, the coronavirus is a SARS-COV-2 P.1 variant virus. In some embodiments, the coronavirus is a SARS-COV-2 B.1.526 variant virus. In some embodiments, the coronavirus is a SARS-COV-2 B.1.429 variant virus. In some embodiments, the coronavirus is a SARS-CoV-2 B.1.617.1v1 variant virus. In some embodiments, the coronavirus is a SARS-COV-2 B.1.617.2 variant virus. In some embodiments, the coronavirus is a SARS-COV-2 B.1.1.1 variant virus. In some embodiments, the coronavirus is a SARS-COV-2 C.37 variant virus. In some embodiments, the coronavirus is a SARS-COV-2 Omicron (B.1.1.529) variant virus. In some embodiments, the coronavirus is a SARS-COV-2 Omicron BA.1 (B.1.1.529.1) variant virus. In some embodiments, the coronavirus is a SARS-COV-2 Omicron BA.1.1 (B.1.1.529.1.1) variant virus. In some embodiments, the coronavirus is a SARS-COV-2 Omicron BA.2 (B.1.1.529.2) variant virus. In some embodiments, the coronavirus is a SARS-CoV-2 B.1.621 variant virus. In some embodiments, the coronavirus is a SARS-COV-2 WA1 virus. In some embodiments, the coronavirus is any virus of Table 1. In some embodiments, the coronavirus is a SARS-COV virus.

In some embodiments, the at least a portion of a virus comprises a coronavirus spike protein. In some embodiments, the at least a portion of a virus comprises a juncture between a subdomain 1 (SD1) and a N-Terminal Domain (NTD) of a coronavirus spike protein. In some embodiments, the at least a portion of a virus comprises a subdomain 1 (SD1) of a coronavirus spike protein. In some embodiments, the at least a portion of a virus comprises a N-Terminal Domain (NTD) of a coronavirus spike protein.

In certain aspects, the subject matter described herein provides a method for treating a subject having a viral infection or preventing a subject from developing a viral infection, the method comprising administering to the subject a therapeutically effective amount of a composition comprising an engineered monoclonal antibody or a functional fragment thereof, wherein the antibody or fragment thereof selectively binds at least a portion of the virus.

In some embodiments, the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 17. In some embodiments, the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence identical to SEQ ID NO: 15 and a light chain comprising an amino acid sequence identical to SEQ ID NO: 16. In some embodiments, the antibody or functional fragment thereof comprises a heavy chain comprising an amino acid sequence identical to SEQ ID NO: 17 and a light chain comprising an amino acid sequence identical to SEQ ID NO: 18.

In some embodiments, the antibody or functional fragment thereof comprises a heavy chain comprising an amino acid sequence comprising the complementarity-determining regions (CDRs) of SEQ ID NO: 15 and a light chain comprising an amino acid sequence comprising the complementarity-determining regions (CDRs) of SEQ ID NO: 16. In some embodiments, the antibody or functional fragment thereof comprises a heavy chain comprising an amino acid sequence comprising the complementarity-determining regions (CDRs) of SEQ ID NO: 17 and a light chain comprising an amino acid sequence comprising the complementarity-determining regions (CDRs) of SEQ ID NO: 18.

In some embodiments, the virus is a coronavirus. In some embodiments, the coronavirus is a coronavirus variant. In some embodiments, the coronavirus is a coronavirus-related virus. In some embodiments, the coronavirus is a wildtype SARS-COV-2 (D614G) virus. In some embodiments, the coronavirus is a SARS-COV-2 B.1.1.7 variant virus. In some embodiments, the coronavirus is a SARS-COV-2 B.1.351 variant virus. In some embodiments, the coronavirus is a SARS-COV-2 P.1 variant virus. In some embodiments, the coronavirus is a SARS-COV-2 B.1.526 variant virus. In some embodiments, the coronavirus is a SARS-COV-2 B.1.429 variant virus. In some embodiments, the coronavirus is a SARS-CoV-2 B.1.617.1v1 variant virus. In some embodiments, the coronavirus is a SARS-COV-2 B.1.617.2 variant virus. In some embodiments, the coronavirus is a SARS-COV-2 B.1.1.1 variant virus. In some embodiments, the coronavirus is a SARS-COV-2 D614G virus. In some embodiments, the coronavirus is a SARS-COV-2 C.37 variant virus. In some embodiments, the coronavirus is a SARS-COV-2 Omicron (B.1.1.529) variant virus. In some embodiments, the coronavirus is a SARS-COV-2 Omicron BA.1 (B.1.1.529.1) variant virus. In some embodiments, the coronavirus is a SARS-COV-2 Omicron BA.1.1 (B.1.1.529.1.1) variant virus. In some embodiments, the coronavirus is a SARS-COV-2 Omicron BA.2 (B.1.1.529.2) variant virus. In some embodiments, the coronavirus is a SARS-COV-2 B.1.621 variant virus. In some embodiments, the coronavirus is a SARS-COV-2 WA1 virus. In some embodiments, the coronavirus is any virus of Table 1. In some embodiments, the coronavirus is a SARS-CoV virus.

In some embodiments, the at least a portion of a virus comprises a coronavirus spike protein. In some embodiments, the at least a portion of a virus comprises a juncture between a subdomain 1 (SD1) and a N-Terminal Domain (NTD) of a coronavirus spike protein. In some embodiments, the at least a portion of a virus comprises a subdomain 1 (SD1) of a coronavirus spike protein. In some embodiments, the at least a portion of a virus comprises a N-Terminal Domain (NTD) of a coronavirus spike protein.

In certain aspects, the subject matter described herein provides a pharmaceutical composition comprising an engineered monoclonal antibody or a functional fragment thereof, wherein the antibody or fragment thereof selectively binds at least a portion of a virus.

In some embodiments, the antibody or fragment thereof comprises an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 1, at least 60%, 62%, 65%, 67%, 70%, 72%, 75%, 77%, 80%, 82%, 85%, 87%, 90%, 92%, 95%, 97%, 98%, 99% identical to SEQ ID NO: 2, at least 60%, 62%, 65%, 67%, 70%, 72%, 75%, 77%, 80%, 82%, 85%, 87%, 90%, 92%, 95%, 97%, 98%, 99% identical to SEQ ID NO: 3, at least 60%, 62%, 65%, 67%, 70%, 72%, 75%, 77%, 80%, 82%, 85%, 87%, 90%, 92%, 95%, 97%, 98%, 99% identical to SEQ ID NO: 4, at least 60%, 62%, 65%, 67%, 70%, 72%, 75%, 77%, 80%, 82%, 85%, 87%, 90%, 92%, 95%, 97%, 98%, 99% identical to SEQ ID NO: 5, at least 60%, 62%, 65%, 67%, 70%, 72%, 75%, 77%, 80%, 82%, 85%, 87%, 90%, 92%, 95%, 97%, 98%, 99% identical to SEQ ID NO: 6, at least 60%, 62%, 65%, 67%, 70%, 72%, 75%, 77%, 80%, 82%, 85%, 87%, 90%, 92%, 95%, 97%, 98%, 99% identical to SEQ ID NO: 7, at least 60%, 62%, 65%, 67%, 70%, 72%, 75%, 77%, 80%, 82%, 85%, 87%, 90%, 92%, 95%, 97%, 98%, 99% identical to SEQ ID NO: 8, at least 60%, 62%, 65%, 67%, 70%, 72%, 75%, 77%, 80%, 82%, 85%, 87%, 90%, 92%, 95%, 97%, 98%, 99% identical to SEQ ID NO: 9, at least 60%, 62%, 65%, 67%, 70%, 72%, 75%, 77%, 80%, 82%, 85%, 87%, 90%, 92%, 95%, 97%, 98%, 99% identical to SEQ ID NO: 10, at least 60%, 62%, 65%, 67%, 70%, 72%, 75%, 77%, 80%, 82%, 85%, 87%, 90%, 92%, 95%, 97%, 98%, 99% identical to SEQ ID NO: 11, at least 60%, 62%, 65%, 67%, 70%, 72%, 75%, 77%, 80%, 82%, 85%, 87%, 90%, 92%, 95%, 97%, 98%, 99% identical to SEQ ID NO: 12, at least 60%, 62%, 65%, 67%, 70%, 72%, 75%, 77%, 80%, 82%, 85%, 87%, 90%, 92%, 95%, 97%, 98%, 99% identical to SEQ ID NO: 13, at least 60%, 62%, 65%, 67%, 70%, 72%, 75%, 77%, 80%, 82%, 85%, 87%, 90%, 92%, 95%, 97%, 98%, 99% identical to SEQ ID NO: 14, at least 60%, 62%, 65%, 67%, 70%, 72%, 75%, 77%, 80%, 82%, 85%, 87%, 90%, 92%, 95%, 97%, 98%, 99% identical to SEQ ID NO: 15, at least 60%, 62%, 65%, 67%, 70%, 72%, 75%, 77%, 80%, 82%, 85%, 87%, 90%, 92%, 95%, 97%, 98%, 99% identical to SEQ ID NO: 16, at least 60%, 62%, 65%, 67%, 70%, 72%, 75%, 77%, 80%, 82%, 85%, 87%, 90%, 92%, 95%, 97%, 98%, 99% identical to SEQ ID NO: 17, or at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence identical to SEQ ID NO: 1 and a light chain comprising an amino acid sequence identical to SEQ ID NO: 2. In some embodiments, the antibody or functional fragment thereof comprises a heavy chain comprising an amino acid sequence identical to SEQ ID NO: 3 and a light chain comprising an amino acid sequence identical to SEQ ID NO: 4. In some embodiments, the antibody or functional fragment thereof comprises a heavy chain comprising an amino acid sequence identical to SEQ ID NO: 5 and a light chain comprising an amino acid sequence identical to SEQ ID NO: 6. In some embodiments, the antibody or functional fragment thereof comprises a heavy chain comprising an amino acid sequence identical to SEQ ID NO: 7 and a light chain comprising an amino acid sequence identical to SEQ ID NO: 8. In some embodiments, the antibody or functional fragment thereof comprises a heavy chain comprising an amino acid sequence identical to SEQ ID NO: 9 and a light chain comprising an amino acid sequence identical to SEQ ID NO: 10. In some embodiments, the antibody or functional fragment thereof comprises a heavy chain comprising an amino acid sequence identical to SEQ ID NO: 11 and a light chain comprising an amino acid sequence identical to SEQ ID NO: 12. In some embodiments, the antibody or functional fragment thereof comprises a heavy chain comprising an amino acid sequence identical to SEQ ID NO: 13 and a light chain comprising an amino acid sequence identical to SEQ ID NO: 14. In some embodiments, the antibody or functional fragment thereof comprises a heavy chain comprising an amino acid sequence identical to SEQ ID NO: 15 and a light chain comprising an amino acid sequence identical to SEQ ID NO: 16. In some embodiments, the antibody or functional fragment thereof comprises a heavy chain comprising an amino acid sequence identical to SEQ ID NO: 17 and a light chain comprising an amino acid sequence identical to SEQ ID NO: 18.

In some embodiments, the antibody or functional fragment thereof comprises a heavy chain comprising an amino acid sequence comprising the complementarity-determining regions (CDRs) of SEQ ID NO: 1 and a light chain comprising an amino acid sequence comprising the complementarity-determining regions (CDRs) of SEQ ID NO: 2. In some embodiments, the antibody or functional fragment thereof comprises a heavy chain comprising an amino acid sequence comprising the complementarity-determining regions (CDRs) of SEQ ID NO: 3 and a light chain comprising an amino acid sequence comprising the complementarity-determining regions (CDRs) of SEQ ID NO: 4. In some embodiments, the antibody or functional fragment thereof comprises a heavy chain comprising an amino acid sequence comprising the complementarity-determining regions (CDRs) of SEQ ID NO: 5 and a light chain comprising an amino acid sequence comprising the complementarity-determining regions (CDRs) of SEQ ID NO: 6. In some embodiments, the antibody or functional fragment thereof comprises a heavy chain comprising an amino acid sequence comprising the complementarity-determining regions (CDRs) of SEQ ID NO: 7 and a light chain comprising an amino acid sequence comprising the complementarity-determining regions (CDRs) of SEQ ID NO: 8. In some embodiments, the antibody or functional fragment thereof comprises a heavy chain comprising an amino acid sequence comprising the complementarity-determining regions (CDRs) of SEQ ID NO: 9 and a light chain comprising an amino acid sequence comprising the complementarity-determining regions (CDRs) of SEQ ID NO: 10. In some embodiments, the antibody or functional fragment thereof comprises a heavy chain comprising an amino acid sequence comprising the complementarity-determining regions (CDRs) of SEQ ID NO: 11 and a light chain comprising an amino acid sequence comprising the complementarity-determining regions (CDRs) of SEQ ID NO: 12. In some embodiments, the antibody or functional fragment thereof comprises a heavy chain comprising an amino acid sequence comprising the complementarity-determining regions (CDRs) of SEQ ID NO: 13 and a light chain comprising an amino acid sequence comprising the complementarity-determining regions (CDRs) of SEQ ID NO: 14. In some embodiments, the antibody or functional fragment thereof comprises a heavy chain comprising an amino acid sequence comprising the complementarity-determining regions (CDRs) of SEQ ID NO: 15 and a light chain comprising an amino acid sequence comprising the complementarity-determining regions (CDRs) of SEQ ID NO: 16. In some embodiments, the antibody or functional fragment thereof comprises a heavy chain comprising an amino acid sequence comprising the complementarity-determining regions (CDRs) of SEQ ID NO: 17 and a light chain comprising an amino acid sequence comprising the complementarity-determining regions (CDRs) of SEQ ID NO: 18.

In some embodiments, the virus is a coronavirus. In some embodiments, the coronavirus is a coronavirus variant. In some embodiments, the coronavirus is a coronavirus-related virus. In some embodiments, the coronavirus is a wildtype SARS-COV-2 (D614G) virus. In some embodiments, the coronavirus is a SARS-COV-2 B.1.1.7 variant virus. In some embodiments, the coronavirus is a SARS-COV-2 B.1.351 variant virus. In some embodiments, the coronavirus is a SARS-COV-2 P.1 variant virus. In some embodiments, the coronavirus is a SARS-COV-2 B.1.526 variant virus. In some embodiments, the coronavirus is a SARS-COV-2 B.1.429 variant virus. In some embodiments, the coronavirus is a SARS-CoV-2 B.1.617.1v1 variant virus. In some embodiments, the coronavirus is a SARS-COV-2 B.1.617.2 variant virus. In some embodiments, the coronavirus is a SARS-COV-2 B.1.1.1 variant virus. In some embodiments, the coronavirus is a SARS-COV-2 D614G virus. In some embodiments, the coronavirus is a SARS-COV-2 C.37 variant virus. In some embodiments, the coronavirus is a SARS-COV-2 Omicron (B.1.1.529) variant virus. In some embodiments, the coronavirus is a SARS-COV-2 Omicron BA.1 (B.1.1.529.1) variant virus. In some embodiments, the coronavirus is a SARS-COV-2 Omicron BA.1.1 (B.1.1.529.1.1) variant virus. In some embodiments, the coronavirus is a SARS-COV-2 Omicron BA.2 (B.1.1.529.2) variant virus. In some embodiments, the coronavirus is a SARS-COV-2 B.1.621 variant virus. In some embodiments, the coronavirus is a SARS-COV-2 WA1 virus. In some embodiments, the coronavirus is any virus of Table 1. In some embodiments, the coronavirus is a SARS-CoV virus.

In some embodiments, the at least a portion of a virus comprises a coronavirus spike protein. In some embodiments, the at least a portion of a virus comprises a juncture between a subdomain 1 (SD1) and a N-Terminal Domain (NTD) of a coronavirus spike protein. In some embodiments, the at least a portion of a virus comprises a subdomain 1 (SD1) of a coronavirus spike protein. In some embodiments, the at least a portion of a virus comprises a N-Terminal Domain (NTD) of a coronavirus spike protein.

In certain aspects the subject matter described herein provides a polynucleotide encoding an antibody or a fragment thereof, wherein the antibody or fragment thereof comprises any one of SEQ ID NOs: 1-18.

In certain aspects the subject matter described herein provides a vector comprising a polynucleotide encoding an antibody or a fragment thereof, wherein the antibody or fragment thereof comprises any one of SEQ ID NOs: 1-18.

### BRIEF DESCRIPTION OF THE FIGURES

The patent or application file contains at least one drawing in color. To conform to the requirements for PCT patent applications, many of the figures presented herein are black and white representations of images originally created in color.

FIGS. 1A-C show SARS-COV-2 Spike trimer-specific antibody isolation. FIG. 1A shows an illustrative representation of SARS-COV-2 S trimer-specific antibody isolation strategy. FIG. 1B shows sorting of various cell types from COVID-19 patients and a healthy donor. FIG. 1C shows side scatter area (SSC-A) flow cytometry using the Spike trimer of the SARS-COV-2 virus.

FIGS. 2A-L show neutralization profiles of down-selected monoclonal antibodies (mAbs) on wild-type (WT) and variant of concern (VOC) pseudoviruses (PVs). FIG. 2A shows neutralization profiles of mAbs REGN10933 and REGN10987. FIG. 2B shows neutralization profiles of mAb 2-7. FIG. 2C shows neutralization profiles of mAb 2-36. FIG. 2D shows neutralization profiles of mAbs LY-COV555 and CB6. FIG. 2E shows neutralization profiles of mAb Brii-196 and Brii-198. FIG. 2F shows neutralization profiles of mAbs COV2-2196 and COV2-2130. FIG. 2G shows neutralization profiles of engineered monoclonal antibody 10-16. FIG. 2H shows neutralization profiles of engineered monoclonal antibody 10-40. FIG. 2I shows neutralization profiles of engineered monoclonal antibody 11-11. FIG. 2J shows neutralization profiles of engineered monoclonal antibody 12-6. FIG. 2K shows neutralization profiles of engineered monoclonal antibody 12-27. FIG. 2L shows neutralization profiles of engineered monoclonal antibody 13-2.

FIG. 3 shows the half maximal inhibitory concentration (IC₅₀) values from neutralization of SARS-COV-2 virus, its variants, and SARS-COV pseudoviruses with down-selected engineered monoclonal antibodies disclosed herein.

FIGS. 4A-J show neutralization profiles of authentic SARS-COV-2 virus and 6 variants. FIG. 4A shows neutralization profiles with engineered mAb 2-7. FIG. 4B shows neutralization profiles with mAb S309. FIG. 4C shows neutralization profiles with mAb 2-36. FIG. 4D shows neutralization profiles with engineered monoclonal antibody 10-16. FIG. 4E shows neutralization profiles with engineered monoclonal antibody 10-40. FIG. 4F shows neutralization profiles with engineered monoclonal antibody 11-11. FIG. 4G shows neutralization profiles with engineered monoclonal antibody 12-27. FIG. 4H shows neutralization profiles with engineered monoclonal antibody 12-6. FIG. 4I shows neutralization with engineered monoclonal antibody 13-2. FIG. 4J shows potency with IC₅₀ of various monoclonal antibodies disclosed herein.

FIGS. 5A-H show the breadth and potency of engineered mAbs 10-28, 10-40 and 11-11 against SARS-like coronaviruses, which use the human angiotensin-converting enzyme 2 (ACE2) receptor to enter the host cell, in comparison with other mAbs. FIG. 5A shows neutralization profiles for monoclonal antibody 2-36. FIG. 5B shows neutralization profiles for monoclonal antibody S309. FIG. 5C shows neutralization profiles for monoclonal antibody COVA1-16. FIG. 5D shows neutralization profiles for monoclonal antibody CR3022. FIG. 5E shows neutralization profiles for engineered monoclonal antibody 10-28. FIG. 5F shows neutralization profiles for engineered monoclonal antibody 10-40. FIG. 5G shows neutralization profiles for engineered monoclonal antibody 11-11. FIG. 5H shows potency with IC₅₀ of various monoclonal antibodies disclosed herein.

FIGS. 6A-C show identification of three broadly neutralizing sarbecovirus (the viral subgenus containing SARS-COV and SARS-COV-2 viruses)-specific engineered monoclonal antibodies-mAbs 10-28, 10-40, and 11-11. FIG. 6A shows that the engineered antibodies were tested for binding to the spike trimers of SARS-COV-2 D614G, B.1.351, or SARS-COV, as well as the receptor binding domain (RBD) of SARS-COV-2 D614G by ELISA. Data are shown as mean±SD of two technical replicates. FIG. 6B shows inhibition of ACE2 binding to SARS-COV-2 D614G, B.1.351, and SARS-COV spike trimers by engineered monoclonal antibodies 10-28, 10-40, and 11-11. Testing was performed by ELISA. Data are shown as mean+SD of two technical replicates. FIG. 6C shows epitope mapping by competition ELISA of RBD-directed broadly neutralizing antibodies. Select antibodies were tested for blocking binding of biotinylated monoclonal antibodies to SARS-CoV-2 D614G spike trimer.

FIGS. 7A-E show breadth and potency of engineered monoclonal antibodies 10-40, 10-28, and 11-11. FIG. 7A shows neutralization titers (IC₅₀) of selected monoclonal antibodies against VSV pseudotypes with SARS-COV-2 D614G and variant spikes. FIG. 7B shows neutralization titers (IC₅₀) of selected monoclonal antibodies against authentic SARS-CoV-2 and SARS-COV strains in a cytopathic effect reduction assay. FIG. 7C shows neutralization titers (IC₅₀) of selected monoclonal antibodies against VSV pseudotypes with sarbecovirus spikes. FIG. 7D shows binding titers (EC₅₀) of selected monoclonal antibodies to purified RBD proteins, representative of the different sarbecovirus clades, and SARS-CoV-2 variants as measured by ELISA. FIG. 7E shows antibody binding to coronavirus spike proteins expressed on the cell surface. GX-Pangolin, RaTG13, and GD-Pangolin are SARS-CoV-2 related viruses and the rest are non-human ACE2 dependent viruses.

FIGS. 8A-F show structural and bioinformatic analyses of isolated antibodies 10-40, 10-28, and 11-11. FIG. 8A shows Cryo-EM reconstructions and recognition footprints for monoclonal antibodies 10-40, 10-28, and 11-11 Fabs bound to the B.1.351 spike protein. The spike is colored in light gray, with the receptor binding domains (RBDs) in green with the glycans in purple, oriented with the membrane towards the bottom. The 10-40 Fabs are colored in red, the 10-28 Fabs in yellow, and the 11-11 Fabs in dark green. The Fab heavy chains are shaded darker, and the light chain shaded lighter. The footprint of each respective antibody on the inner face of RBD is displayed next to each spike. FIG. 8B shows crystal structure of the 10-40 Fab bound to USA-WA1/2020 SARS-COV-2 RBD. FIG. 8C shows a footprint comparison of 10-28, 10-40, DH1047, COVA-1-16, and S2X259 epitope footprints on SARS-COV-2 RBD. The RBD is colored according to the sequence conservation of each residue across 52 sarbecoviruses. FIG. 8D shows a comparison of 10-40, COVA1-16, and 2-36 CDRH3s produced by superimposing RBDs from each complex. The CDRH3s of the three antibodies bind to RBD with a highly similar mode. FIG. 8E shows heavy chain sequence alignment of antibodies 10-40, 2-36, C022, and COVA1-16, with the CDRH3 aligned with the germline sequence of the IGHD3-22*01 gene. The paratope residues are underlined and residues that form hydrogen bonds with RBD are colored in red. FIG. 8F shows D gene usage in antibody repertoires among healthy donors, ranked by frequency.

FIGS. 9A-E show prophylactic protection against a mouse adapted strain of SARSCOV-2 by engineered monoclonal antibody 10-40. FIG. 9A shows that mutations in the MA10 virus do not overlap with the epitope of 10-40. The binding epitope of 10-40 is denoted in purple and the ACE2 binding epitope is denoted in blue. FIG. 9B shows neutralization of the mouse adapted strain of SARS-COV-2 (MA10) by antibody 10-40 in Vero E6 cells. FIG. 9C shows an experimental timeline of a protection study in SARS-COV-2 MA10 challenged mice. FIG. 9D shows body weight change in each group. Percent changes in body weight change of individual mice at day 3 shown (N=8 mice/group). Two-tailed t-test with Wilcoxon matched-pairing used to determine significance indicated by p values. FIG. 9E shows TCID50/gm of lung from 5-fold titration of lung homogenate of each mice.

FIGS. 10A-B show neutralization profiles of the convalescent sera from COVID-19 patients selected for antibody isolation (FIG. 10A) and a summary of ID₅₀ titers (ID₅₀ is the estimated number of virus particles required to produce infection in 50% of normal adult humans exposed) against each virus (FIG. 10B). Sera from two COVID-19 patients are tested for neutralization against VSV pseudotyped with the spike of SARS-COV-2 variants or SARS-COV spikes. Data are shown as mean±SD of three technical replicates.

FIGS. 11A-L show individual binding curves to Spike trimer for antibodies disclosed herein as measured by competition ELISA. Data are shown as mean+SD of two technical replicates. FIG. 11A shows individual binding curves to Spike trimer where the antibodies disclosed herein compete with biotinylated 10-40 antibody. FIG. 11B shows individual binding curves to Spike trimer where the antibodies disclosed herein compete with biotinylated 10-28 antibody. FIG. 11C shows individual binding curves to Spike trimer where the antibodies disclosed herein compete with biotinylated 11-11 antibody. FIG. 11D shows individual binding curves to Spike trimer where the antibodies disclosed herein compete with biotinylated 2-36 antibody. FIG. 11E shows individual binding curves to Spike trimer where the antibodies disclosed herein compete with biotinylated DH1047 antibody. FIG. 11F shows individual binding curves to Spike trimer where the antibodies disclosed herein compete with biotinylated S2X259 antibody. FIG. 11G shows individual binding curves to Spike trimer where the antibodies disclosed herein compete with biotinylated REGN10985 antibody. FIG. 11H shows individual binding curves to Spike trimer where the antibodies disclosed herein compete with biotinylated ADG-2 antibody. FIG. 11I shows individual binding curves to Spike trimer where the antibodies disclosed herein compete with biotinylated COVA1-16 antibody. FIG. 11J shows individual binding curves to Spike trimer where the antibodies disclosed herein compete with biotinylated CR3022 antibody. FIG. 11K shows individual binding curves to Spike trimer where the antibodies disclosed herein compete with biotinylated S2H97 antibody. FIG. 11L shows individual binding curves to Spike trimer where the antibodies disclosed herein compete with biotinylated S309 antibody.

FIGS. 12A-C show binding kinetics and affinities of engineered mAbs 10-28, 10-40, and 11-11, and other reported broad antibodies. Binding of antibodies to non-variant SARS-COV-2 (FIG. 12A) and SARS-COV (FIG. 12B) spike proteins were determined by surface plasmon resonance (SPR). The spike protein bound sensors were incubated with six different concentrations of antibodies. Kinetic data from one representative experiment were fit to a 1:1 binding model. FIG. 12C shows a summary of SPR kinetic and affinity measurements.

FIGS. 13A-C shows a genetic analysis of engineered mAbs 10-40, 10-28, and 11-11. FIG. 13A shows genetic annotation for three broadly neutralizing engineered mAbs described herein, 10-28, 10-40, and 11-11. FIG. 13B shows a sequence alignment of engineered mAbs 10-40 with its germline genes, and gene specific substitution profile (GSSP) for IGHV4-39 and IGLV6-57. The dots denote conserved residues. The Y52T substitution in the 10-40 heavy chain is colored in red to indicate a rare mutation. FIG. 13C shows the signature of the 10-40-like reproducible antibody class.

FIGS. 14A-L show the neutralization profiles of antibodies against SARS-COV-2 variants. Neutralization curves are shown of antibodies of the present disclosure against vesicular stomatitis virus (VSV) pseudotypes with SARS-COV-2 spike proteins. FIG. 14A shows neutralization curves of engineered mAb 10-40 against SARS-COV-2 variants. FIG. 14B shows neutralization curves of engineered mAb 10-28 against SARS-COV-2 variants. FIG. 14C shows neutralization curves of engineered mAb 11-11 against SARS-COV-2 variants. FIG. 14D shows neutralization curves of mAb 2-36 against SARS-COV-2 variants. FIG. 14E shows neutralization curves of antibody DH1047 against SARS-COV-2 variants. FIG. 14F shows neutralization curves of antibody S2X259 against SARS-COV-2 variants. FIG. 14G shows neutralization curves of antibody REGN10985 against SARS-COV-2 variants. FIG. 14H shows neutralization curves of antibody ADG-2 against SARS-COV-2 variants. FIG. 14I shows neutralization curves of antibody COVA1-16 against SARS-COV-2 variants. FIG. 14J shows neutralization curves of antibody CR3022 against SARS-COV-2 variants. FIG. 14K shows neutralization curves of antibody S2H97 against SARS-COV-2 variants. FIG. 14L shows neutralization curves of antibody S309 against SARS-COV-2 variants. Data are shown as mean±SD of three technical replicates.

FIGS. 15A-M show neutralization profiles of antibodies against authentic SARS-CoV-2 variant viruses. Neutralization curves of antibodies against authentic SARS-COV-2 strains in a cytopathic effect reduction assay. Data are shown as mean±SEM of three technical replicates. FIG. 15A shows neutralization profiles of engineered mAb 10-40 against authentic SARS-COV-2 variant viruses. FIG. 15B shows neutralization profiles of engineered mAb 10-28 against authentic SARS-COV-2 variant viruses. FIG. 15C shows neutralization profiles of engineered mAb 11-11 against authentic SARS-COV-2 variant viruses. FIG. 15D shows neutralization profiles of mAb 2-36 against authentic SARS-COV-2 variant viruses. FIG. 15E shows neutralization profiles of antibody DH1047 against authentic SARS-COV-2 variant viruses. FIG. 15F shows neutralization profiles of antibody S2X259 against authentic SARS-COV-2 variant viruses. FIG. 15G shows neutralization profiles of antibody REGN10985 against authentic SARS-COV-2 variant viruses. FIG. 15H shows neutralization profiles of antibody ADG-2 against authentic SARS-COV-2 variant viruses. FIG. 15I shows neutralization profiles of antibody CAVA1-16 against authentic SARS-COV-2 variant viruses. FIG. 15J shows neutralization profiles of antibody CR3022 against authentic SARS-CoV-2 variant viruses. FIG. 15K shows neutralization profiles of antibody S2H97 against authentic SARS-COV-2 variant viruses. FIG. 15L shows neutralization profiles of antibody S309 against authentic SARS-COV-2 variant viruses. FIG. 15M shows neutralization profiles of antibodies 10-40, 117/40, DH1047, REGN10985, ADG2, S2X259, and 2-36 against authentic SARS-COV.

FIGS. 16A-L show neutralization profiles of antibodies against sarbecoviruses. Neutralization curves of antibodies against VSV pseudotypes with sarbecovirus spike proteins. FIG. 16A shows neutralization profiles of engineered mAb 10-40 against sarbecoviruses. FIG. 16B shows neutralization profiles of engineered mAb 10-28 against sarbecoviruses. FIG. 16C shows neutralization profiles of engineered mAb 11-11 against sarbecoviruses. FIG. 16D shows neutralization profiles of antibody 2-36 against sarbecoviruses. FIG. 16E shows neutralization profiles of antibody DH1047 against sarbecoviruses. FIG. 16F shows neutralization profiles of antibody S2X259 against sarbecoviruses. FIG. 16G shows neutralization profiles of antibody REGN10985 against sarbecoviruses. FIG. 16H shows neutralization profiles of antibody ADG-2 against sarbecoviruses. FIG. 16I shows neutralization profiles of antibody COVA1-16 against sarbecoviruses. FIG. 16J shows neutralization profiles of antibody CR3022 against sarbecoviruses. FIG. 16K shows neutralization profiles of antibody S2H97 against sarbecoviruses. FIG. 16L shows neutralization profiles of antibody S309 against sarbecoviruses. Data are shown as mean±SD of three technical replicates.

FIGS. 17A-N show breadth of cross-reactive antibodies examined by RBD-based ELISA. FIG. 17A shows a phylogenetic tree of coronaviruses based on RBD protein sequences. Four phylogenetically distinct sarbecovirus groups are indicated in different colors. FIGS. 17B-N show binding of the selected antibodies disclosed herein to the panel of RBD proteins as tested by ELISA. FIG. 17B shows antibody binding to the BM-4831 RBD. FIG. 17C shows antibody binding to the BtKY72 RBD. FIG. 17D shows antibody binding to the SHC014 RBD. FIG. 17E shows antibody binding to the WIV1 RBD. FIG. 17F shows antibody binding to the SARS-COV RBD. FIG. 17G shows antibody binding to the GX-Pangolin RBD. FIG. 17H shows antibody binding to the RaTG13 RBD. FIG. 17I shows antibody binding to the SARS-COV-2 RBD. FIG. 17J shows antibody binding to the B.1.351 RBD. FIG. 17K shows antibody binding to the Rf1 RBD. FIG. 17L shows antibody binding to the RmYN02 RBD. FIG. 17M shows antibody binding to the Rs4081 RBD. FIG. 17N shows antibody binding to the Yunnan2011 RBD.

FIGS. 18A-B show binding of antibody 10-40 to coronavirus spike-expressing cells. FIG. 18A shows gating strategy for quantification of 10-40-positive cells from Expi293 cells transiently transfected to express coronavirus spike proteins. FIG. 18B shows the binding profiles of antibody 10-40 to spike trimers expressed on Expi293 cell surface.

FIG. 19 shows mean fluorescent intensity (MFI) of the antibodies disclosed herein bound to spike-expressing cells. The data shown are representative data from one of three independent experiments.

FIGS. 20A-E show Cryo-EM data processing for engineered mAb 10-40 in complex with SARS-COV-2 B.1.351 spike trimer. FIG. 20A shows representative micrograph, power spectrum, and contrast transfer function (CTF) fit. FIG. 20B shows representative 2D class averages showing spike particles. FIG. 20C shows global consensus refinement Fourier Shell Correlation (FSC) curve (top panel) and particle projection viewing angle distribution (bottom panel). FIG. 20D shows local resolution estimation mapped on surface density for global refinement. FIG. 20E shows crystal structure model for 10-40 Fab rigid body docked into cryo-EM map, viewed from the outside (left) and from the inside (right).

FIGS. 21A-E show Cryo-EM data processing for antibody 10-28 in complex with SARS-COV-2 B.1.351 spike trimer. FIG. 21A shows representative micrograph, power spectrum, and contrast transfer function (CTF) fit. FIG. 21B shows representative 2D class averages showing spike particles. FIG. 21C shows a global consensus refinement Fourier Shell Correlation (FSC) curve (top panel) and particle projection viewing angle distribution (bottom panel). FIG. 21D shows local resolution estimation mapped on surface density for global refinement. FIG. 21E shows a crystal structure model for 10-28 Fab rigid body docked into cryo-EM map, viewed from the outside (left) and from the inside (right).

FIGS. 22A-E show Cryo-EM data processing for antibody 11-11 in complex with SARS-COV-2 B.1.351 spike trimer. FIG. 22A shows representative micrograph, power spectrum, and contrast transfer function (CTF) fit. FIG. 22B shows representative 2D class averages showing spike particles. FIG. 22C shows global consensus refinement Fourier Shell Correlation (FSC) curve (top panel) and particle projection viewing angle distribution (bottom panel). FIG. 22D shows local resolution estimation mapped on surface density for global refinement. FIG. 22E shows homology model for 11-11 Fab rigid body docked into cryo-EM map, viewed from the outside (left) and from the inside (right).

FIGS. 23A-B show crystal structures. FIG. 23A shows crystal structure of antibody 10-28 complexed with non-variant SARS-COV-2 RBD. RBD is colored in green, heavy and light chains are in dark and light yellow. CL=constant light chain; CH=constant heavy chain; VL=variable light chain; VH=variable heavy chain. FIG. 23B shows expanded view of engineered mAb 10-28 interactions with RBD recognition by CDRL3 (upper panel), and recognition by CDRH3 (lower panel), colored as in (A).

FIGS. 24A-D show footprint comparison between engineered mAbs 10-40, 11-11, and 10-28, and published antibodies. FIG. 24A shows that antibodies 10-40, COVA1-16, and 2-36 recognize the inner side of the inner face of RBD with a similar angle of approach.

FIG. 24B shows that antibodies 11-11, S2X259, and DH1047 recognize the outer side of the inner face of RBD with a similar angle of approach. FIG. 24C shows that antibodies 10-28 and CR3022 bind to the lower middle side of the inner face of RBD. ADG-2 binds to the upper side of the inner face of RBD. Atomic coordinates are not available for ADG-2, thus, it is modeled by docking a homology model based on 6APC into the map EMD-23160, as described by (ADG-2 citation). FIG. 24D shows nanobodies that broadly neutralize also recognize the inner face of RBD from the outer side, similar to antibodies in FIG. 24B.

FIGS. 25A-C show that engineered mAb 10-40 recognizes a conserved region within the RBD. FIG. 25A shows a sequence alignment of sarbecoviruses within the epitope of 10-40. FIG. 25B shows sequence conservation of antibody 10-40 epitope in sarbecoviruses. FIG. 25C shows epitope residues for antibody 10-40 (red colored/underlined), compared with recently identified 5 related viruses. Blue represents human-ACE2 dependent viruses (BANAL-52, BANAL-103, and BANAL-236), yellow are non-ACE2 dependent viruses (BANAL-116 and BANAL-247).

FIG. 26 shows the signature of engineered mAb 10-40-like reproducible antibody class.

FIG. 27 shows Cryo-EM data collection and processing.

FIG. 28 shows X-ray diffraction data collection and refinement statistics.

FIGS. 29A-O show neutralization profiles of down-selected monoclonal antibodies (mAbs) disclosed herein on wild-type (WT) SARS-COV-2 virus and pseudoviruses of SARS-COV-2 variants of concern (VOC PVs). FIG. 29A shows neutralization profiles with mAbs REGN10933 and REGN10987. FIG. 29B shows neuralization profiles with mAb 2-7. FIG. 29C shows neutralization profiles with mAb 2-36. FIG. 29D shows neutralization profiles with mAbs LY-COV555 and CB6. FIG. 29E shows neutralization profiles with mAbs Brii-196 and Brii-198. FIG. 29F shows neutralization profiles with mAbs COV2-2196 and COV2-2130. FIG. 29G shows neutralization profiles with engineered mAb 10-16. FIG. 29H shows neutralization profiles with engineered mAb 10-28. FIG. 29I shows neutralization profiles with engineered mAb 10-40. FIG. 29J shows neutralization profiles with engineered mAb 11-11. FIG. 29K shows neutralization profiles with engineered mAb 12-6. FIG. 29L shows neutralization profiles with engineered mAb 12-16. FIG. 29M shows neutralization profiles with engineered mAb 12-19. FIG. 29N shows neutralization profiles with engineered mAb 12-27. FIG. 29O shows neutralization profiles with engineered mAb 13-2.

FIG. 30 shows the IC₅₀ values from neutralization of SARS-COV-2 virus and SARS-COV pseudoviruses with down-selected engineered monoclonal antibodies disclosed herein.

FIGS. 31A-E show characterization of the in vitro and in vivo potency and breadth of neutralizing engineered mAb 12-16 and 12-19. FIG. 31A shows neutralization titers (ID₅₀) of the convalescent serum selected for antibody isolation against VSV pseudotypes with the spike of SARS-COV-2 variants or SARS-COV spikes. Data are shown as mean±SD of three technical replicates. FIG. 31B shows neutralization titers (IC₅₀) of selected mAbs against VSV pseudotypes with SARS-COV-2 spikes and authentic viruses. Data are shown as mean±SD of three technical replicates. FIG. 31C shows that antibodies were tested for binding to the spike trimers of SARS-COV-2 D614G and B.1.351 by ELISA. Data are shown as mean±SD of two technical replicates. FIG. 31D shows antibody binding to the SARS-COV-2 D614G (yellow) and B.1.351 (blue) spike proteins expressed on the cell surface. The data shown are representative data from one of three independent experiments. FIG. 31E shows prophylactic efficacy of selected mAbs in hamsters infected with Omicron variant. Three groups of hamsters (n=6 per group) received 10 mg/kg of the indicated mAb treatment by intraperitoneal injection one day before intranasal inoculation with 105 PFU of Omicron variant. Viral load (log 10 (RNA copies/106 β-actin copies)) and viral titers (log 10 (PFU per g)) were measured in trachea, lung of hamsters challenged with Omicron virus at 4 d.p.i. Error bars indicate mean±SD. Each symbol represents an individual hamster with a line indicating the mean of each group. P values were determined by one-way ANOVA followed by Tukey's multiple comparisons test. **P<0.01; ***P<0.001; ****P<0.0001. Dotted lines indicate assay limits of detection.

FIGS. 32A-G show targeting by engineered monoclonal antibodies 12-16 and 12-19. FIG. 32A shows CryoEM structure of antibody 12-16, the panels show top view, side view and location of complementarity-determining region (CDRs). The orange and green surface represent the footprint of 12-16 on NTD and SD1, respectively. FIG. 32B shows CryoEM structures of antibodies 12-19, the panels showed top view, side view and location of CDRs. The orange and green surface represent the footprint of 12-19 on NTD and SD1, respectively. FIG. 32C shows antibodies 12-16 and 12-19 in complex with NTD and SD1. The red sphere represents the mutations in Omicron variant, the NTD antigenic supersite is outlined in cyan. FIG. 32D shows interface residues and interaction for antibody 12-16 with SD1 (left panel) and NTD (right panel). The hydrogen bonds are colored in yellow dashed lines. FIG. 32E shows interface residues and interaction for antibody 2-19 with SDI (left panel) and NTD (right panel). The hydrogen bonds are colored in yellow dashed lines. FIG. 32F shows the footprint and residue entropy for receptor-binding motif (RBM), NTD supersite, 12-16 and 12-19 epitope on the spike surface. FIG. 32G shows comparison of residue entropy between RBM, NTD supersite, 12-16 and 12-19 epitopes. Residues are represented as dots, and the geometric mean and 95% confidence interval (CI) of the 4 groups of residues are shown in blue, cyan, green and purple, respectively. The P values between different groups were calculated using the Kolmogorov-Smirnov test. The double asterisk symbol represents P<0.0001.

FIG. 33A-G show the mechanism of NTD-specific neutralizing engineered mAbs against SARS-COV-2. FIG. 33A shows 10 selected antibodies in complex with hACE2 and 1 receptor binding domain (RBD) up spike. FIG. 33B shows competition of selected antibodies with ACE2 to bind to SARS-COV-2 spike trimer expressed on Expi293 cell surface. FIG. 33C shows blockage of soluble ACE2-induced shedding of SI from the S trimer on SARS-CoV-2 virions by indicated antibodies. FIG. 33D shows that the intensities of the S1 and S2 glycoprotein bands in (C) were measured and the S1/S2 ratios are shown. FIG. 33E shows competition of selected antibodies with CB6 for binding to a SARS-COV-2 spike trimer expressed on Expi293 cell surface. FIG. 33F shows blockage of soluble CB6-induced shedding of SI from the S trimer on SARS-COV-2 virions by indicated antibodies. The data shown are representative data from one of three independent experiments. FIG. 33G shows that the intensities of the S1 and S2 glycoprotein bands in (F) were measured and the S1/S2 ratios are shown.

FIGS. 34A-E show structure-based neutralization mechanisms of engineered monoclonal antibodies 12-16, 12-19 and NTD. FIG. 34A shows superpose of RBD up and down protomers. FIG. 34B shows per residue Ca distance of RBD up and down protomer showed in A. The dashed lines showed the boundary of each domain. FIG. 34C shows structural explanation of 12-16 and 12-19 block RBD up. Right panel showed the details of clashes occurred in SDIRDB-up with 12-16 and 12-19, the green and cyan sphere represent the N331 glycan in RBD-down and RBD-up protomer, respectively. FIG. 34D shows neutralization activities of monoclonal antibodies 12-16, 12-19 and 4-18 compared with their F(ab′) 2 forms. FIG. 34E shows structure models for spike protein bound with neutralizing antibodies (12-16, 12-19 and NTD antigenic site antibodies).

FIGS. 35A-B show neutralization profiles of engineered mAbs 12-16 and 12-19 against SARS-COV-2 variants. FIG. 35A shows neutralization curves of antibodies against VSV pseudotypes with SARS-COV-2 spikes. FIG. 35B shows neutralization curves of antibodies against authentic D614G and B.1.1.529 viruses. Data are shown as mean±SD of three technical replicates.

FIGS. 36A-D show genetic analysis for engineered monoclonal antibodies 12-16 and 12-19. FIG. 36A shows germline gene assignment for antibodies 12-16 and 12-19. FIG. 36B shows gene specific substitution profile (GSSP) for antibody 12-16, the dots represent the conserved residues in antibody sequence compared with germline gene, the CDRs were highlighted by rectangles. FIG. 36C shows gene specific substitution profile (GSSP) for antibody 12-19, the dots represent the conserved residues in antibody sequence compared with germline gene, the CDRs were highlighted by rectangles. FIG. 36D shows CDRH3 VDJ junction analysis for 12-16 and 12-19. Germline nucleotide and amino acid residues are shown in black with the corresponding junctions colored in light blue. Somatic hypermutations are colored in red. Nucleotides deleted by exonuclease trimming are indicated with strikethrough. The blue nucleotides represent the N and P nucleotide additions at the junctions.

FIG. 37A-G show Cryo-EM data processing for engineered antibody 12-16 in complex with the SARS-COV-2 D614G spike trimer. FIG. 37A shows a representative micrograph showing spike particle distribution in ice. FIG. 37B shows a micrograph power spectrum (left) with contrast transfer function (CTF) fit (right). FIG. 37C shows selected 2D class averages showing initial spike particle set. FIG. 37D shows a global refinement Fourier Shell Correlation (FSC) curve showing overall resolution. FIG. 37E shows a map density shown as a mesh for antibody CDRH3 loop showing side chain fits. FIG. 37F shows local resolution mapped onto global refinement reconstruction. FIG. 37G shows local resolution for the antibody 12-16 interface, shown from the top and the bottom.

FIGS. 38A-G show Cryo-EM data processing for antibody 12-19 in complex with SARS-COV-2 D614G spike trimer. FIG. 38A shows a representative micrograph showing spike particle distribution in ice. FIG. 38B shows a micrograph power spectrum (left) with contrast transfer function (CTF) fit (right). FIG. 38C shows selected 2D class averages showing initial spike particle set. FIG. 38D shows a global refinement Fourier Shell Correlation (FSC) curve showing overall resolution. FIG. 38E shows a map density shown as mesh for antibody CDRH3 loop showing side chain fits. FIG. 38F shows local resolution mapped onto global refinement reconstruction. FIG. 38G shows local resolution for the antibody 12-19 interface, shown from the top and the bottom.

FIGS. 39A-B shows the location of mutations in the Omicron BA.2 and P.1 variants in complex with antibodies 12-16 and 12-19. FIG. 39A shows antibodies 12-16 and 12-19 in complex with NTD and SD1. The red spheres represent the mutations in the Omicron BA.2 variant, the NTD antigenic supersite is outlined in cyan. FIG. 39B shows antibodies 12-16 and 12-19 in complex with NTD and SD1. The red spheres represent the mutations in P.1 variant.

FIGS. 40A-C show structural evidence for antibody neutralization mechanisms. FIG. 40A shows superpose of RBD up and down protomers. The surface shows the RBD down protomer and the ribbon shows the RBD up protomer (PDB: 7krr). FIG. 40B shows a structural model showing that the Fc or Fab of NTD supersite antibody clash with dimer hACE2. The full IgG structure was made by superimposing the heavy chains of the crystal IgG structure (PDB: 1IHZH) with the supersite antibody (PDB: 7L2F). FIG. 40C shows a comparison of antibodies 12-16 and 12-19 with NTD antibody 5-7 and SDI antibody S3H3.

FIGS. 41A-C show neutralization of VSV pseudotyped with SARS-COV-2 D614G spike protein by the selected IgG and F(ab′) 2. FIG. 41A shows neutralization curves of the selected IgG and F (ab′) 2 against VSV pseudotypes with SARS-COV-2 D614G spike protein. Data are shown as mean±SD of three technical replicates. FIG. 41B shows mAbs 50% inhibitory concentration (IC₅₀) for SARS-COV-2 D614G spike protein. Data are shown as mean±SD of three technical replicates. FIG. 41C shows SDS-PAGE results for purity determination of purified IgG (left panel) and F (ab′) 2 (right panel).

FIGS. 42A-C show that soluble ACE2 or CB6 induced the shedding of S1 from VSV particles pseudotyped with the SARS-COV-2 D614G S glycoproteins. FIG. 42A shows soluble ACE2 incubation. FIG. 42B shows CB6 incubation. FIG. 42C shows the intensities of the S1 and S2 glycoprotein bands in (A) and (B) were measured and the S1/S2 ratios for each concentration of sACE2 or CB6 are shown.

FIGS. 43A-B show binding kinetics and affinities of selected antibodies. FIG. 43A shows that binding of antibodies to SARS-COV-2 D614G spike protein was determined by surface plasmon resonance (SPR). FIG. 43B shows summary of SPR kinetic and affinity measurements.

FIG. 44 shows CryoEM data collection and model refinement.

FIGS. 45A-B show sequence alignment monoclonal antibodies disclosed herein. FIG. 45A shows multiple sequence alignment of the light chain variable domains of antibodies disclosed herein. FIG. 45B shows multiple sequence alignment of the heavy chain variable domains of antibodies disclosed herein.

FIGS. 46A-B shows identification of the complementarity determining regions (CDRs) of monoclonal antibodies. FIG. 46A shows sequence alignment of CDRs in the light chain variable domains. FIG. 46B shows sequence alignment of CDRs in the heavy chain variable domains.

FIG. 47 shows the amino acid sequences of the CDRs in antibody heavy chains.

FIG. 48 shows the amino acid sequences of the CDRs in antibody light chains.

### DETAILED DESCRIPTION OF THE INVENTION

The antibody is a heteromultimeric glycoprotein comprising at least two heavy chains and two light chains of amino acid sequences. Apart from IgM antibodies, intact monoclonal antibodies are usually heterotetrameric glycoproteins composed of two identical light (L) chains and two identical heavy (H) chains. Typically, each light chain is linked to a heavy chain by disulfide bonding. Each heavy and light chain also has intrachain disulfide bridges. Each heavy chain has at one end of the chain a variable domain (VH) followed by a number of constant regions. Each light chain also has a variable domain (VL) at one end of the chain and a constant region at the other end. The light chains of antibodies from most vertebrate species can be assigned to one of two types, called kappa and lambda. This assignment is based on the amino acid sequence of the constant region. The variable domains of an antibody confer binding specificity to the antibody for a particular antigen such as a surface protein of a virus. Certain regions of the variable domains exhibit unique variability (hypervariability) of their amino acid sequences and are called the complementarity determining regions (CDRs) of the antibody. The more conserved part of the variable region is called the framework region (FR). The intact heavy and light chain variable domains of an antibody each contain four FRs joined by three CDRs. The CDRs in each chain are held close together by the FR region together with the CDRs from the other chain and contribute to the formation of the antigen binding site of the antibody.

In some embodiments, the subject matter disclosed herein relates to the isolation, characterization and sequencing of potent and broadly neutralizing monoclonal antibodies (mAbs) against SARS-COV-2 and its variants as well as related coronaviruses. In some embodiments, the mAbs are isolated from patient blood samples. In some embodiments, the mAbs are isolated from patient serum samples. In some embodiments, the patients are infected with a SARS-COV-2 (WA1) virus. In some embodiments, the patients are infected with SARS-COV-2 variant(s) harboring the E484K mutation. In some embodiments, the patents are infected with a SARS-COV-2 D614G variant virus. In some embodiments, the patents are infected with a SARS-COV-2 B.1.351 variant virus. In some embodiments, the patents are infected with a SARS-COV-2 B.1.526 variant virus. In some embodiments, the patents are infected with a SARS-COV-2 B.1.617.1 variant virus. In some embodiments, the patents are infected with a SARS-COV-2 B.1.1.1 variant virus. In some embodiments, the patents are infected with a SARS-COV-2 B.1.1.7 variant virus. In some embodiments, the patents are infected with a SARS-COV-2 P.1 variant virus. In some embodiments, the patents are infected with a SARS-COV-2 B.1.429 variant virus. In some embodiments, the patents are infected with a SARS-COV-2 B.1.617.2 variant virus. In some embodiments, the patents are infected with a SARS-COV-2 B.1.526-E484K variant virus. In some embodiments, the patents are infected with a SARS-COV-2 V23P12 variant virus. In some embodiments, the patents are infected with a SARS-COV virus. In some embodiments, the patients are infected with SARS-COV-2 through vaccination against COVID-19.

In some embodiments, the subject matter disclosed herein relates to the engineering of potent and broadly neutralizing monoclonal antibodies against SARS-COV-2 and its variants as well as related coronaviruses. In some embodiments, peripheral blood mononuclear cells (PBMCs) are isolated from COVID-19 patients. In some embodiments, the patients are infected with a SARS-COV-2 (WA1) virus. In some embodiments, the patients are infected with SARS-COV-2 variant(s) harboring the E484K mutation. In some embodiments, the patients are infected with a SARS-COV-2 D614G variant virus. In some embodiments, the patients are infected with a SARS-COV-2 B.1.351 variant virus. In some embodiments, the patients are infected with a SARS-COV-2 B.1.526 variant virus. In some embodiments, the patients are infected with a SARS-COV-2 B.1.617.1 variant virus. In some embodiments, the patients are infected with a SARS-COV-2 B.1.1.1 variant virus. In some embodiments, the patients are infected with a SARS-COV-2 B.1.1.7 variant virus. In some embodiments, the patients are infected with a SARS-COV-2 P.1 variant virus. In some embodiments, the patients are infected with a SARS-COV-2 B.1.429 variant virus. In some embodiments, the patients are infected with a SARS-COV-2 B.1.617.2 variant virus. In some embodiments, the patients are infected with a SARS-COV-2 B.1.526-E484K variant virus. In some embodiments, the patients are infected with a SARS-COV-2 V23P12 variant virus. In some embodiments, the patients are infected with a SARS-COV virus. In some embodiments, the patients are infected with SARS-COV-2 through vaccination against COVID-19. In some embodiments, the isolated PBMCs are sorted using FACS analysis. In some embodiments, the isolated PBMCs are sorted using any suitable method known in the art. In some embodiments, the PBMCs are sorted for the presence of a SARS-COV-2 spike(S) protein specific memory B cells. In some embodiments, paired antibody sequences are obtained from the S-trimer specific memory B cells. In some embodiments, the sequences are obtained using single-cell RNA-sequencing. In some embodiments, the antibody sequences are obtained using any suitable method known in the art. In some embodiments, mAbs are engineered in vitro from the obtained antibody sequences. In some embodiments, the engineered mAbs express the immunoglobulin variable regions of the mAbs isolated from patients' blood samples. In some embodiments, mAbs are engineered in vitro from the obtained antibody sequences after the sequences have been modified or optimized. In some embodiments, the engineered antibodies are generated using any suitable expression or vector system known in the art.

In some embodiments, the subject matter disclosed herein relates to the characterization of the engineered mAbs specific for SAR-COV-2, its variants, or related coronaviruses. In some embodiments, the binding profiles of the mAbs are characterized for binding to spike proteins of SAR-COV-2, its variants, or related coronaviruses. In some embodiments, the neutralization profiles of the mAbs are characterized for neutralization of SAR-COV-2, its variants, or related coronaviruses. Following characterization, the mAbs described herein show exceptional potency and breadth against the original SARS-COV-2 virus, multiple variants of concern (VOC) and variants of interest (VOI). In some embodiments, the antibodies described herein demonstrate significant potency and breadth against SARS-COV-1 and other SARS like coronaviruses.

In some embodiments, the subject matter described herein relates to methods of use of the engineered, neutralizing mAbs disclosed herein for treatment of patients infected with SARS-COV-2, its variants, or related coronaviruses. In some embodiments, the subject matter disclosed herein relates to the methods of use of the engineered mAbs described herein to treat future SARS-like coronavirus infections. In some embodiments, the engineered mAb described herein reduce viral load in coronavirus-infected patients. In some embodiments, the engineered antibodies described herein ameliorate disease severity in coronavirus-infected patients. In some embodiments, the engineered mAbs described herein improve the coronavirus-infected patient's clinical outcome. In some embodiments, the coronavirus is a SARS-COV-2 (WA1) virus. In some embodiments, the coronavirus is a SARS-COV-2 variant(s) harboring the E484K mutation. In some embodiments, the coronavirus is a SARS-CoV-2 D614G variant virus. In some embodiments, the coronavirus is a SARS-COV-2 B.1.351 variant virus. In some embodiments, the coronavirus is a SARS-COV-2 B.1.526 variant virus. In some embodiments, the coronavirus is a SARS-COV-2 B.1.617.1 variant virus. In some embodiments, the coronavirus is a SARS-COV-2 B.1.1.1 variant virus. In some embodiments, the coronavirus is a SARS-COV-2 B.1.1.7 variant virus. In some embodiments, the coronavirus is a SARS-COV-2 P.1 variant virus. In some embodiments, the coronavirus is a SARS-COV-2 B.1.429 variant virus. In some embodiments, the coronavirus is a SARS-CoV-2 B.1.617.2 variant virus. In some embodiments, the coronavirus is a SARS-COV-2 B.1.526-E484K variant virus. In some embodiments, the coronavirus is a SARS-COV-2 V23P12 variant virus. In some embodiments, the coronavirus is a SARS-COV virus. In some embodiments, the coronavirus is a SARS-like coronavirus.

In some embodiments, the subject matter disclosed herein relates to the methods of use of the engineered mAbs described herein as prophylaxis to prevent humans from becoming infected with a coronavirus. In some embodiments, the humans are at high risk of becoming infected with a coronavirus. In some embodiments, the humans are at low risk of becoming infected with a coronavirus. In some embodiments, the humans are members of the general population. In some embodiments, the humans are medical personal. In some embodiments, the humans are first responders. In some embodiments, the humans have previously been vaccinated against a coronavirus infection. In some embodiments, the humans have not been vaccinated for coronavirus infection. In some embodiments, the subject matter described herein relates to the methods of use of the engineered mAbs described herein as prophylaxis to prevent future outbreak(s) caused by SARS-COV-2, its variants or related SARS-like coronaviruses. In some embodiments, the coronavirus is a SARS-COV-2 (WA1) virus. In some embodiments, the coronavirus is a SARS-COV-2 variant(s) harboring the E484K mutation. In some embodiments, the coronavirus is a SARS-CoV-2 D614G variant virus. In some embodiments, the coronavirus is a SARS-COV-2 B.1.351 variant virus. In some embodiments, the coronavirus is a SARS-COV-2 B.1.526 variant virus. In some embodiments, the coronavirus is a SARS-COV-2 B.1.617.1 variant virus. In some embodiments, the coronavirus is a SARS-COV-2 B.1.1.1 variant virus. In some embodiments, the coronavirus is a SARS-COV-2 B.1.1.7 variant virus. In some embodiments, the coronavirus is a SARS-COV-2 P.1 variant virus. In some embodiments, the coronavirus is a SARS-COV-2 B.1.429 variant virus. In some embodiments, the coronavirus is a SARS-CoV-2 B.1.617.2 variant virus. In some embodiments, the coronavirus is a SARS-COV-2 B.1.526-E484K variant virus. In some embodiments, the coronavirus is a SARS-COV-2 V23P12 variant virus. In some embodiments, the coronavirus is a SARS-COV virus. In some embodiments, the coronavirus is a SARS-like coronavirus.

In some embodiments, the engineered mAbs described herein show great neutralization potency and breadth against SARS-COV-2, its variants, and other SARS-like coronaviruses. In some embodiments, the engineered mAbs described herein are pan-coronavirus therapeutics for the prevention and treatment for future coronavirus outbreaks. In some embodiment, the engineered mAbs described herein are administered to subjects as a single therapy course. In some embodiments, the engineered mAbs described herein are administered to subjects in combination with one or more antiviral therapies. In some embodiments, the engineered mAbs described herein are administered to subjects in a cocktail of two or more mAbs. In some embodiments, the cocktail includes one or more engineered antibody described here and one or more antibodies known in the art specific for coronaviruses. In some embodiments, the engineered mAbs described herein can be administered to a subject in combination therapy with any other suitable medicament known in the art. In some embodiments the subject is a human. In some embodiments, the subject is a human patient. In some embodiments, the subject is infected with a SARS-COV-2 virus, a SARS-COV-2 variant varus, or a SARS-related coronavirus. In some embodiments, the subject is suspected to be infected with a SARS-COV-2 virus, a SARS-COV-2 variant varus, or a SARS-related coronavirus. In some embodiments, the subject is at risk of being infected with a SARS-COV-2 virus, a SARS-COV-2 variant varus, or a SARS-related coronavirus. In some embodiments, the subject is at high risk of being infected with a SARS-COV-2 virus, a SARS-COV-2 variant varus, or a SARS-related coronavirus.

In some embodiments, the subject matter described herein relates to the identification of antibodies from convalescent serum as described below:

1. In some embodiments, serum samples from patients (vaccinated or unvaccinated) infected with a SARS-COV-2 virus, a SARS-COV-2 variant, or a related coronavirus are isolated and evaluated for the ability of potential antibodies contained within these serum samples to neutralize SARS-COV-2, various variants of concern, and/or related coronaviruses.

2. In some embodiments, peripheral blood mononuclear cells (PMBCs) are isolated from those patients that exhibited the strongest activity in number 1 above in order to isolate S trimer-specific memory B cells. In some embodiments, the S trimer is the SARS-CoV-2 B.1.351 S trimer. In some embodiments, the antibody sequences are recovered from these single B cells by high throughput sequencing.

3. In some embodiments, genes from single B cells encoding for these mAb sequences are synthesized and cloned into one or more expression vectors. In some embodiments, these engineered mAbs are expressed in vitro and purified for subsequent analyses and characterization.

4. In some embodiments, the engineered purified mAbs are tested for their ability to neutralize SARS-COV-2, its variants (by both pseudotyped virus assay and live virus assay), and related coronaviruses.

5. In some embodiments, the engineered antibodies described herein have emerged as top hits of the analyses with good antiviral activity against SARS-COV-2, multiple variants of concern (VOC) and variants of interest (VOI), and related coronaviruses.

In some embodiments, the high potency of the engineered mAbs described herein allows for smaller amounts to be administered to subjects to achieve the desired clinical effects. This could be through lower dosages and therefore less potential side effects, less frequent dosing regimens, or lower antibody production costs. It can also mean faster recovery from disease and greater efficacy of the engineered mAbs overall.

In some embodiments, the subject matter disclosed herein relates to the mAbs described herein targeting multiple epitopes. In some embodiments, the combination cocktails of two or more of the engineered mAb described herein provide even greater efficacy and lower the possibility of viral resistance. In some embodiments, the subject matter disclosed herein relates to the use of the engineered mAb described herein for diagnosis of COVID-19 exposure and utilization in laboratory research and development activities.

In certain aspects, the subject matter described herein provides an engineered monoclonal antibody, or a functional fragment thereof, selectively binding and neutralizing a at least a portion of a virus, wherein the antibody, or functional fragment thereof, comprises an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1, at least 60%, 62%, 65%, 67%, 70%, 72%, 75%, 77%, 80%, 82%, 85%, 87%, 90%, 92%, 95%, 97%, 98%, 99% identical to SEQ ID NO: 2, at least 60%, 62%, 65%, 67%, 70%, 72%, 75%, 77%, 80%, 82%, 85%, 87%, 90%, 92%, 95%, 97%, 98%, 99% identical to SEQ ID NO: 3, at least 60%, 62%, 65%, 67%, 70%, 72%, 75%, 77%, 80%, 82%, 85%, 87%, 90%, 92%, 95%, 97%, 98%, 99% identical to SEQ ID NO: 4, at least 60%, 62%, 65%, 67%, 70%, 72%, 75%, 77%, 80%, 82%, 85%, 87%, 90%, 92%, 95%, 97%, 98%, 99% identical to SEQ ID NO: 5, at least 60%, 62%, 65%, 67%, 70%, 72%, 75%, 77%, 80%, 82%, 85%, 87%, 90%, 92%, 95%, 97%, 98%, 99% identical to SEQ ID NO: 6, at least 60%, 62%, 65%, 67%, 70%, 72%, 75%, 77%, 80%, 82%, 85%, 87%, 90%, 92%, 95%, 97%, 98%, 99% identical to SEQ ID NO: 7, at least 60%, 62%, 65%, 67%, 70%, 72%, 75%, 77%, 80%, 82%, 85%, 87%, 90%, 92%, 95%, 97%, 98%, 99% identical to SEQ ID NO: 8, at least 60%, 62%, 65%, 67%, 70%, 72%, 75%, 77%, 80%, 82%, 85%, 87%, 90%, 92%, 95%, 97%, 98%, 99% identical to SEQ ID NO: 9, at least 60%, 62%, 65%, 67%, 70%, 72%, 75%, 77%, 80%, 82%, 85%, 87%, 90%, 92%, 95%, 97%, 98%, 99% identical to SEQ ID NO: 10, at least 60%, 62%, 65%, 67%, 70%, 72%, 75%, 77%, 80%, 82%, 85%, 87%, 90%, 92%, 95%, 97%, 98%, 99% identical to SEQ ID NO: 11, at least 60%, 62%, 65%, 67%, 70%, 72%, 75%, 77%, 80%, 82%, 85%, 87%, 90%, 92%, 95%, 97%, 98%, 99% identical to SEQ ID NO: 12, at least 60%, 62%, 65%, 67%, 70%, 72%, 75%, 77%, 80%, 82%, 85%, 87%, 90%, 92%, 95%, 97%, 98%, 99% identical to SEQ ID NO: 13, or at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence identical to SEQ ID NO: 1 and a light chain comprising an amino acid sequence identical to SEQ ID NO: 2. In some embodiments, the antibody or functional fragment thereof comprises a heavy chain comprising an amino acid sequence identical to SEQ ID NO: 3 and a light chain comprising an amino acid sequence identical to SEQ ID NO: 4. In some embodiments, the antibody or functional fragment thereof comprises a heavy chain comprising an amino acid sequence identical to SEQ ID NO: 5 and a light chain comprising an amino acid sequence identical SEQ ID NO: 6. In some embodiments, the antibody or functional fragment thereof comprises a heavy chain comprising an amino acid sequence identical to SEQ ID NO: 7 and a light chain comprising an amino acid sequence identical to SEQ ID NO: 8. In some embodiments, the antibody or functional fragment thereof comprises a heavy chain comprising an amino acid sequence identical to SEQ ID NO: 9 and a light chain comprising an amino acid sequence identical to SEQ ID NO: 10. In some embodiments, the antibody or functional fragment thereof comprises a heavy chain comprising an amino acid sequence identical to SEQ ID NO: 11 and a light chain comprising an amino acid sequence identical to SEQ ID NO: 12. In some embodiments, the antibody or functional fragment thereof comprises a heavy chain comprising an amino acid sequence identical to SEQ ID NO: 13 and a light chain comprising an amino acid sequence identical to SEQ ID NO: 14.

In some embodiments, the antibody or functional fragment thereof comprises a heavy chain comprising an amino acid sequence comprising the complementarity-determining regions (CDRs) of SEQ ID NO: 1 and a light chain comprising an amino acid sequence comprising the complementarity-determining regions (CDRs) of SEQ ID NO: 2. In some embodiments, the antibody or functional fragment thereof comprises a heavy chain comprising an amino acid sequence comprising the complementarity-determining regions (CDRs) of SEQ ID NO: 3 and a light chain comprising an amino acid sequence comprising the complementarity-determining regions (CDRs) of SEQ ID NO: 4. In some embodiments, the antibody or functional fragment thereof comprises a heavy chain comprising an amino acid sequence comprising the complementarity-determining regions (CDRs) of SEQ ID NO: 5 and a light chain comprising an amino acid sequence comprising the complementarity-determining regions (CDRs) of SEQ ID NO: 6. In some embodiments, the antibody or functional fragment thereof comprises a heavy chain comprising an amino acid sequence comprising the complementarity-determining regions (CDRs) of SEQ ID NO: 7 and a light chain comprising an amino acid sequence comprising the complementarity-determining regions (CDRs) of SEQ ID NO: 8. In some embodiments, the antibody or functional fragment thereof comprises a heavy chain comprising an amino acid sequence comprising the complementarity-determining regions (CDRs) of SEQ ID NO: 9 and a light chain comprising an amino acid sequence comprising the complementarity-determining regions (CDRs) of SEQ ID NO: 10. In some embodiments, the antibody or functional fragment thereof comprises a heavy chain comprising an amino acid sequence comprising the complementarity-determining regions (CDRs) of SEQ ID NO: 11 and a light chain comprising an amino acid sequence comprising the complementarity-determining regions (CDRs) of SEQ ID NO: 12. In some embodiments, the antibody or functional fragment thereof comprises a heavy chain comprising an amino acid sequence comprising the complementarity-determining regions (CDRs) of SEQ ID NO: 13 and a light chain comprising an amino acid sequence comprising the complementarity-determining regions (CDRs) of SEQ ID NO: 14.

In some embodiments, the virus is a coronavirus. In some embodiments, the coronavirus is selected from the coronaviruses consisting of SARS-COV-2, SARS-COV, MERS-COV, 229E, NL63, OC43, BM-4831, BtKY72, Rfl, RmYN02, Rs4081, Yunnan2011, ZC45, As6526, Rs4237, Rs4081, and HKU1. In some embodiments, the coronavirus is a SARS-COV-2 virus. In some embodiments, the SARS-COV-2 virus is a wild-type strain. In some embodiments, the SARS-COV-2 virus is a SARS-COV-2 D614G strain. In some embodiments, the SARS-COV-2 virus is a WA1 strain. In some embodiments, the SARS-CoV-2 virus is a B.1.1.7 strain. In some embodiments, the SARS-COV-2 virus is a B.1.351 strain. In some embodiments, the SARS-COV-2 virus is a P.1 strain. In some embodiments, the SARS-COV-2 virus is a B.1.526 strain. In some embodiments, the SARS-COV-2 virus is a B.1.429 strain. In some embodiments, the SARS-COV-2 virus is a B.1.617.1 strain. In some embodiments, the SARS-COV-2 virus is a B.1.1.1 strain. In some embodiments, the SARS-CoV-2 virus is a B.1.617.2 strain. In some embodiments, the SARS-COV-2 virus is a B.1.526-E484K strain. In some embodiments, the SARS-COV-2 virus is a V236P12 strain. In some embodiments, the coronavirus is a SARS-COV virus. In some embodiments, the coronavirus is a MERS-COV virus. In some embodiments, the coronavirus is a NL63 virus. In some embodiments, the coronavirus is a GD-Pangolin virus. In some embodiments, the coronavirus is a GX-Pangolin virus. In some embodiments, the coronavirus is a RaTG13 virus. In some embodiments, the coronavirus is a WIV1 virus. In some embodiments, the coronavirus is a SHC014 virus. In some embodiments, the coronavirus is a LYRa11 virus. In some embodiments, the coronavirus is a Rs7327 virus. In some embodiments, the coronavirus is a Rs4231 virus. In some embodiments, the coronavirus is a Rs4084 virus.

In certain aspects, the subject matter described herein provides a method for treating a subject having a viral infection or preventing a subject from developing a viral infection, the method comprising administering to the subject a therapeutically effective amount of a composition comprising an engineered monoclonal antibody or a functional fragment thereof.

In some embodiments, the antibody comprises an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1, at least 60%, 62%, 65%, 67%, 70%, 72%, 75%, 77%, 80%, 82%, 85%, 87%, 90%, 92%, 95%, 97%, 98%, 99% identical to SEQ ID NO: 2, at least 60%, 62%, 65%, 67%, 70%, 72%, 75%, 77%, 80%, 82%, 85%, 87%, 90%, 92%, 95%, 97%, 98%, 99% identical to SEQ ID NO: 3, at least 60%, 62%, 65%, 67%, 70%, 72%, 75%, 77%, 80%, 82%, 85%, 87%, 90%, 92%, 95%, 97%, 98%, 99% identical to SEQ ID NO: 4, at least 60%, 62%, 65%, 67%, 70%, 72%, 75%, 77%, 80%, 82%, 85%, 87%, 90%, 92%, 95%, 97%, 98%, 99% identical to SEQ ID NO: 5, at least 60%, 62%, 65%, 67%, 70%, 72%, 75%, 77%, 80%, 82%, 85%, 87%, 90%, 92%, 95%, 97%, 98%, 99% identical to SEQ ID NO: 6, at least 60%, 62%, 65%, 67%, 70%, 72%, 75%, 77%, 80%, 82%, 85%, 87%, 90%, 92%, 95%, 97%, 98%, 99% identical to SEQ ID NO: 7, at least 60%, 62%, 65%, 67%, 70%, 72%, 75%, 77%, 80%, 82%, 85%, 87%, 90%, 92%, 95%, 97%, 98%, 99% identical to SEQ ID NO: 8, at least 60%, 62%, 65%, 67%, 70%, 72%, 75%, 77%, 80%, 82%, 85%, 87%, 90%, 92%, 95%, 97%, 98%, 99% identical to SEQ ID NO: 9, at least 60%, 62%, 65%, 67%, 70%, 72%, 75%, 77%, 80%, 82%, 85%, 87%, 90%, 92%, 95%, 97%, 98%, 99% identical to SEQ ID NO: 10, at least 60%, 62%, 65%, 67%, 70%, 72%, 75%, 77%, 80%, 82%, 85%, 87%, 90%, 92%, 95%, 97%, 98%, 99% identical to SEQ ID NO: 11, at least 60%, 62%, 65%, 67%, 70%, 72%, 75%, 77%, 80%, 82%, 85%, 87%, 90%, 92%, 95%, 97%, 98%, 99% identical to SEQ ID NO: 12, at least 60%, 62%, 65%, 67%, 70%, 72%, 75%, 77%, 80%, 82%, 85%, 87%, 90%, 92%, 95%, 97%, 98%, 99% identical to SEQ ID NO: 13, or at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence identical to SEQ ID NO: 1 and a light chain comprising an amino acid sequence identical to SEQ ID NO: 2. In some embodiments, the antibody or functional fragment thereof comprises a heavy chain comprising an amino acid sequence identical to SEQ ID NO: 3 and a light chain comprising an amino acid sequence identical to SEQ ID NO: 4. In some embodiments, the antibody or functional fragment thereof comprises a heavy chain comprising an amino acid sequence identical to SEQ ID NO: 5 and a light chain comprising an amino acid sequence identical to SEQ ID NO: 6. In some embodiments, the antibody or functional fragment thereof comprises a heavy chain comprising an amino acid sequence identical to SEQ ID NO: 7 and a light chain comprising an amino acid sequence identical to SEQ ID NO: 8. In some embodiments, the antibody or functional fragment thereof comprises a heavy chain comprising an amino acid sequence identical to SEQ ID NO: 9 and a light chain comprising an amino acid sequence identical to SEQ ID NO: 10. In some embodiments, the antibody or functional fragment thereof comprises a heavy chain comprising an amino acid sequence identical to SEQ ID NO: 11 and a light chain comprising an amino acid sequence identical to SEQ ID NO: 12. In some embodiments, the antibody or functional fragment thereof comprises a heavy chain comprising an amino acid sequence identical to SEQ ID NO: 13 and a light chain comprising an amino acid sequence identical to SEQ ID NO: 14.

In some embodiments, the antibody or functional fragment thereof comprises a heavy chain comprising an amino acid sequence comprising the complementarity-determining regions (CDRs) of SEQ ID NO: 1 and a light chain comprising an amino acid sequence comprising the complementarity-determining regions (CDRs) of SEQ ID NO: 2. In some embodiments, the antibody or functional fragment thereof comprises a heavy chain comprising an amino acid sequence comprising the complementarity-determining regions (CDRs) of SEQ ID NO: 3 and a light chain comprising an amino acid sequence comprising the complementarity-determining regions (CDRs) of SEQ ID NO: 4. In some embodiments, the antibody or functional fragment thereof comprises a heavy chain comprising an amino acid sequence comprising the complementarity-determining regions (CDRs) of SEQ ID NO:

5 and a light chain comprising an amino acid sequence comprising the complementarity-determining regions (CDRs) of SEQ ID NO: 6. In some embodiments, the antibody or functional fragment thereof comprises a heavy chain comprising an amino acid sequence comprising the complementarity-determining regions (CDRs) of SEQ ID NO: 7 and a light chain comprising an amino acid sequence comprising the complementarity-determining regions (CDRs) of SEQ ID NO: 8. In some embodiments, the antibody or functional fragment thereof comprises a heavy chain comprising an amino acid sequence comprising the complementarity-determining regions (CDRs) of SEQ ID NO: 9 and a light chain comprising an amino acid sequence comprising the complementarity-determining regions (CDRs) of SEQ ID NO: 10. In some embodiments, the antibody or functional fragment thereof comprises a heavy chain comprising an amino acid sequence comprising the complementarity-determining regions (CDRs) of SEQ ID NO: 11 and a light chain comprising an amino acid sequence comprising the complementarity-determining regions (CDRs) of SEQ ID NO: 12. In some embodiments, the antibody or functional fragment thereof comprises a heavy chain comprising an amino acid sequence comprising the complementarity-determining regions (CDRs) of SEQ ID NO: 13 and a light chain comprising an amino acid sequence comprising the complementarity-determining regions (CDRs) of SEQ ID NO: 14.

In some embodiments, the virus is a coronavirus. In some embodiments, the coronavirus is selected from the strains consisting of SARS-COV-2, SARS-COV, MERS-CoV, 229E, NL63, OC43, BM-4831, BtKY72, Rf1, RmYN02, Rs4081, Yunnan2011, ZC45, As6526, Rs4237, Rs4081, and HKU1. In some embodiments, the coronavirus is a SARS-CoV-2 virus. In some embodiments, the SARS-COV-2 virus is a wild-type strain. In some embodiments, the SARS-COV-2 virus is a SARS-COV-2 D614G strain. In some embodiments, the SARS-COV-2 virus is a WA1 strain. In some embodiments, the SARS-CoV-2 virus is a B.1.1.7 strain. In some embodiments, the SARS-COV-2 virus is a B.1.351 strain. In some embodiments, the SARS-COV-2 virus is a P.1 strain. In some embodiments, the SARS-COV-2 virus is a B.1.526 strain. In some embodiments, the SARS-COV-2 virus is a B.1.429 strain. In some embodiments, the SARS-COV-2 virus is a B.1.617.1 strain. In some embodiments, the SARS-COV-2 virus is a B.1.1.1 strain. In some embodiments, the SARS-CoV-2 virus is a B.1.617.2 strain. In some embodiments, the SARS-COV-2 virus is a B.1.526-E484K strain. In some embodiments, the SARS-COV-2 virus is a V236P12 strain. In some embodiments, the coronavirus is a SARS-COV virus. In some embodiments, the coronavirus is a MERS-COV virus. In some embodiments, the coronavirus is a NL63 virus. In some embodiments, the coronavirus is a GD-Pangolin virus. In some embodiments, the coronavirus is a GX-Pangolin virus. In some embodiments, the coronavirus is a RaTG13 virus. In some embodiments, the coronavirus is a WIV1 virus. In some embodiments, the coronavirus is a SHC014 virus. In some embodiments, the coronavirus is a LYRa11 virus. In some embodiments, the coronavirus is a Rs7327 virus. In some embodiments, the coronavirus is a Rs4231 virus. In some embodiments, the coronavirus is a Rs4084 virus.

In some embodiments, the composition comprises a cocktail of two or more engineered monoclonal antibodies. In some embodiments, the composition further comprises an antiviral agent.

In certain aspects, the subject matter described herein provides a method of producing engineered monoclonal antibodies, the method comprising: a) isolating a biological sample from a subject infected with a virus; b) determining whether the biological sample has neutralizing activity against the virus; c) isolating peripheral blood mononuclear cells (PMBCs) from the subjects whose biological sample has neutralizing activity in step b); d) isolating coronavirus S protein trimer-specific memory single B cells from the PMBCs in c); e) recovering one of more monoclonal antibody sequences selective for a coronavirus S protein trimer from the B cells; f) determining the sequence encoding the one or more monoclonal antibodies recovered from the single B cells; g) cloning the sequence encoding the one or more monoclonal antibodies into one or more expression vectors; h) expressing one or more engineered monoclonal antibody in vitro; and i) purifying the one or more engineered monoclonal antibody.

In some embodiments, the biological sample is plasma. In some embodiments, the subject is not vaccinated against the virus. In some embodiments, the subject is vaccinated against the virus. In some embodiments, the determining in f) comprises high throughput sequencing.

In some embodiments, the virus is a coronavirus. In some embodiments, the coronavirus is selected from the strains consisting of SARS-COV-2, SARS-COV, MERS-CoV, 229E, NL63, OC43, BM-4831, BtKY72, Rf1, RmYN02, Rs4081, Yunnan2011, ZC45, As6526, Rs4237, Rs4081, and HKU1. In some embodiments, the coronavirus is a SARS-CoV-2 virus. In some embodiments, the SARS-COV-2 virus is a wild-type strain. In some embodiments, the SARS-COV-2 virus is a SARS-COV-2 D614G strain. In some embodiments, the SARS-COV-2 virus is a WA1 strain. In some embodiments, the SARS-CoV-2 virus is a B.1.1.7 strain. In some embodiments, the SARS-COV-2 virus is a B.1.351 strain. In some embodiments, the SARS-COV-2 virus is a P.1 strain. In some embodiments, the SARS-COV-2 virus is a B.1.526 strain. In some embodiments, the SARS-COV-2 virus is a B.1.429 strain. In some embodiments, the SARS-COV-2 virus is a B.1.617.1 strain. In some embodiments, the SARS-COV-2 virus is a B.1.1.1 strain. In some embodiments, the SARS-CoV-2 virus is a B.1.617.2 strain. In some embodiments, the SARS-COV-2 virus is a B.1.526-E484K strain. In some embodiments, the SARS-COV-2 virus is a V236P12 strain. In some embodiments, the coronavirus is a SARS-COV virus. In some embodiments, the coronavirus is a MERS-COV virus. In some embodiments, the coronavirus is a NL63 virus. In some embodiments, the coronavirus is a GD-Pangolin virus. In some embodiments, the coronavirus is a GX-Pangolin virus. In some embodiments, the coronavirus is a RaTG13 virus. In some embodiments, the coronavirus is a WIV1 virus. In some embodiments, the coronavirus is a SHC014 virus. In some embodiments, the coronavirus is a LYRa11 virus. In some embodiments, the coronavirus is a Rs7327 virus. In some embodiments, the coronavirus is a Rs4231 virus. In some embodiments, the coronavirus is a Rs4084 virus.

In certain aspects, the subject matter described herein provides an engineered monoclonal antibody, or a functional fragment thereof, wherein the antibody or fragment thereof selectively binds at least a portion of a virus.

In some embodiments, the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 17. In some embodiments, the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence identical to SEQ ID NO: 15 and a light chain comprising an amino acid sequence identical to SEQ ID NO: 16. In some embodiments, the antibody or functional fragment thereof comprises a heavy chain comprising an amino acid sequence identical to SEQ ID NO: 17 and a light chain comprising an amino acid sequence identical to SEQ ID NO: 18. In some embodiments, the antibody or functional fragment thereof comprises a heavy chain comprising an amino acid sequence comprising the complementarity-determining regions (CDRs) of SEQ ID NO: 15 and a light chain comprising an amino acid sequence comprising the complementarity-determining regions (CDRs) of SEQ ID NO: 16. In some embodiments, the antibody or functional fragment thereof comprises a heavy chain comprising an amino acid sequence comprising the complementarity-determining regions (CDRs) of SEQ ID NO: 17 and a light chain comprising an amino acid sequence comprising the complementarity-determining regions (CDRs) of SEQ ID NO: 18.

In some embodiments, the virus is a coronavirus. In some embodiments, the coronavirus is a coronavirus variant. In some embodiments, the coronavirus is a coronavirus-related virus. In some embodiments, the coronavirus is a wildtype SARS-COV-2 (D614G) virus. In some embodiments, the coronavirus is a SARS-COV-2 B.1.1.7 variant virus. In some embodiments, the coronavirus is a SARS-COV-2 B.1.351 variant virus. In some embodiments, the coronavirus is a SARS-COV-2 P.1 variant virus. In some embodiments, the coronavirus is a SARS-COV-2 B.1.526 variant virus. In some embodiments, the coronavirus is a SARS-COV-2 B.1.429 variant virus. In some embodiments, the coronavirus is a SARS-CoV-2 B.1.617.1v1 variant virus. In some embodiments, the coronavirus is a SARS-COV-2 B.1.617.2 variant virus. In some embodiments, the coronavirus is a SARS-COV-2 B.1.1.1 variant virus. In some embodiments, the coronavirus is a SARS-COV-2 C.37 variant virus. In some embodiments, the coronavirus is a SARS-COV-2 Omicron (B.1.1.529) variant virus. In some embodiments, the coronavirus is a SARS-COV-2 Omicron BA.1 (B.1.1.529.1) variant virus. In some embodiments, the coronavirus is a SARS-COV-2 Omicron BA.1.1 (B.1.1.529.1.1) variant virus. In some embodiments, the coronavirus is a SARS-COV-2

Omicron BA.2 (B.1.1.529.2) variant virus. In some embodiments, the coronavirus is a SARS-CoV-2 B.1.621 variant virus. In some embodiments, the coronavirus is a SARS-COV-2 WA1 virus. In some embodiments, the coronavirus is any virus of Table 1. In some embodiments, the coronavirus is a SARS-COV virus.

In some embodiments, the at least a portion of a virus comprises a coronavirus spike protein. In some embodiments, the at least a portion of a virus comprises a juncture between a subdomain 1 (SD1) and a N-Terminal Domain (NTD) of a coronavirus spike protein. In some embodiments, the at least a portion of a virus comprises a subdomain 1 (SD1) of a coronavirus spike protein. In some embodiments, the at least a portion of a virus comprises a N-Terminal Domain (NTD) of a coronavirus spike protein.

In certain aspects, the subject matter described herein provides a method for treating a subject having a viral infection or preventing a subject from developing a viral infection, the method comprising administering to the subject a therapeutically effective amount of a composition comprising an engineered monoclonal antibody or a functional fragment thereof, wherein the antibody or fragment thereof selectively binds at least a portion of the virus.

In some embodiments, the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 15. In some embodiments, the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 17. In some embodiments, the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16. In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence identical to SEQ ID NO: 15 and a light chain comprising an amino acid sequence identical to SEQ ID NO: 16. In some embodiments, the antibody or functional fragment thereof comprises a heavy chain comprising an amino acid sequence identical to SEQ ID NO: 17 and a light chain comprising an amino acid sequence identical to SEQ ID NO: 18.

In some embodiments, the antibody or functional fragment thereof comprises a heavy chain comprising an amino acid sequence comprising the complementarity-determining regions (CDRs) of SEQ ID NO: 15 and a light chain comprising an amino acid sequence comprising the complementarity-determining regions (CDRs) of SEQ ID NO: 16. In some embodiments, the antibody or functional fragment thereof comprises a heavy chain comprising an amino acid sequence comprising the complementarity-determining regions (CDRs) of SEQ ID NO: 17 and a light chain comprising an amino acid sequence comprising the complementarity-determining regions (CDRs) of SEQ ID NO: 18.

In some embodiments, the virus is a coronavirus. In some embodiments, the coronavirus is a coronavirus variant. In some embodiments, the coronavirus is a coronavirus-related virus. In some embodiments, the coronavirus is a wildtype SARS-COV-2 (D614G) virus. In some embodiments, the coronavirus is a SARS-COV-2 B.1.1.7 variant virus. In some embodiments, the coronavirus is a SARS-COV-2 B.1.351 variant virus. In some embodiments, the coronavirus is a SARS-COV-2 P.1 variant virus. In some embodiments, the coronavirus is a SARS-COV-2 B.1.526 variant virus. In some embodiments, the coronavirus is a SARS-COV-2 B.1.429 variant virus. In some embodiments, the coronavirus is a SARS-CoV-2 B.1.617.1v1 variant virus. In some embodiments, the coronavirus is a SARS-COV-2 B.1.617.2 variant virus. In some embodiments, the coronavirus is a SARS-COV-2 B.1.1.1 variant virus. In some embodiments, the coronavirus is a SARS-COV-2 D614G virus. In some embodiments, the coronavirus is a SARS-COV-2 C.37 variant virus. In some embodiments, the coronavirus is a SARS-COV-2 Omicron (B.1.1.529) variant virus. In some embodiments, the coronavirus is a SARS-COV-2 Omicron BA.1 (B.1.1.529.1) variant virus. In some embodiments, the coronavirus is a SARS-COV-2 Omicron BA.1.1 (B.1.1.529.1.1) variant virus. In some embodiments, the coronavirus is a SARS-COV-2 Omicron BA.2 (B.1.1.529.2) variant virus. In some embodiments, the coronavirus is a SARS-COV-2 B.1.621 variant virus. In some embodiments, the coronavirus is a SARS-COV-2 WA1 virus. In some embodiments, the coronavirus is any virus of Table 1. In some embodiments, the coronavirus is a SARS-CoV virus.

In some embodiments, the at least a portion of a virus comprises a coronavirus spike protein. In some embodiments, the at least a portion of a virus comprises a juncture between a subdomain 1 (SD1) and a N-Terminal Domain (NTD) of a coronavirus spike protein. In some embodiments, the at least a portion of a virus comprises a subdomain 1 (SD1) of a coronavirus spike protein. In some embodiments, the at least a portion of a virus comprises a N-Terminal Domain (NTD) of a coronavirus spike protein.

In certain aspects, the subject matter described herein provides a pharmaceutical composition comprising an engineered monoclonal antibody or a functional fragment thereof, wherein the antibody or fragment thereof selectively binds at least a portion of a virus.

In some embodiments, the antibody or fragment thereof comprises an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 1, at least 60%, 62%, 65%, 67%, 70%, 72%, 75%, 77%, 80%, 82%, 85%, 87%, 90%, 92%, 95%, 97%, 98%, 99% identical to SEQ ID NO: 2, at least 60%, 62%, 65%, 67%, 70%, 72%, 75%, 77%, 80%, 82%, 85%, 87%, 90%, 92%, 95%, 97%, 98%, 99% identical to SEQ ID NO: 3, at least 60%, 62%, 65%, 67%, 70%, 72%, 75%, 77%, 80%, 82%, 85%, 87%, 90%, 92%, 95%, 97%, 98%, 99% identical to SEQ ID NO: 4, at least 60%, 62%, 65%, 67%, 70%, 72%, 75%, 77%, 80%, 82%, 85%, 87%, 90%, 92%, 95%, 97%, 98%, 99% identical to SEQ ID NO: 5, at least 60%, 62%, 65%, 67%, 70%, 72%, 75%, 77%, 80%, 82%, 85%, 87%, 90%, 92%, 95%, 97%, 98%, 99% identical to SEQ ID NO: 6, at least 60%, 62%, 65%, 67%, 70%, 72%, 75%, 77%, 80%, 82%, 85%, 87%, 90%, 92%, 95%, 97%, 98%, 99% identical to SEQ ID NO: 7, at least 60%, 62%, 65%, 67%, 70%, 72%, 75%, 77%, 80%, 82%, 85%, 87%, 90%, 92%, 95%, 97%, 98%, 99% identical to SEQ ID NO: 8, at least 60%, 62%, 65%, 67%, 70%, 72%, 75%, 77%, 80%, 82%, 85%, 87%, 90%, 92%, 95%, 97%, 98%, 99% identical to SEQ ID NO: 9, at least 60%, 62%, 65%, 67%, 70%, 72%, 75%, 77%, 80%, 82%, 85%, 87%, 90%, 92%, 95%, 97%, 98%, 99% identical to SEQ ID NO: 10, at least 60%, 62%, 65%, 67%, 70%, 72%, 75%, 77%, 80%, 82%, 85%, 87%, 90%, 92%, 95%, 97%, 98%, 99% identical to SEQ ID NO: 11, at least 60%, 62%, 65%, 67%, 70%, 72%, 75%, 77%, 80%, 82%, 85%, 87%, 90%, 92%, 95%, 97%, 98%, 99% identical to SEQ ID NO: 12, at least 60%, 62%, 65%, 67%, 70%, 72%, 75%, 77%, 80%, 82%, 85%, 87%, 90%, 92%, 95%, 97%, 98%, 99% identical to SEQ ID NO: 13, at least 60%, 62%, 65%, 67%, 70%, 72%, 75%, 77%, 80%, 82%, 85%, 87%, 90%, 92%, 95%, 97%, 98%, 99% identical to SEQ ID NO: 14, at least 60%, 62%, 65%, 67%, 70%, 72%, 75%, 77%, 80%, 82%, 85%, 87%, 90%, 92%, 95%, 97%, 98%, 99% identical to SEQ ID NO: 15, at least 60%, 62%, 65%, 67%, 70%, 72%, 75%, 77%, 80%, 82%, 85%, 87%, 90%, 92%, 95%, 97%, 98%, 99% identical to SEQ ID NO: 16, at least 60%, 62%, 65%, 67%, 70%, 72%, 75%, 77%, 80%, 82%, 85%, 87%, 90%, 92%, 95%, 97%, 98%, 99% identical to SEQ ID NO: 17, or at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16.

In some embodiments, the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

In some embodiments, the antibody or functional fragment thereof comprises a heavy chain comprising an amino acid sequence identical to SEQ ID NO: 1 and a light chain comprising an amino acid sequence identical to SEQ ID NO: 2. In some embodiments, the antibody or functional fragment thereof comprises a heavy chain comprising an amino acid sequence identical to SEQ ID NO: 3 and a light chain comprising an amino acid sequence identical to SEQ ID NO: 4. In some embodiments, the antibody or functional fragment thereof comprises a heavy chain comprising an amino acid sequence identical to SEQ ID NO: 5 and a light chain comprising an amino acid sequence identical to SEQ ID NO: 6. In some embodiments, the antibody or functional fragment thereof comprises a heavy chain comprising an amino acid sequence identical to SEQ ID NO: 7 and a light chain comprising an amino acid sequence identical to SEQ ID NO: 8. In some embodiments, the antibody or functional fragment thereof comprises a heavy chain comprising an amino acid sequence identical to SEQ ID NO: 9 and a light chain comprising an amino acid sequence identical to SEQ ID NO: 10. In some embodiments, the antibody or functional fragment thereof comprises a heavy chain comprising an amino acid sequence identical to SEQ ID NO: 11 and a light chain comprising an amino acid sequence identical to SEQ ID NO: 12. In some embodiments, the antibody or functional fragment thereof comprises a heavy chain comprising an amino acid sequence identical to SEQ ID NO: 13 and a light chain comprising an amino acid sequence identical to SEQ ID NO: 14. In some embodiments, the antibody or functional fragment thereof comprises a heavy chain comprising an amino acid sequence identical to SEQ ID NO: 15 and a light chain comprising an amino acid sequence identical to SEQ ID NO: 16. In some embodiments, the antibody or functional fragment thereof comprises a heavy chain comprising an amino acid sequence identical to SEQ ID NO: 17 and a light chain comprising an amino acid sequence identical to SEQ ID NO: 18.

In some embodiments, the antibody or functional fragment thereof comprises a heavy chain comprising an amino acid sequence comprising the complementarity-determining regions (CDRs) of SEQ ID NO: 1 and a light chain comprising an amino acid sequence comprising the complementarity-determining regions (CDRs) of SEQ ID NO: 2. In some embodiments, the antibody or functional fragment thereof comprises a heavy chain comprising an amino acid sequence comprising the complementarity-determining regions (CDRs) of SEQ ID NO: 3 and a light chain comprising an amino acid sequence comprising the complementarity-determining regions (CDRs) of SEQ ID NO: 4. In some embodiments, the antibody or functional fragment thereof comprises a heavy chain comprising an amino acid sequence comprising the complementarity-determining regions (CDRs) of SEQ ID NO: 5 and a light chain comprising an amino acid sequence comprising the complementarity-determining regions (CDRs) of SEQ ID NO: 6. In some embodiments, the antibody or functional fragment thereof comprises a heavy chain comprising an amino acid sequence comprising the complementarity-determining regions (CDRs) of SEQ ID NO: 7 and a light chain comprising an amino acid sequence comprising the complementarity-determining regions (CDRs) of SEQ ID NO: 8. In some embodiments, the antibody or functional fragment thereof comprises a heavy chain comprising an amino acid sequence comprising the complementarity-determining regions (CDRs) of SEQ ID NO: 9 and a light chain comprising an amino acid sequence comprising the complementarity-determining regions (CDRs) of SEQ ID NO: 10. In some embodiments, the antibody or functional fragment thereof comprises a heavy chain comprising an amino acid sequence comprising the complementarity-determining regions (CDRs) of SEQ ID NO: 11 and a light chain comprising an amino acid sequence comprising the complementarity-determining regions (CDRs) of SEQ ID NO: 12. In some embodiments, the antibody or functional fragment thereof comprises a heavy chain comprising an amino acid sequence comprising the complementarity-determining regions (CDRs) of SEQ ID NO: 13 and a light chain comprising an amino acid sequence comprising the complementarity-determining regions (CDRs) of SEQ ID NO: 14. In some embodiments, the antibody or functional fragment thereof comprises a heavy chain comprising an amino acid sequence comprising the complementarity-determining regions (CDRs) of SEQ ID NO: 15 and a light chain comprising an amino acid sequence comprising the complementarity-determining regions (CDRs) of SEQ ID NO: 16. In some embodiments, the antibody or functional fragment thereof comprises a heavy chain comprising an amino acid sequence comprising the complementarity-determining regions (CDRs) of SEQ ID NO: 17 and a light chain comprising an amino acid sequence comprising the complementarity-determining regions (CDRs) of SEQ ID NO: 18.

In some embodiments, the virus is a coronavirus. In some embodiments, the coronavirus is a coronavirus variant. In some embodiments, the coronavirus is a coronavirus-related virus. In some embodiments, the coronavirus is a wildtype SARS-COV-2 (D614G) virus. In some embodiments, the coronavirus is a SARS-COV-2 B.1.1.7 variant virus. In some embodiments, the coronavirus is a SARS-COV-2 B.1.351 variant virus. In some embodiments, the coronavirus is a SARS-COV-2 P.1 variant virus. In some embodiments, the coronavirus is a SARS-COV-2 B.1.526 variant virus. In some embodiments, the coronavirus is a SARS-COV-2 B.1.429 variant virus. In some embodiments, the coronavirus is a SARS-CoV-2 B.1.617.1v1 variant virus. In some embodiments, the coronavirus is a SARS-COV-2 B.1.617.2 variant virus. In some embodiments, the coronavirus is a SARS-COV-2 B.1.1.1 variant virus. In some embodiments, the coronavirus is a SARS-COV-2 D614G virus. In some embodiments, the coronavirus is a SARS-COV-2 C.37 variant virus. In some embodiments, the coronavirus is a SARS-COV-2 Omicron (B.1.1.529) variant virus. In some embodiments, the coronavirus is a SARS-COV-2 Omicron BA.1 (B.1.1.529.1) variant virus. In some embodiments, the coronavirus is a SARS-COV-2 Omicron BA.1.1 (B.1.1.529.1.1) variant virus. In some embodiments, the coronavirus is a SARS-COV-2 Omicron BA.2 (B.1.1.529.2) variant virus. In some embodiments, the coronavirus is a SARS-COV-2 B.1.621 variant virus. In some embodiments, the coronavirus is a SARS-COV-2 WA1 virus. In some embodiments, the coronavirus is any virus of Table 1. In some embodiments, the coronavirus is a SARS-CoV virus.

In some embodiments, the at least a portion of a virus comprises a coronavirus spike protein. In some embodiments, the at least a portion of a virus comprises a juncture between a subdomain 1 (SD1) and a N-Terminal Domain (NTD) of a coronavirus spike protein. In some embodiments, the at least a portion of a virus comprises a subdomain 1 (SD1) of a coronavirus spike protein. In some embodiments, the at least a portion of a virus comprises a N-Terminal Domain (NTD) of a coronavirus spike protein.

In certain aspects the subject matter described herein provides a polynucleotide encoding an antibody or a fragment thereof, wherein the antibody or fragment thereof comprises any one of SEQ ID NOs: 1-18.

In certain aspects the subject matter described herein provides a vector comprising a polynucleotide encoding an antibody or a fragment thereof, wherein the antibody or fragment thereof comprises any one of SEQ ID NOs: 1-18.

TABLE 1
SARS-CoV-2 variants and descendent lineage

| | |
|---|---|
| Alpha | B.1.1.7 and Q lineages |
| Beta | B.1.351 and descendent lineages |
| Delta | B.1.617.2 and AY lineages |
| Gamma | P.1 and descendent lineages |
| Epsilon | B.1.427 and B.1.429 |
| Eta | B.1.525 |
| Iota | B.1.526 |
| Kappa | B.1.617.1 |
| N/A | 1.617.3 |
| Mu | B.1.621, B.1.621.1 |
| Omicron | B.1.1.529 and BA lineages |
| Zeta | P.2 |

### Antibody Sequences

Underlined and italicized amino acids represent the respective complementarity-determining regions (CDRs).

Antibodies binding the receptor binding domain (RBD) of the coronavirus spike(S) protein, which engages with the receptor angiotensin-converting enzyme 2 (ACE2) on host cells.

SEQ ID NO: 1 is the variable region of the heavy chain of antibody number
10-16.
QMQLVQSGPEVKKPGTSVKVSCKASGFTFISSAMQWVRQARGQRLEWIGWIVVGSG
NTNYAQKFQERVSITRDMSTSTAYMELSSLRSEDTAVYYCAAPYCNSTNCYDAFNIWG
QGTMVTVSS
SEQ ID NO: 2 is the variable region of the light chain of antibody number 10-16.
EIVLTQSPGTLSLSPGERATLSCRASQSVSSSYLAWYQQKPGQAPRLLIYGASSRATGIP
DRFSGSGSGTDFTLTISRLEPEDFAVYYCQQYGRSPWTFGQGTKVDIK
SEQ ID NO: 3 is the variable region of the heavy chain of antibody number
10-40.
QLQLQESGPGLVKPSETLSLTCTVSGGSISSSNFYWGWIRQPPGKGLEWIASITYSGRTF
YNPSLKSRVAISVDTSKNQFSLKLSSVTAADTAVYYCARTFPSYYDRSGYHYLNYGMD
VWGQGTTVTVSS
SEQ ID NO: 4 is the variable region of the light chain of antibody number 10-40.
NFMLTQPHSMSESPGKTVTISCTRSSGSIASNYVQWYQQRPGSSPTTVIYEDNQRPSGV
PDRFSGSIDSSSNSASLTISGLKTEDEADYYCQSYDSSSWVFGGGTKLTVL
SEQ ID NO: 5 is the variable region of the heavy chain of antibody number
11-11.
QVQLQESGPGLVKPSQTLSLTCTVSGGSISSGGYYWSWIRQHPGKGLEWIGYIYYSGST
YYNPSLKSRVTISVDTSKNQFSLKVSSVTAADTAVYYCASSNYYDGSGYNLLESYGMD
VWGQGTSVTVSS
SEQ ID NO: 6 is the variable region of the light chain of antibody number 11-11.
QSVLTQPPSVSGAPGQRVTISCTGSSSNIGAGYDVHWYQQLPGTAPKLLIYGNSNRPSG
VPDRFSGSRSGTSASLAITGLQAEDEADYYCQSYDSSLSGSWVFGGGTKVTVL
SEQ ID NO: 7 is the variable region of the heavy chain of antibody number 12-6.
QVQLVQSGAEVKKPGSSVKVSCKASGGTFSNYAISWVRQAPGQGLEWMGGFIRIFDT
AIYAQKFQGRVTITADESTSTAYMDLSSLRSEDTAVYYCARSPYYYDKSGYYYFDHWG
QGTLVTVSS
SEQ ID NO: 8 is the variable region of the light chain of antibody number 12-6.
DIQMTQSPSSLSASVGDRVTITCRASQNISSYLNWYQQKPGKAPNLLIYAASSLQSGVP
SRFSGSGSGTDFTLTISSLQPEDFATYYCQQSYSPQWTFGQGTKVEIK
SEQ ID NO: 9 is the variable region of the heavy chain of antibody number
12-27.
EVQLVQSGAEVKKPGESLKMSCKGSGYNFTFYWIGWVRQMPGKGLEWMGSIYPGDS
ETRYSPSFQGQVTISAATSISTAYLQWSSLKASDAAMYYCTARLSGVQTALEYFDFWG
QGTLVTVSS
SEQ ID NO: 10 is the variable region of the light chain of antibody number 12-27.
DIQMTQSPSSLSASVGDRVTITCQASQDITNHLNWYQLRPGKAPKLLIYDASNLDAGV
PSRFSGSGSGTDFTFTISSLQPEDIATYYCQQYDNLLLTFGGGTKVEIK
SEQ ID NO: 13 is the variable region of the heavy chain of antibody number
10-28.
QVQLVESGGGVVQPGRSLRLSCAASGFTFSSYDMHWVRQAPGKGLEWVAVISYDGS
SKFYAESVKGRFTISRDNSKNTLYLQMNSLRAEETAVYYCVKDGEQLVPLFDYWGQG
TLVTVSS
SEQ ID NO: 14 is the variable region of the light chain of antibody number 10-28.
DIQMTQSPSSLSASVGDRVTITCRASQSISSYLNWYQQKPGKAPKLLIYAASSLQSGVP
SRFSGSGSGTDFTLTISSLQPEDFATYYCQQSYSTPGVTFGPGTKVDIK

Antibodies biding the N-terminal domain (NTD) of the S protein.

SEQ ID NO: 11 is the variable region of the heavy chain of antibody number
13-2.
QVQLVESGGGVVQPGRSLRLSCAASGFIFSDFGMHWVRQAPGKGLEWVAVISYDGS
NKYYADSVKGRFTISRDNSKNTLYLQMNSLRAEDTAVYYCKARDYYDISGFYHPPNF
DYWGQGTLVTVSS
SEQ ID NO: 12 is the variable region of the light chain of antibody number 13-2.
SYELTQPPSVSVSPGQTARIPCSGHTLPKQYAYWYQQKPGQAPVLVIYKDSERPSGIPE
RFSGSSSGTTVTLTISGVQAEDEANYYCQSADSSGTYWVFGGGTKLTVL

In some embodiments, the subject matter described herein relates to engineering mAbs. In some embodiments, the engineered mAbs express the immunoglobulin variable regions of mAbs 2-16 or 12-19, which have been isolated from human patents infected with a SARS-COV-2 virus.

Underlined and italicized amino acid sequences represent the respective complementarity-determining regions (CDRs).

SEQ ID NO: 15 is the variable region of the heavy chain of antibody number
12-16.
QVQLVESGGGVVQPGRSLRLSCAASGFTFSNYGMHWVRQAPGKGLEWVALISFDGS
NRYYADSVKGRFTISRDNSKNTLYLQMDSLRAEDTAVYYCAKDLSFYYDISTGYYPQS
YNSGTDVWGQGTTVTVSS
SEQ ID NO: 16 is the variable region of the light chain of antibody number 12-16.
SYELTQPPSVSVSPGQTASITCSGDKLGDKYACWYQQKPGQSPVLVIYQDNKRPSGIP
ERFSGSNSGNTATLTISGTQAMDEADYYCQGWDRSTGYYVFGTGTKVTVL
SEQ ID NO: 17 is the variable region of the heavy chain of antibody number
12-19.
QVQLVESGGGVVQPGRSLRLSCEASGFTFNTYDMHWARQAPGKGLEWVAVIWYDG
SNKFYADSVKGRFTISRDNSKNTLYLQMNSLRAEDTAVYYCARDRTPVYDILTGYYWP
PPRVDGMDVWGQGTTVTVSS
SEQ ID NO: 18 is the variable region of the light chain of antibody number 12-19.
DNVMTQTPFSLSVTPGQPASISCKSSQSLLHSDGKTYLYWYLQKPGQSPQLLIYEVSNR
FSGVPERFSGSGSGTDFTLKISRVEAEDVGVYYCMQSIQLPFTFGQGTKLEIK

### Antibody Therapy

In some embodiments, antibody therapy utilizes monoclonal antibodies, which recognize and specifically bind to pre-determined antigens. In some embodiments, antibody administration to a subject stimulates that subject's immune system to recognize and attack those antigens such as viruses. In some embodiments, the route of administration of therapeutic antibodies to subjects is through intravenous (IV) infusion. In some embodiments, the route of administration of therapeutic antibodies to subjects is through subcutaneous injection. In some embodiments, the route of administration of therapeutic antibodies to subjects is through intramuscular injection. In some embodiments, administration of a therapeutic antibody or a cocktail thereof can be achieved through a combination of all three administration routs. In some embodiments, oral administration routes of therapeutic antibodies are also possible. In some embodiments, the engineered monoclonal antibodies described herein can be administered to a subject through any useful method known in the art. In some embodiments, the engineered mAbs described herein or a cocktail thereof are administered daily. In some embodiments, the engineered mAbs described herein are administered twice, three times, four times, five times, or several times a day. In some embodiments, the engineered mAbs described herein are administered for a total of 1, 2, 3, 4, 5, 6 days or for 1 or 2 weeks. In some embodiments, the engineered mAbs described herein are administered for a period longer than two weeks. In some embodiments, the engineered mAbs described herein are administered in combination with any therapeutically effective agent or a combination of agents known in the art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

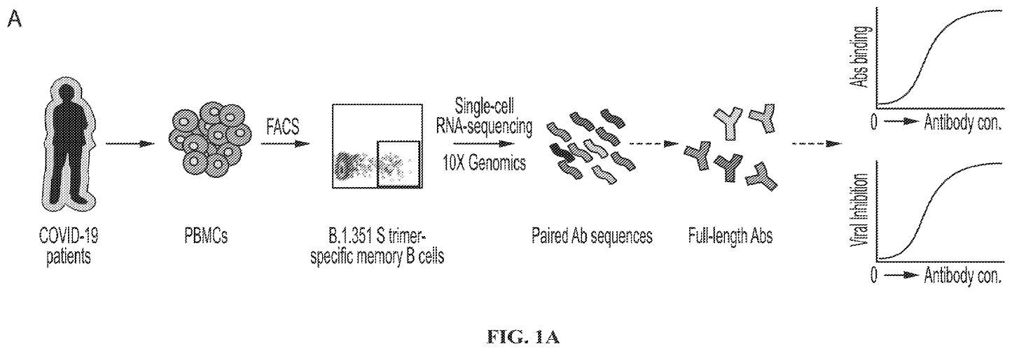

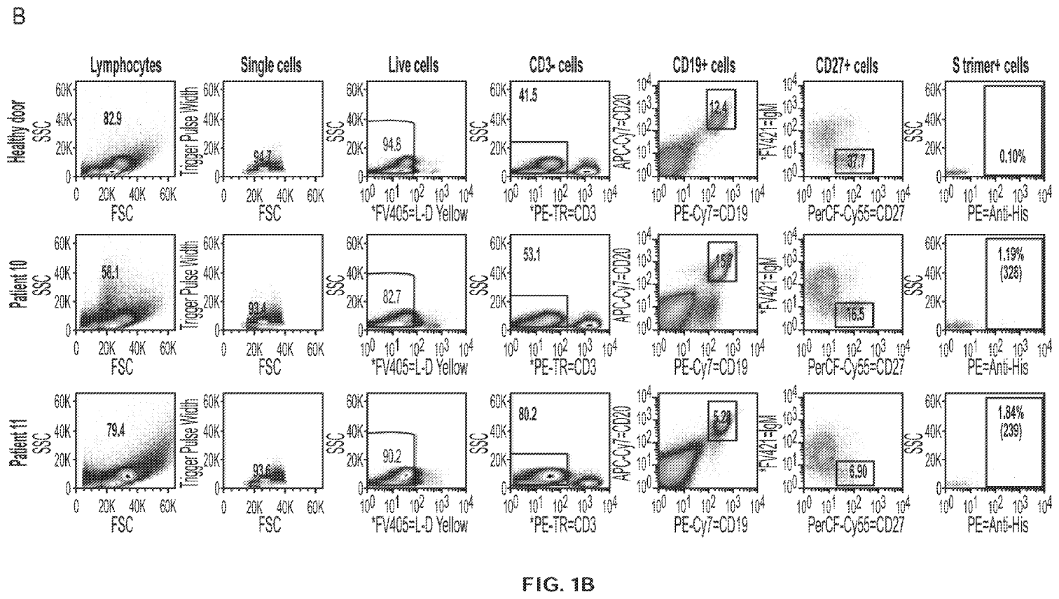

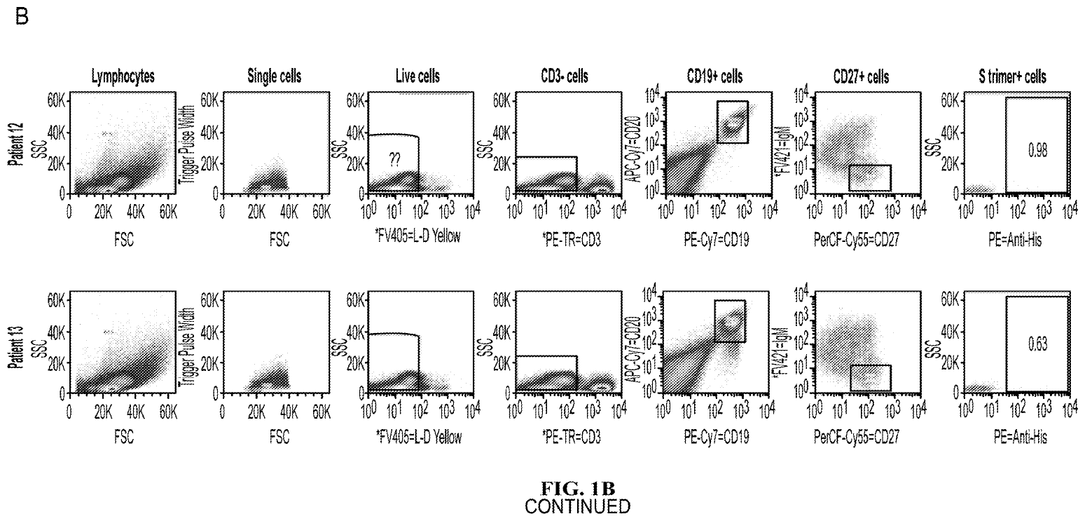

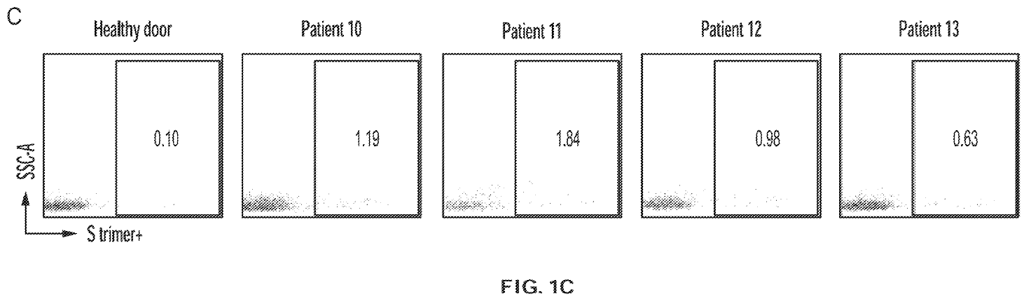

FIGS. 1A-C show SARS-COV-2 S trimer-specific antibody isolation. Biological samples from vaccinated or unvaccinated patients infected with SARS-COV-2 or its variants are isolated. In one embodiment, the biological sample is plasma. The samples are then evaluated for the ability of potential antibodies contained within these samples to neutralize SARS-COV-2 and various variants of concern. Peripheral blood mononuclear cells (PMBCs) are isolated from those patients that exhibited the strongest activity in order to isolate S trimer-specific memory B cells. The sequences encoding the antibodies are recovered from these single B cells. In some embodiments, the recovery can be performed using high throughput sequencing. Genes from single B cells encoding for these mAb sequences are engineered and cloned into expression vectors. mAbs are expressed in vitro and purified for subsequent analyses and characterization. In some embodiments, the sequences are modified or optimized. The engineered, in vitro-produced and purified, mAbs are then tested for their ability to neutralize SARS-COV-2, its variants (by both pseudotyped virus assay and live virus assay), and related coronaviruses. Using this methodology, engineered monoclonal antibodies have been identified as top hits with antiviral activity against SARS-COV-2, multiple VOC and VOI, and related coronaviruses. In some embodiments, the engineered monoclonal antibodies described herein have been identified as having strong antiviral activity and potency against other SARS-like coronaviruses. FIG. 1A shows an illustrative representation of SARS-COV-2 S trimer-specific antibody isolation strategy. FIG. 1B shows sorting of cells from patients and a healthy donor. FIG. 1C shows SSC-A flow cytometry using the S trimer of the SARS-COV-2 virus.

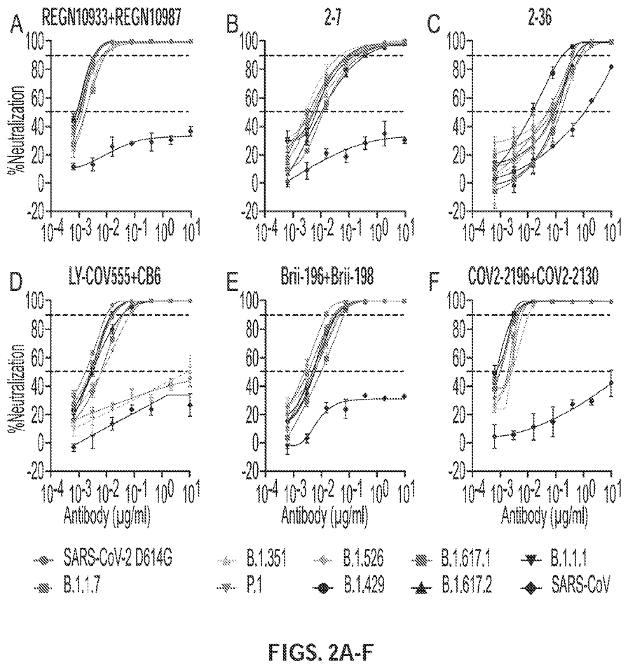

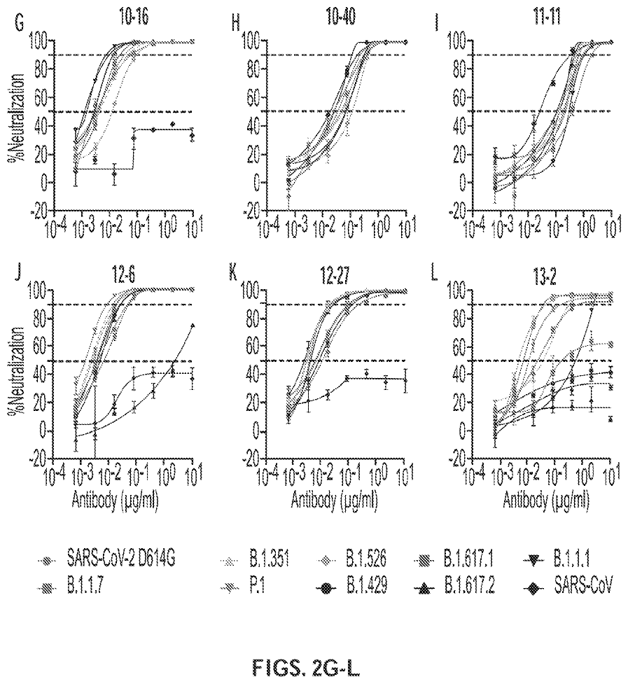

FIGS. 2A-L show neutralization profiles of down-selected engineered monoclonal antibodies on wild-type coronavirus and variants of concern PVs. In FIGS. 2A-F, six different previously reported monoclonal antibodies or antibody combinations have been used as controls in the assay. In FIGS. 2G-L, six down-selected engineered monoclonal antibodies were purified from transfection supernatants and further characterized for their neutralizing activity against the SARS-COV-2 Wuhan-Hu-1 strain carrying the D614G mutation, eight other variants of concern, B.1.1.7, B.1.351, P.1, B.1.526, B.1.429, B.1.617.1, B.1.617.2, and B.1.1.1, and SARS-COV. Fivefold serial dilutions of engineered monoclonal antibodies from 10 μg/ml to 0.64 ng/ml were pre-incubated with pseudoviruses with coronavirus spike proteins and added to Vero E6 cells. FIG. 2A shows mAbs REGN10933 and REGN10987. FIG. 2B shows mAb 2-7. FIG. 2C shows mAb 2-36. FIG. 2D shows mAbs LY-COV555 and CB6. FIG. 2E shows mAb Brii-196 and Brii-198. FIG. 2F shows mAbs COV2-2196 and COV2-2130. FIG. 2G shows the neutralization profile of engineered monoclonal antibody 10-16. FIG. 2H shows the neutralization profile of engineered monoclonal antibody 10-40. FIG. 2I shows the neutralization profile of engineered monoclonal antibody 11-11. FIG. 2J shows the neutralization profile of engineered monoclonal antibody 12-6. FIG. 2K shows the neutralization profile of engineered monoclonal antibody 12-27. FIG. 2L shows the neutralization profile of engineered monoclonal antibody 13-2.

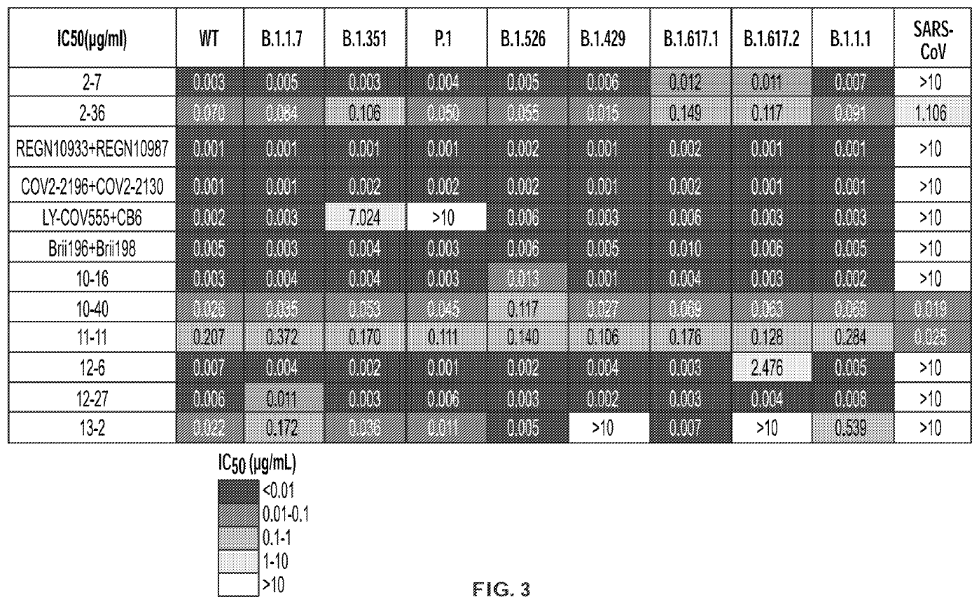

FIG. 3 shows the IC₅₀ (μg/ml) values from neutralization of SARS-COV-2 and SARS-COV pseudoviruses with down-selected engineered monoclonal antibodies. Among the six down-selected antibodies, four antibodies, 10-16, 11-11, 10-40, and 12-27 showed broad and potent neutralizing activities against multiple SARS-COV-2 spike protein variants of concern with IC₅₀ values ranging from 1 ng/ml to 284 ng/ml. Engineered monoclonal antibody 12-6 also exhibited potent neutralizing activity with IC₅₀ values ranging from 1 ng/ml to 5 ng/ml against seven variants including B.1.1.7, B.1.351, P.1, B.1.429, B.1.526, B.1.617.1, and B.1.1.1. Engineered monoclonal antibody 13-2 exhibited antiviral efficacy with IC₅₀ values ranging from 5 ng/ml to 539 ng/ml against six variants including B.1.1.7, B.1.351, P.1, B.1.526, B.1.617.1, and B.1.1.1. Notably, two engineered monoclonal antibodies, 10-40 and 11-11, display remarkable cross-reactivity against SARS-COV with IC₅₀ values 19 ng/ml to 25 ng/ml, respectively.

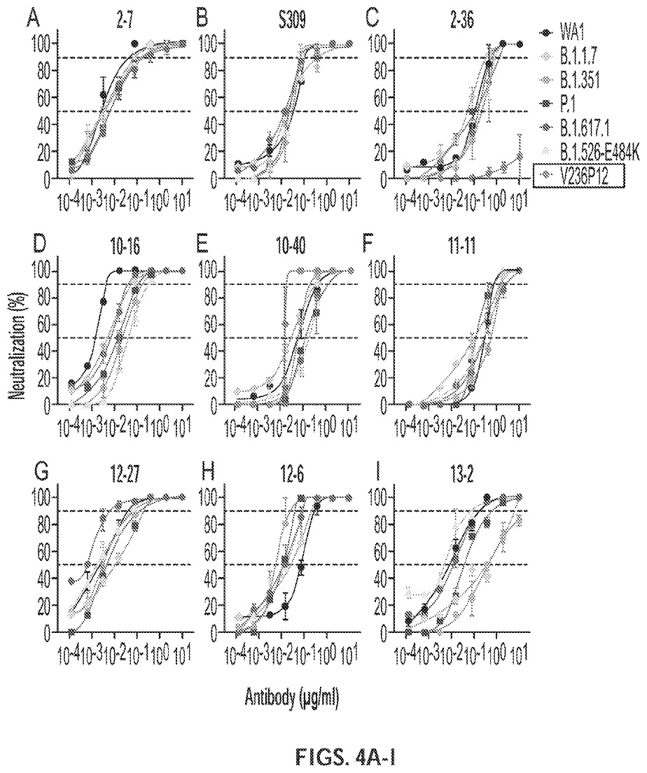

FIGS. 4A-J show neutralization of authentic SARS-COV-2 and 6 of its variants. In FIGS. 4A-C, three different reported monoclonal antibodies have been used as controls in the assay. In FIGS. 4D-I, six down-selected engineered monoclonal antibodies were purified from transfection supernatants and further characterized for their neutralizing activity against USA-WA1/2020 strain, five current variants of concern, B.1.1.7, B.1.351, P.1, B.1.526, and B.1.617.1, and V236P12 (mAb 2-36 resistant virus). Fivefold serial dilutions of engineered monoclonal antibodies from 10 μg/ml to 0.64 ng/ml were pre-incubated with authentic coronaviruses and added to Vero E6 cells. FIG. 4A shows neutralization with mAb 2-7. FIG. 4B shows neutralization with mAb S309. FIG. 4C shows neutralization with mAb 2-36. FIG. 4D shows neutralization with engineered mAb 10-16. FIG. 4E shows neutralization with engineered mAb 10-40. FIG. 4F shows neutralization with engineered mAb 11-11. FIG. 4G shows neutralization with engineered mAb 12-27. FIG. 4H shows neutralization with engineered mAb 12-6. FIG. 4I shows neutralization with engineered mAb 13-2. FIG. 4J shows potency with IC₅₀ values of various monoclonal antibodies. Among the six down-selected antibodies, five engineered antibodies, 10-16, 11-11, 10-40, 12-6, and 12-27 showed broad and potent neutralizing activities against five variants of concern, B.1.1.7, B.1.351, P.1, B.1.526, B.1.167.1 with IC₅₀ values ranging from 1 ng/ml to 550 ng/ml. Engineered mAb 13-2 showed antiviral efficacy with IC₅₀ values ranging from 5 ng/ml to 38 ng/ml against three variants including P.1, B.1.526, and B.1.617.1. Notably, engineered mAb 10-40 inhibits V236P12 (a 2-36 resistant virus) with an IC₅₀ value of 15 ng/ml.

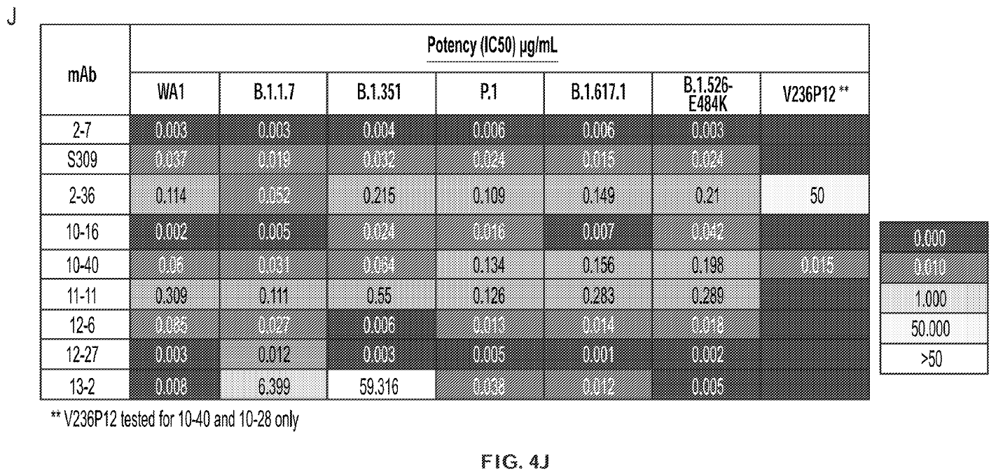

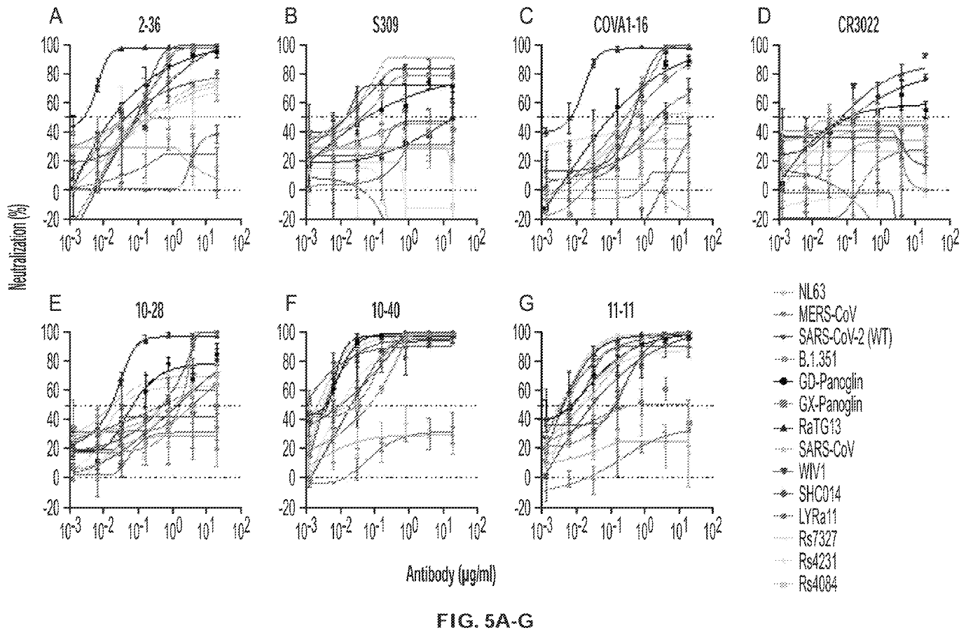

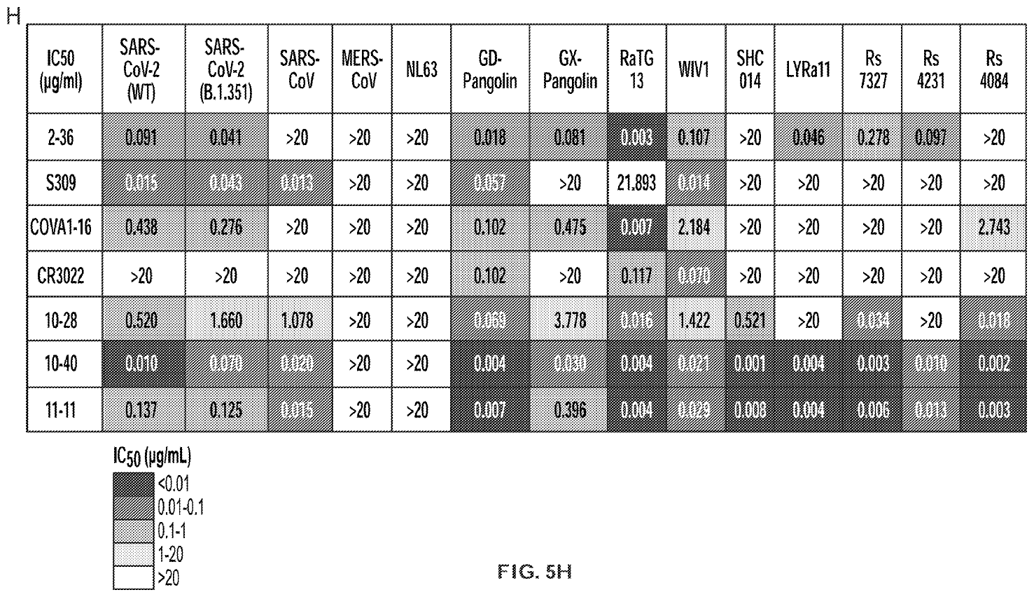

FIGS. 5A-H show the breadth and potency of engineered monoclonal antibodies 10-28, 10-40, and 11-11 against SARS-like coronaviruses, which utilize the human ACE2 receptor to invade a host cell, in comparison with other monoclonal antibodies. In FIGS. 5A-D, four different previously reported monoclonal antibodies have been used as controls in the assay. In FIGS. 5E-G, 3 down-selected engineered monoclonal antibodies, 10-28, 10-40, and 11-11 were purified from transfection supernatants and further characterized for their neutralizing activity against different pseudotyped viruses including Wuhan-Hu-1 strain, B.1.351 variant, SARS-COV and human ACE2-utilizing sarbecoviruses. Fivefold serial dilutions of engineered monoclonal antibodies from 10 μg/ml to 0.64 ng/ml were pre-incubated with pseudotyped viruses and added to 293T-hACE2 cells. FIG. 5A shows monoclonal antibody 2-36. FIG. 5B shows monoclonal antibody S309. FIG. 5C shows monoclonal antibody COVA1-16. FIG. 5D shows monoclonal antibody CR3022. FIG. 5E shows engineered monoclonal antibody 10-28. FIG. 5F shows engineered monoclonal antibody 10-40. FIG. 5G shows engineered monoclonal antibody 11-11. FIG. 5H shows potency of various engineered monoclonal antibodies. Engineered monoclonal antibody 10-28 showed broad and potent neutralizing activities against human ACE2-utilizing sarbecoviruses, GD-Pangolin, GX-Pangolin, RaTG13, WIV1, SHC014, Rs7327, and Rs4084 with IC₅₀ values ranging from 16 ng/ml to 521 ng/ml. Engineered monoclonal antibody 10-11 showed broad and potent neutralizing activities against all human ACE2-utilizing sarbecoviruses tested, GD-Pangolin, GX-Pangolin, RaTG13, WIV1, SHC014, LYRa11, Rs7327, Rs4231, and Rs4084 with IC₅₀ values ranging from 2 ng/ml to 30 ng/ml and with IC₉₀ values ranging from 27 ng/ml to 855 ng/ml. Engineered monoclonal antibody 11-11 showed broad and potent neutralizing activities against all human ACE2-utilizing sarbecoviruses tested, GD-Pangolin, GX-Pangolin, RaTG13, WIV1, SHC014, LYRa11, Rs7327, Rs4231, and Rs4084 with IC₅₀ values ranging from 3 ng/ml to 396 ng/ml.

### Example 2-Identification and Comparative Analysis of Antibodies that Broadly Neutralize Sarbecoviruses

In some embodiments, the subject matter disclosed herein relates to three engineered monoclonal antibodies, 10-28, 10-40, and 11-11, with broad neutralization activity against sarbecoviruses. In some embodiments, the subject matter described herein relates to the isolation and characterization of engineered mAbs 10-28, 10-40, and 11-11. In some embodiments, the subject matter disclosed herein relates to a comprehensive comparison of the three engineered monoclonal antibodies, 10-28, 10-40, and 11-11, with other RBD-directed broadly neutralizing antibodies. In some embodiments, engineered monoclonal antibody 10-40 neutralizes or binds all sarbecoviruses tested herein. In some embodiments, the subject matter disclosed herein relates to structural analyses highlighting a common well-conserved epitope in the RBD of the coronavirus spike proteins, which is specifically recognized by these broadly neutralizing engineered antibodies. In some embodiments, engineered antibody 10-40 and other broad antibodies share a common paratope, with a specific motif in the CDRH3 region. In some embodiments, engineered antibody 10-40 displays protection against SARS-COV-2 in a mouse model of the infection.

In some embodiments of the subject matter described herein, antibodies were isolated from blood samples from patients infected with SARS-COV-2 followed by vaccination or infected by SARS-COV-2 variant(s) harboring the E484K mutation. Once the antibody sequences were obtained from these samples, the monoclonal antibodies disclosed herein were engineered in vitro and used for subsequent characterization experiments. The top mAbs hits identified herein show exceptional potency and breadth against original SARS-CoV-2, multiple variants of concern (VOC) and variants of interest (VOI), and related coronaviruses. In some embodiments, the engineered antibodies disclosed herein demonstrate significant potency and breadth against SARS-COV-1 and other sarbecoviruses.

In some embodiments, the engineered neutralizing mAbs disclosed herein can be used for treatment of patients infected with SARS-COV-2, its variants, as well as future SARS-like coronavirus infections. In some embodiments, the engineered monoclonal antibodies disclosed herein reduce coronavirus viral load and/or disease severity in patients. In some embodiments, the engineered antibodies described herein ultimately improve patients' clinical outcomes. The engineered mAbs described herein can also be used as prophylaxis to prevent high risk individuals from becoming infected with SARS-COV-2 variants. These mAbs could also potentially be used as prophylaxis for the general population to prepare future outbreak(s) caused by SARS-COV-2 variants or SARS like coronaviruses.

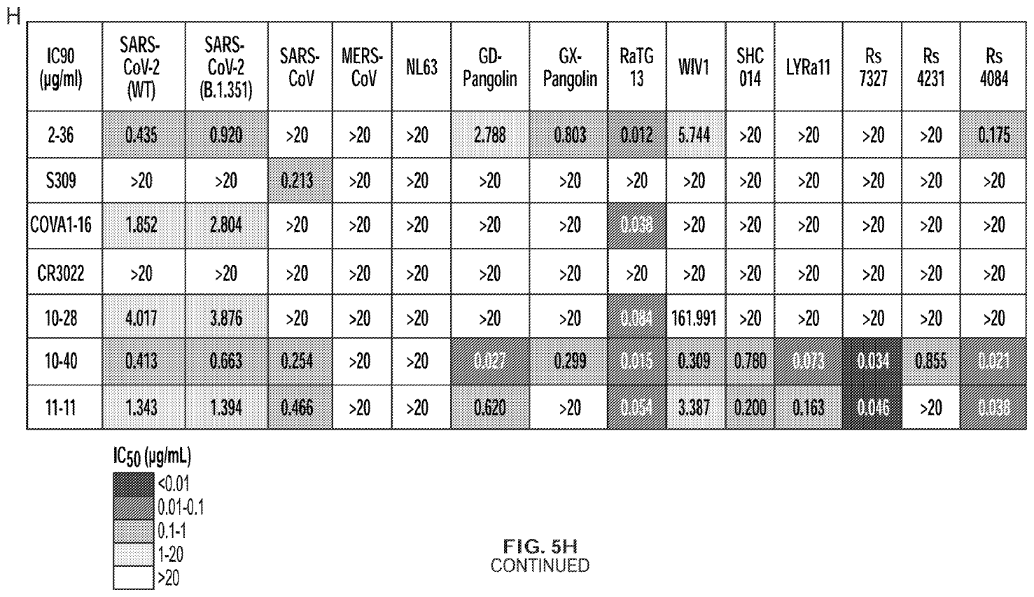

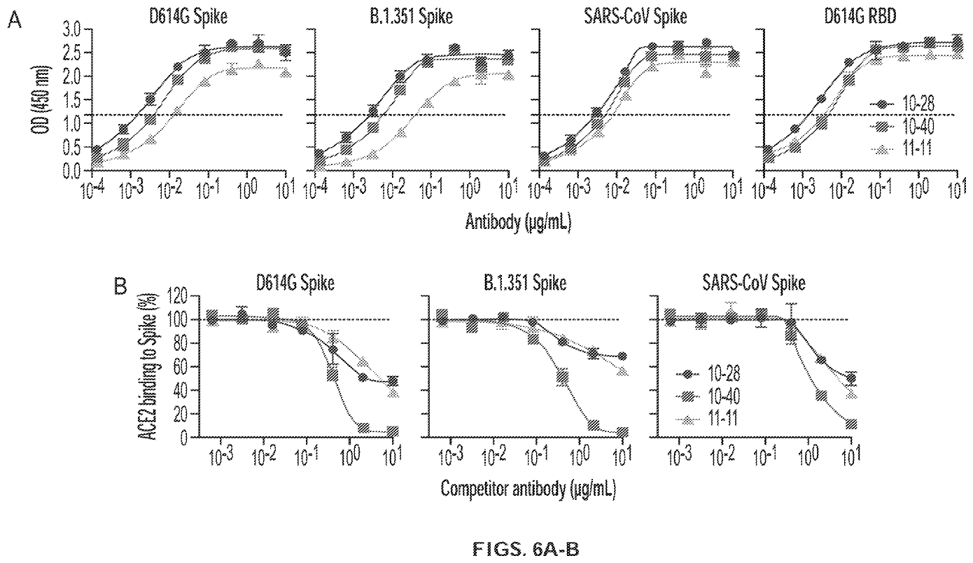

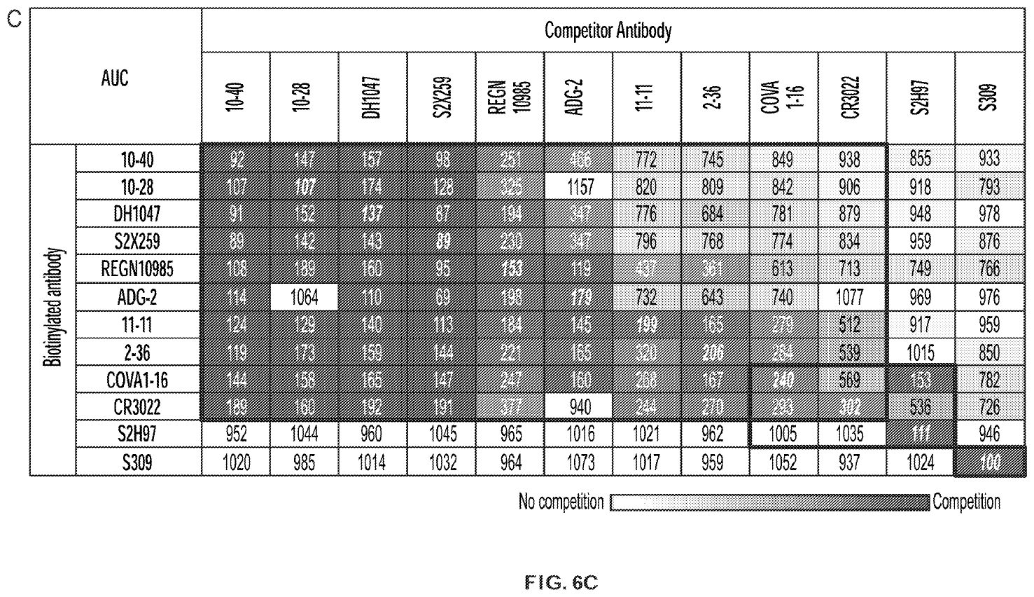

FIGS. 6A-C show identification of three broadly neutralizing sarbecovirus engineered monoclonal antibodies-10-28, 10-40, and 11-11. FIG. 6A shows that antibodies were tested for binding to the spike trimers of SARS-COV-2 D614G, B.1.351, SARS-COV, as well as the RBD of D614G by ELISA. Data are shown as mean±SD of two technical replicates. FIG. 6B shows inhibition of ACE2 binding to SARS-COV-2 D614G, B.1.351, and SARS-COV spike trimers by engineered antibodies 10-28, 10-40, and 11-11. Testing was performed by ELISA. Data are shown as mean±SD of two technical replicates. FIG. 6C shows epitope mapping by competition ELISA of RBD-directed broadly neutralizing antibodies. Select antibodies were tested for blocking binding of biotinylated monoclonal antibodies to SARS-COV-2 D614G spike trimer.

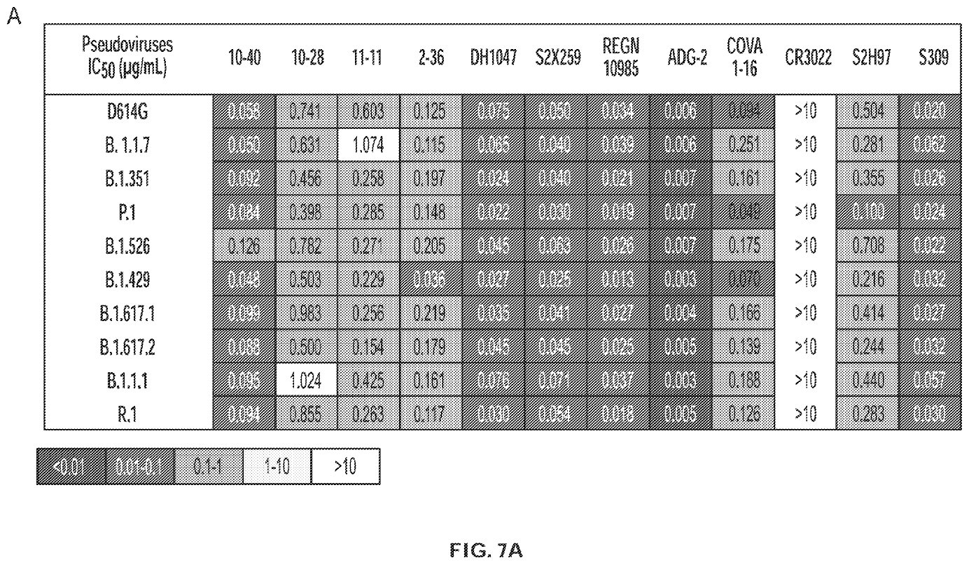

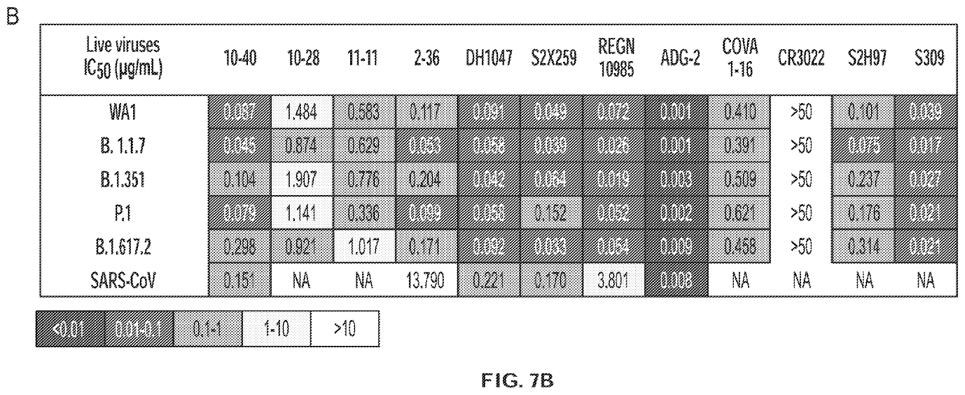

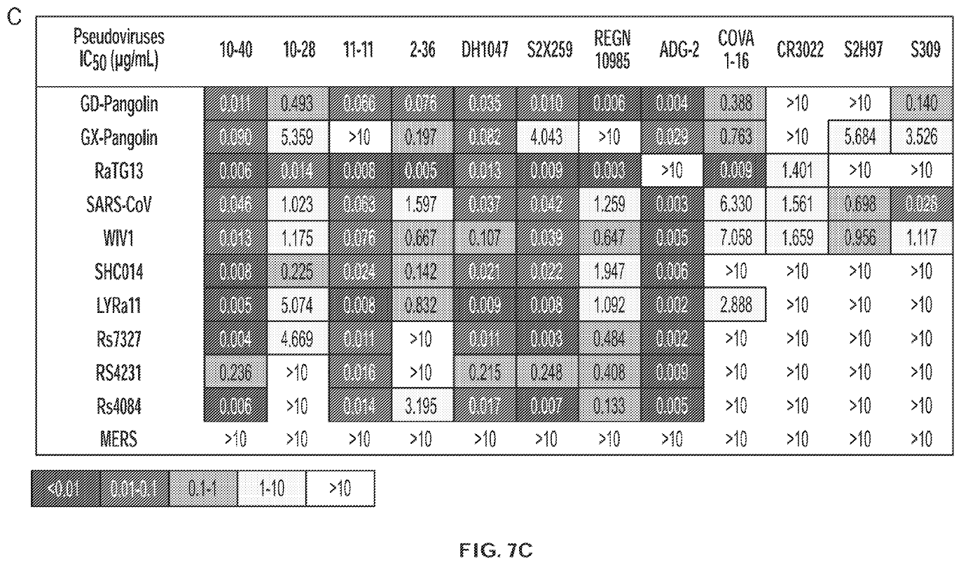

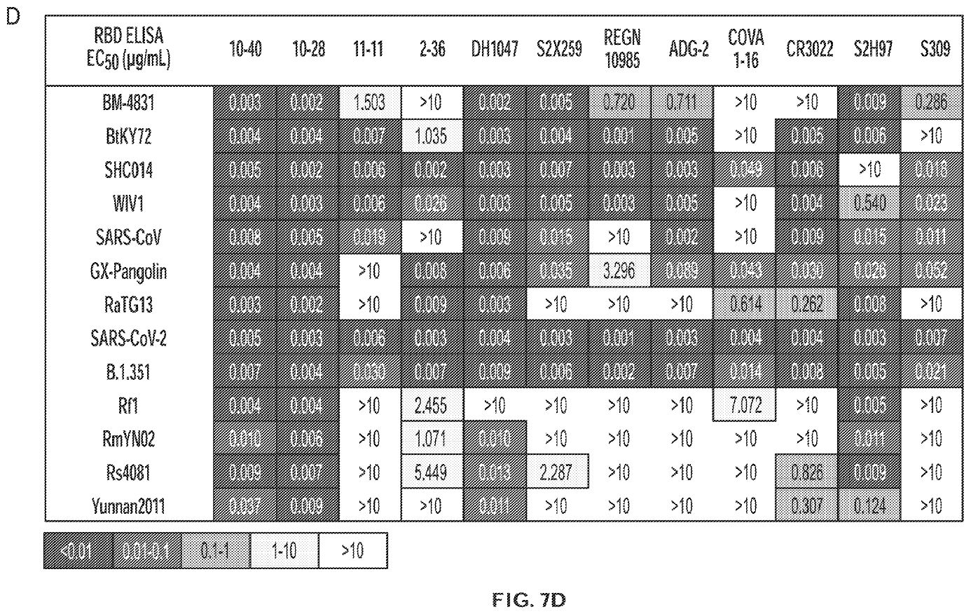

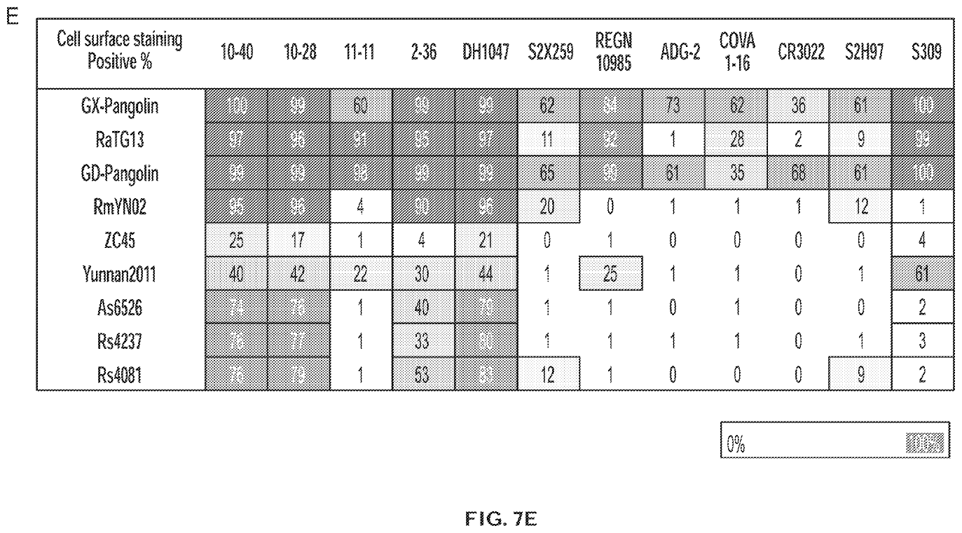

FIGS. 7A-E show breadth and potency of engineered monoclonal antibodies 10-40, 10-28, and 11-11. FIG. 7A shows neutralization titers (IC₅₀) of selected monoclonal antibodies against VSV pseudotypes with SARS-COV-2 D614G and variant spikes. FIG. 7B shows neutralization titers (IC₅₀) of selected monoclonal antibodies against authentic SARS-CoV-2 and SARS-COV strains in a cytopathic effect reduction assay. FIG. 7C shows neutralization titers (IC₅₀) of selected monoclonal antibodies against VSV pseudotypes with sarbecovirus spikes. FIG. 7D shows binding titers (EC₅₀) of selected monoclonal antibodies to purified RBD proteins, representative of the different sarbecovirus clades, and SARS-CoV-2 variants as measured by ELISA. FIG. 7E shows antibody binding to coronavirus spike proteins expressed on the cell surface. GX-Pangolin, RaTG13, and GD-Pangolin are SARS-CoV-2 related viruses and the rest are non-human ACE2 dependent viruses.

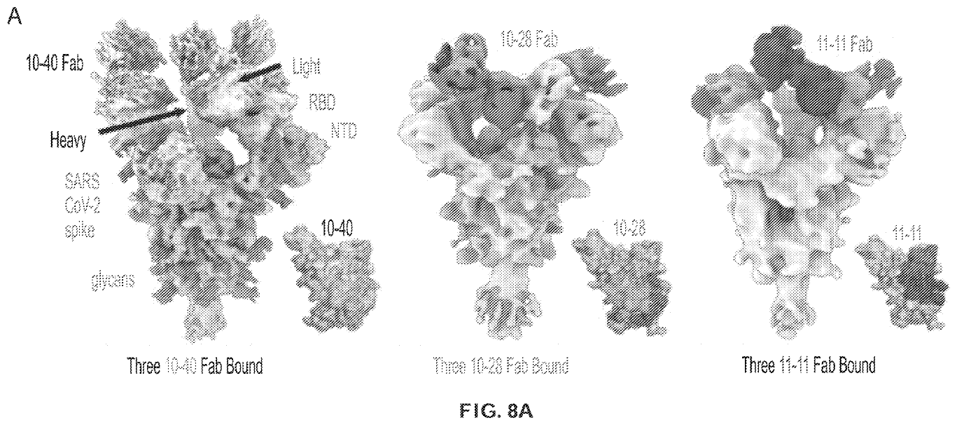

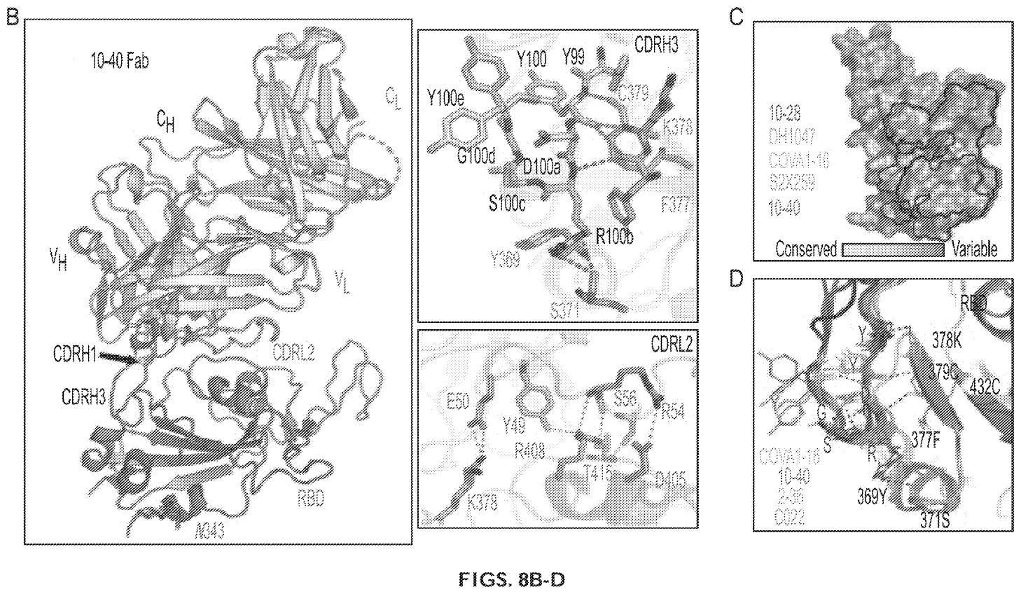

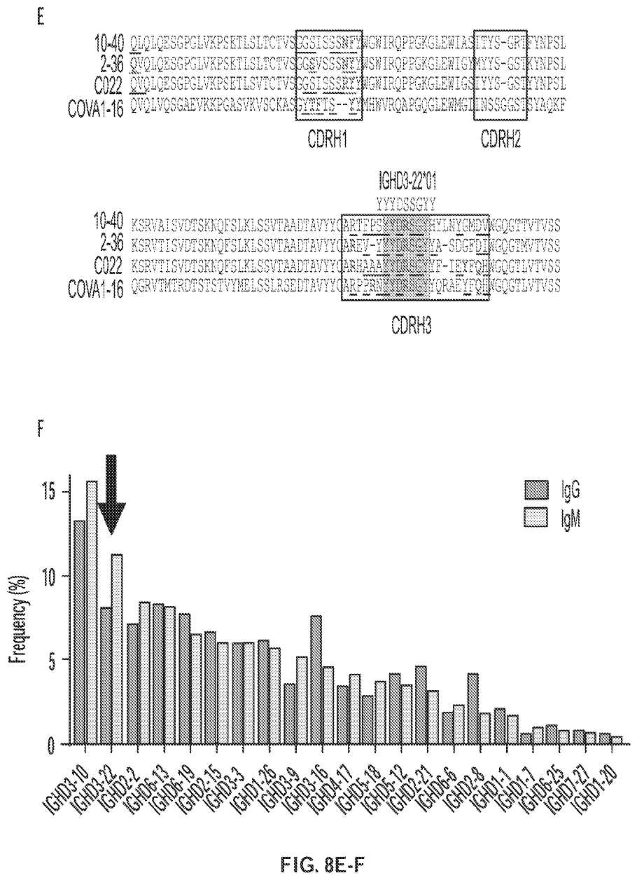

FIGS. 8A-F show structural and bioinformatic analyses of isolated antibodies. FIG. 8A shows Cryo-EM reconstructions and recognition footprints for monoclonal antibodies 10-40, 10-28, and 11-11 Fabs bound to the B.1.351 spike protein. The spike is colored in light gray, with the receptor binding domains (RBDs) in green with the glycans in purple, oriented with the membrane towards the bottom. The 10-40 Fabs are colored in red, the 10-28 Fabs in yellow, and the 11-11 Fabs in dark green. The Fab heavy chains are shaded darker, and the light chain shaded lighter. The footprint of each respective antibody on the inner face of RBD is displayed next to each spike. FIG. 8B shows crystal structure of the 10-40 Fab bound to USA-WA1/2020 SARS-COV-2 RBD. FIG. 8C shows a footprint comparison of 10-28, 10-40, DH1047, COVA-1-16, and S2X259 epitope footprints on SARS-COV-2 RBD. The RBD is colored according to the sequence conservation of each residue across 52 sarbecoviruses. FIG. 8D shows a comparison of 10-40, COVA1-16, and 2-36 CDRH3s produced by superimposing RBDs from each complex. The CDRH3s of the three antibodies bind to RBD with a highly similar mode. FIG. 8E shows heavy chain sequence alignment of antibodies 10-40, 2-36, C022, and COVA1-16, with the CDRH3 aligned with the germline sequence of the IGHD3-22*01 gene. The paratope residues are underlined and residues that form hydrogen bonds with RBD are colored in red. FIG. 8F shows D gene usage in antibody repertoires among healthy donors, ranked by frequency.

FIGS. 9A-E show prophylactic protection against a mouse adapted strain of SARSCOV-2 by engineered antibody 10-40. FIG. 9A shows that mutations in the MA10 virus do not overlap with the epitope of 10-40. The binding epitope of 10-40 is denoted in purple and the ACE2 binding epitope is denoted in blue. FIG. 9B shows neutralization of the mouse adapted strain of SARS-COV-2 (MA10) by antibody 10-40 in Vero E6 cells. FIG. 9C shows an experimental timeline of a protection study in SARS-COV-2 MA10 challenged mice. FIG. 9D shows body weight change in each group. Percent changes in body weight change of individual mice at day 3 shown (N=8 mice/group). Two-tailed t-test with Wilcoxon matched-pairing used to determine significance indicated by p values. FIG. 9E shows TCID50/gm of lung from 5-fold titration of lung homogenate of each mice.

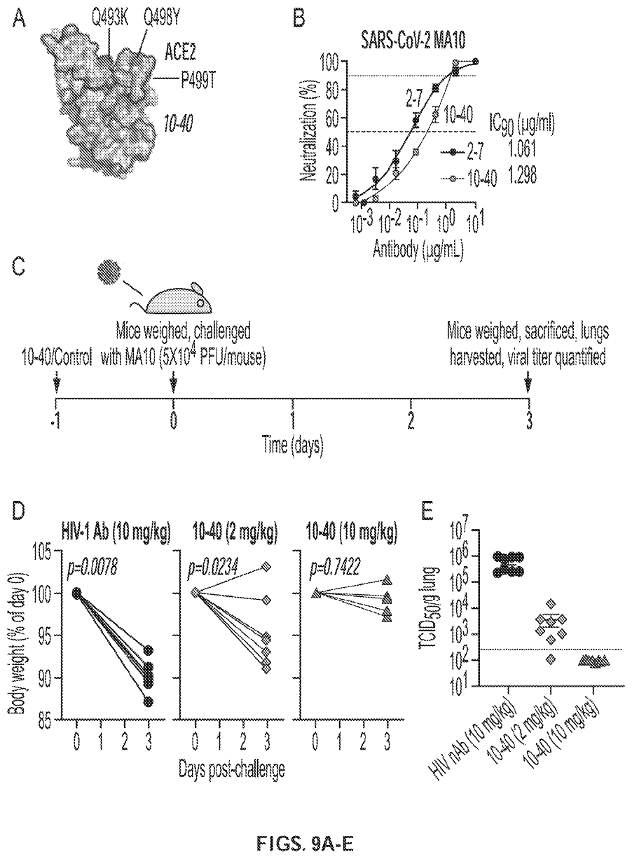

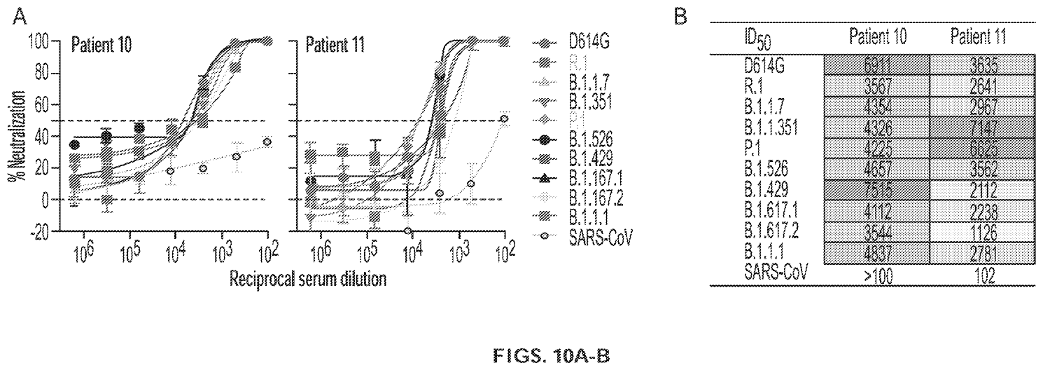

FIGS. 10A-B show neutralization profiles of the convalescent sera selected for antibody isolation (FIG. 10A) and a summary of ID₅₀ titers against each virus (FIG. 10B). Sera from two patients are tested for neutralization against VSV pseudotyped with the spike of SARS-COV-2 variants or SARS-COV spikes. Data are shown as mean±SD of three technical replicates.

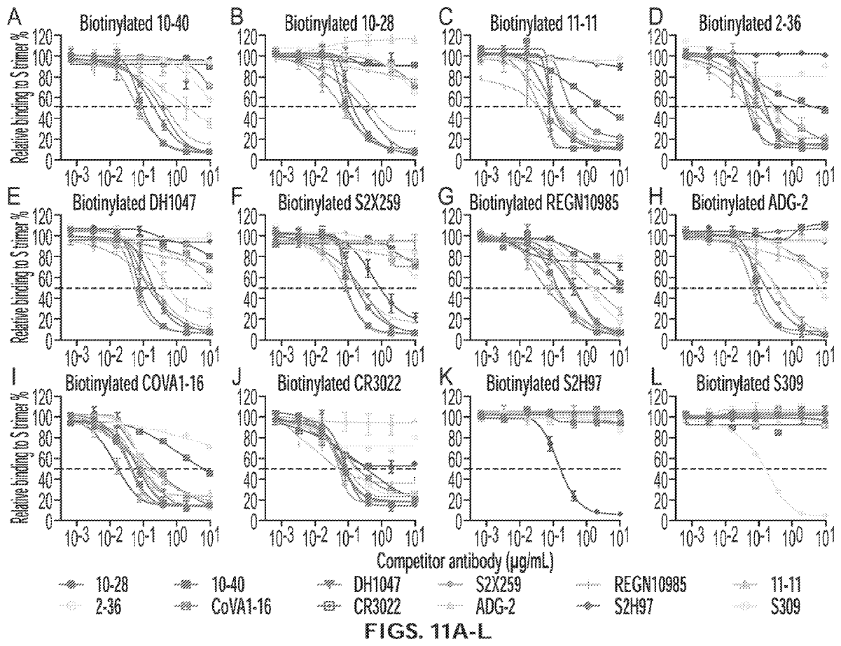

FIGS. 11A-L show individual binding curves for competition ELISA. Data are shown as mean±SD of two technical replicates. FIG. 11A shows individual binding curves to Spike trimer where the antibodies disclosed herein compete with biotinylated 10-40 antibody. FIG. 11B shows individual binding curves to Spike trimer where the antibodies disclosed herein compete with biotinylated 10-28 antibody. FIG. 11C shows individual binding curves to Spike trimer where the antibodies disclosed herein compete with biotinylated 11-11 antibody. FIG. 11D shows individual binding curves to Spike trimer where the antibodies disclosed herein compete with biotinylated 2-36 antibody. FIG. 11E shows individual binding curves to Spike trimer where the antibodies disclosed herein compete with biotinylated DH1047 antibody. FIG. 11F shows individual binding curves to Spike trimer where the antibodies disclosed herein compete with biotinylated S2X259 antibody. FIG. 11G shows individual binding curves to Spike trimer where the antibodies disclosed herein compete with biotinylated REGN10985 antibody. FIG. 11H shows individual binding curves to Spike trimer where the antibodies disclosed herein compete with biotinylated ADG-2 antibody. FIG. 11I shows individual binding curves to Spike trimer where the antibodies disclosed herein compete with biotinylated COVA1-16 antibody. FIG. 11J shows individual binding curves to Spike trimer where the antibodies disclosed herein compete with biotinylated CR3022 antibody. FIG. 11K shows individual binding curves to Spike trimer where the antibodies disclosed herein compete with biotinylated S2H97 antibody. FIG. 11L shows individual binding curves to Spike trimer where the antibodies disclosed herein compete with biotinylated S309 antibody.

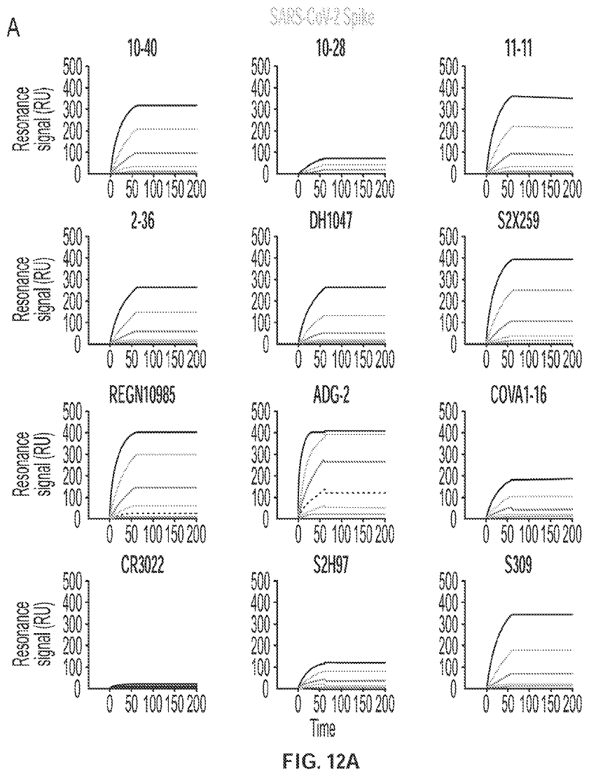

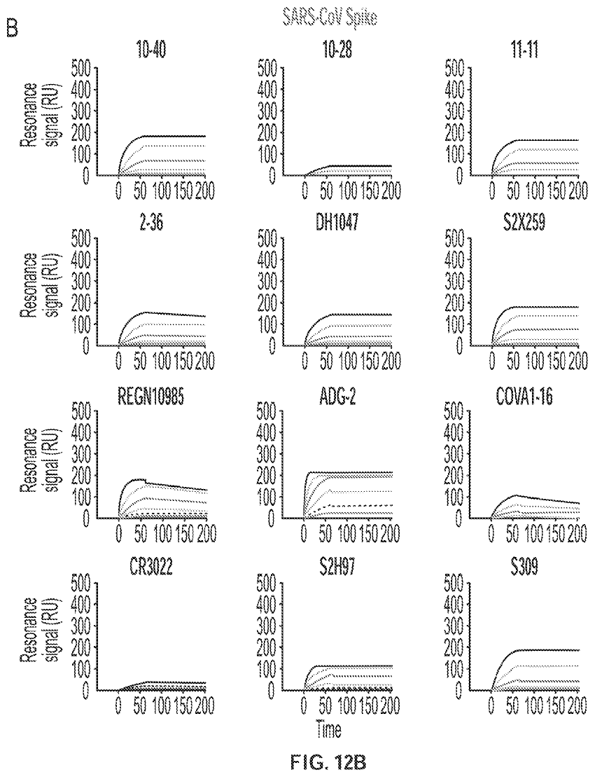

FIGS. 12A-C show binding kinetics and affinities of engineered monoclonal antibodies 10-28, 10-40, and 11-11, and other reported broad antibodies. Binding of antibodies to non-variant SARS-COV-2 (FIG. 12A) and SARS-COV (FIG. 12B) spike proteins were determined by surface plasmon resonance (SPR). The spike protein bound sensors were incubated with six different concentrations of antibodies. Kinetic data from one representative experiment were fit to a 1:1 binding model. (FIG. 12C) Summary of SPR kinetic and affinity measurements.

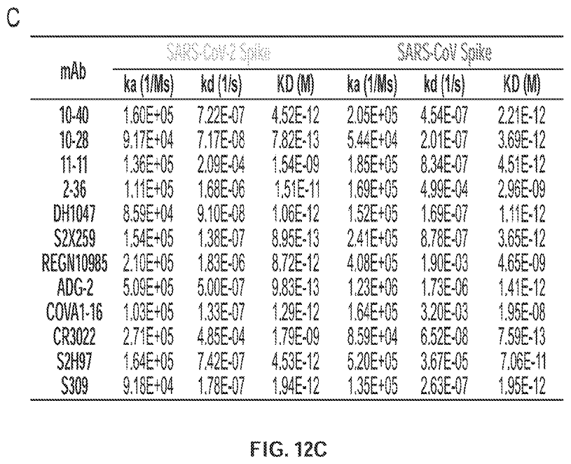

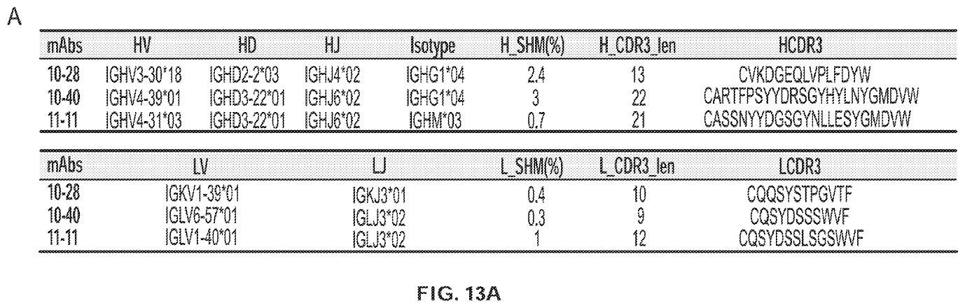

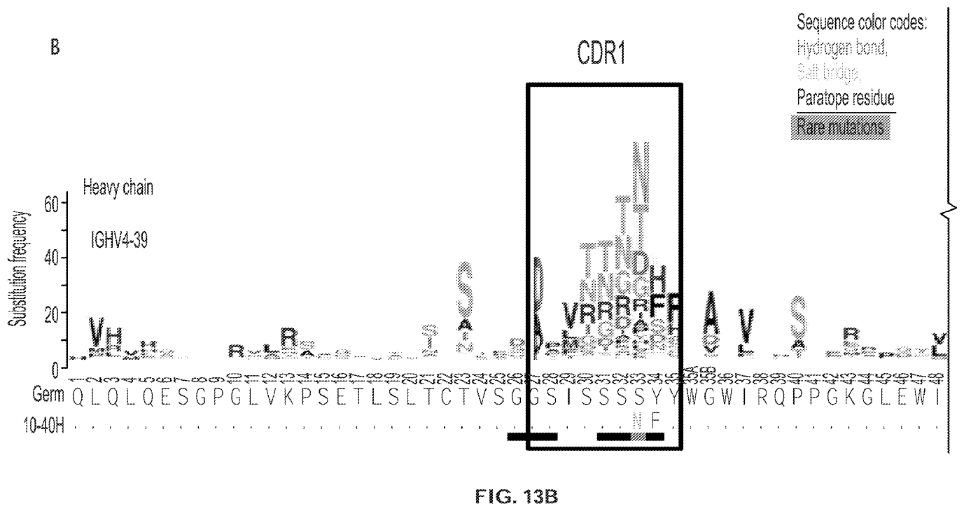

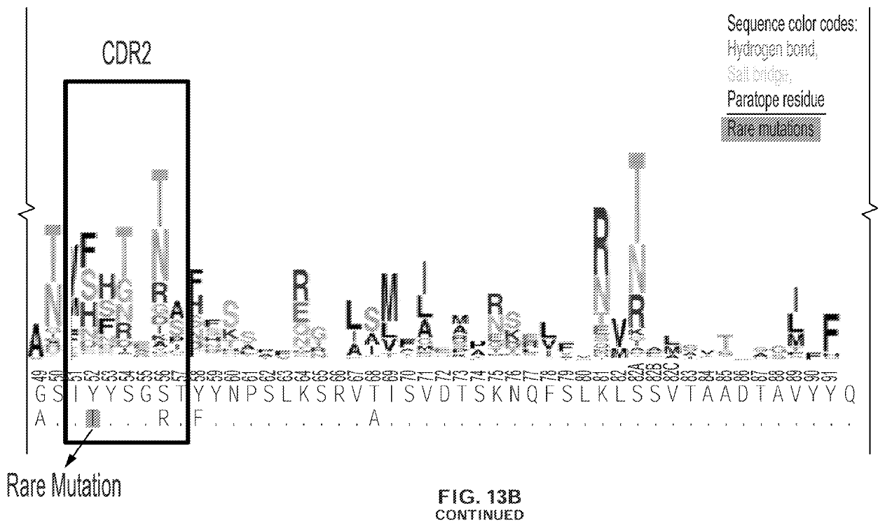

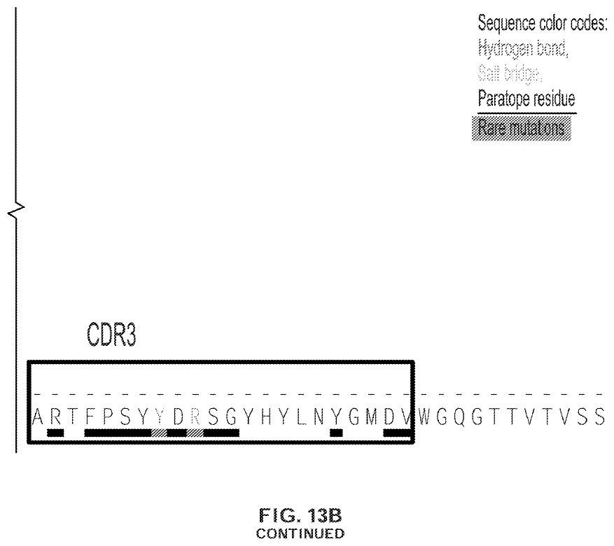

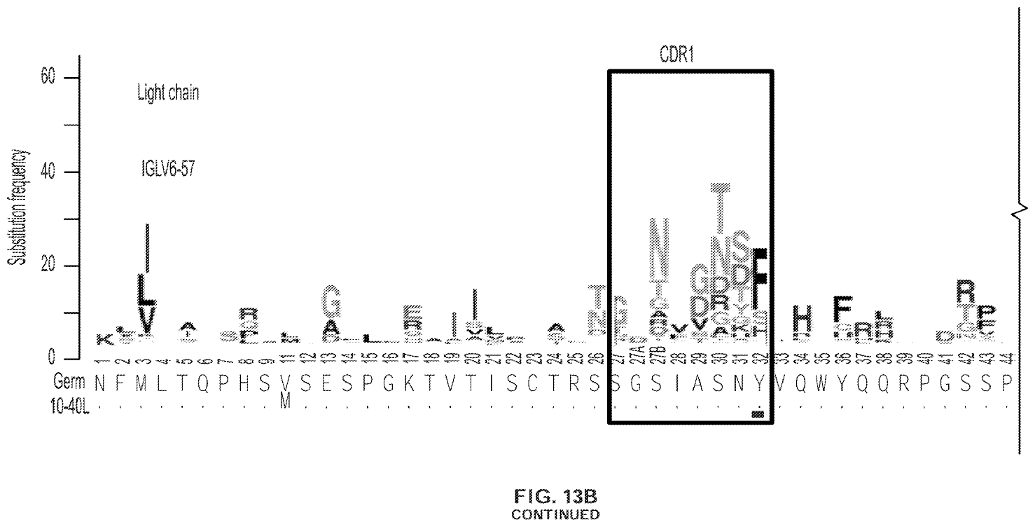

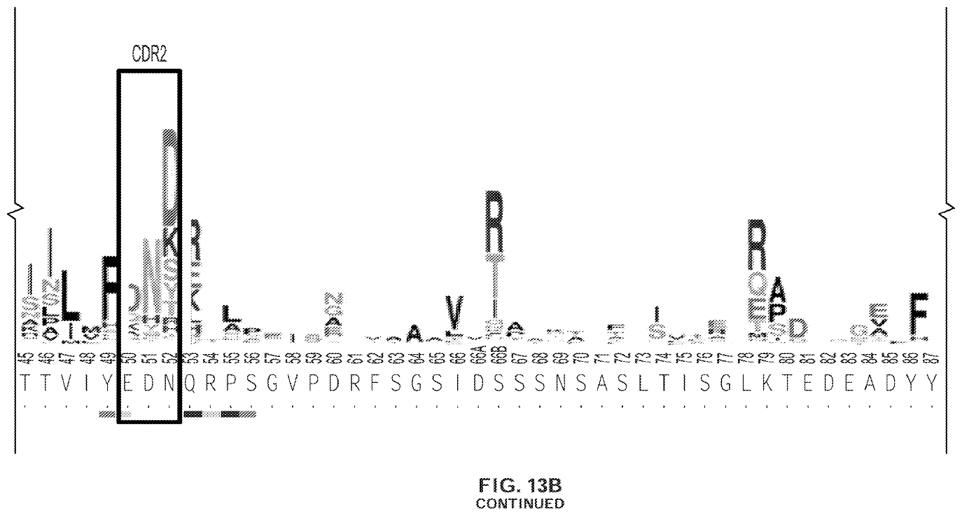

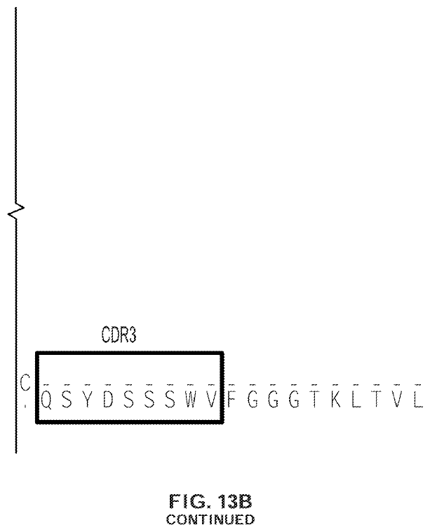

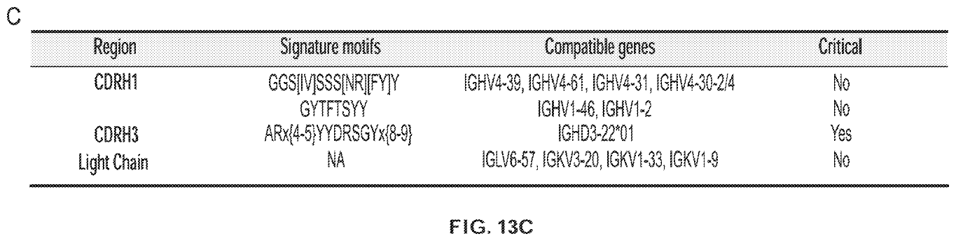

FIGS. 13A-C shows a genetic analysis of engineered monoclonal antibodies 10-40, 10-28, and 11-11. FIG. 13A shows genetic annotation for the three broadly neutralizing antibodies in this study, 10-28, 10-40, and 11-11. FIG. 13B shows a sequence alignment of antibody 10-40 with its germline genes, and gene specific substitution profile (GSSP) for IGHV4-39 and IGLV6-57. The dots denote conserved residues. The Y52T substitution in the 10-40 heavy chain is colored in red to indicate a rare mutation. FIG. 13C shows the signature of the 10-40-like reproducible antibody class.

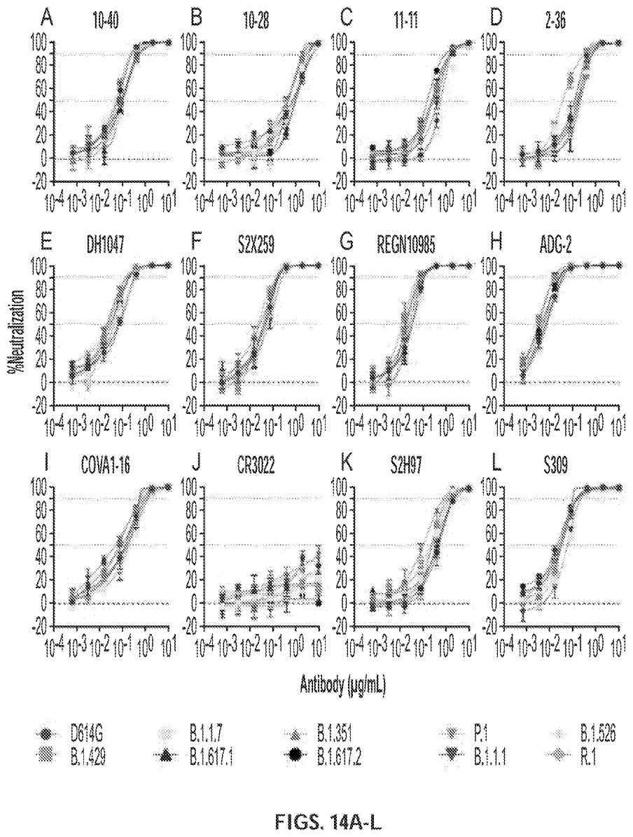

FIGS. 14A-L show the neutralization profiles of antibodies against SARS-COV-2 variants. Neutralization curves of antibodies against VSV pseudotypes with SARS-COV-2 spikes. FIG. 14A shows neutralization curves of engineered mAb 10-40 against SARS-COV-2 variants. FIG. 14B shows neutralization curves of engineered mAb 10-28 against SARS-CoV-2 variants. FIG. 14C shows neutralization curves of engineered mAb 11-11 against SARS-COV-2 variants. FIG. 14D shows neutralization curves of mAb 2-36 against SARS-CoV-2 variants. FIG. 14E shows neutralization curves of antibody DH1047 against SARS-CoV-2 variants. FIG. 14F shows neutralization curves of antibody S2X259 against SARS-CoV-2 variants. FIG. 14G shows neutralization curves of antibody REGN10985 against SARS-COV-2 variants. FIG. 14H shows neutralization curves of antibody ADG-2 against SARS-COV-2 variants. FIG. 14I shows neutralization curves of antibody COVA1-16 against SARS-COV-2 variants. FIG. 14J shows neutralization curves of antibody CR3022 against SARS-COV-2 variants. FIG. 14K shows neutralization curves of antibody S2H97 against SARS-COV-2 variants. FIG. 14L shows neutralization curves of antibody S309 against SARS-COV-2 variants. Data are shown as mean±SD of three technical replicates.

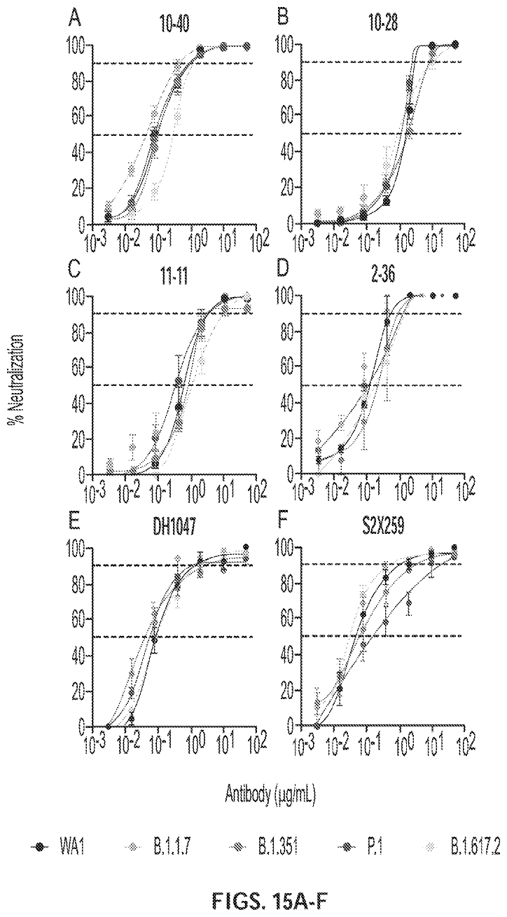

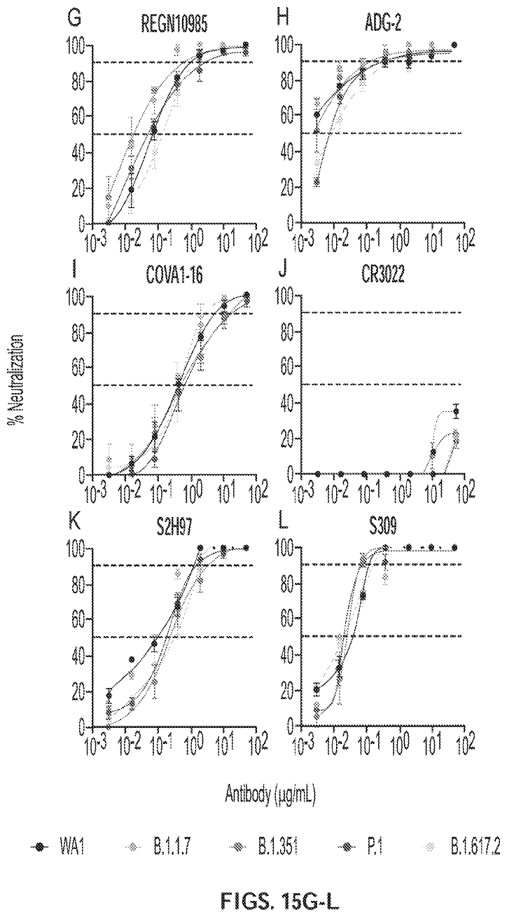

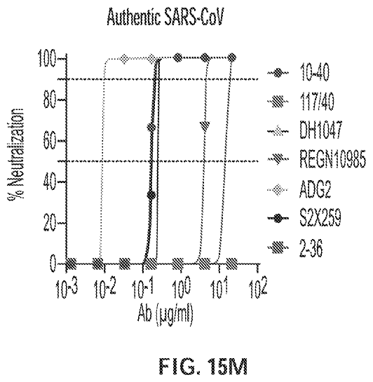

FIGS. 15A-M show neutralization profiles of antibodies against authentic SARS-CoV-2 variant viruses. Neutralization curves of antibodies against authentic SARS-COV-2 strains in a cytopathic effect reduction assay. Data are shown as mean±SEM of three technical replicates. FIG. 15A shows neutralization profiles of engineered mAb 10-40 against authentic SARS-COV-2 variant viruses. FIG. 15B shows neutralization profiles of engineered mAb 10-28 against authentic SARS-COV-2 variant viruses. FIG. 15C shows neutralization profiles of engineered mAb 11-11 against authentic SARS-COV-2 variant viruses. FIG. 15D shows neutralization profiles of mAb 2-36 against authentic SARS-COV-2 variant viruses. FIG. 15E shows neutralization profiles of antibody DH1047 against authentic SARS-COV-2 variant viruses. FIG. 15F shows neutralization profiles of antibody S2X259 against authentic SARS-COV-2 variant viruses. FIG. 15G shows neutralization profiles of antibody REGN10985 against authentic SARS-COV-2 variant viruses. FIG. 15H shows neutralization profiles of antibody ADG-2 against authentic SARS-COV-2 variant viruses. FIG. 15I shows neutralization profiles of antibody CAVA1-16 against authentic SARS-COV-2 variant viruses. FIG. 15J shows neutralization profiles of antibody CR3022 against authentic SARS-CoV-2 variant viruses. FIG. 15K shows neutralization profiles of antibody S2H97 against authentic SARS-COV-2 variant viruses. FIG. 15L shows neutralization profiles of antibody S309 against authentic SARS-COV-2 variant viruses. FIG. 15M shows neutralization profiles of antibodies 10-40, 117/40, DH1047, REGN10985, ADG2, S2X259, and 2-36 against authentic SARS-COV.

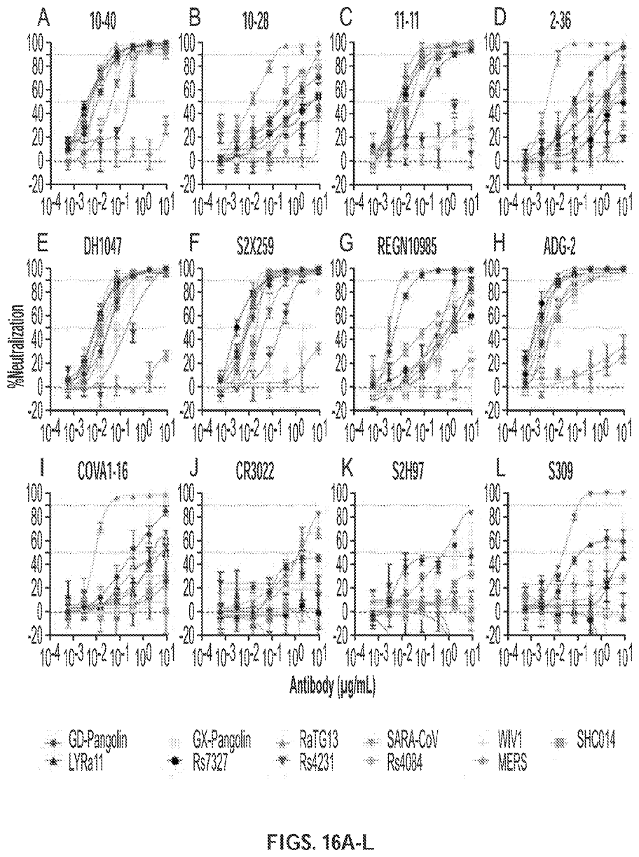

FIGS. 16A-L show neutralization profiles of antibodies against sarbecoviruses. Neutralization curves of antibodies against VSV pseudotypes with sarbecovirus spike proteins. FIG. 16A shows neutralization profiles of engineered mAb 10-40 against sarbecoviruses. FIG. 16B shows neutralization profiles of engineered mAb 10-28 against sarbecoviruses. FIG. 16C shows neutralization profiles of engineered mAb 11-11 against sarbecoviruses. FIG. 16D shows neutralization profiles of antibody 2-36 against sarbecoviruses. FIG. 16E shows neutralization profiles of antibody DH1047 against sarbecoviruses. FIG. 16F shows neutralization profiles of antibody S2X259 against sarbecoviruses. FIG. 16G shows neutralization profiles of antibody REGN10985 against sarbecoviruses. FIG. 16H shows neutralization profiles of antibody ADG-2 against sarbecoviruses. FIG. 16I shows neutralization profiles of antibody COVA1-16 against sarbecoviruses. FIG. 16J shows neutralization profiles of antibody CR3022 against sarbecoviruses. FIG. 16K shows neutralization profiles of antibody S2H97 against sarbecoviruses. FIG. 16L shows neutralization profiles of antibody S309 against sarbecoviruses. Data are shown as mean±SD of three technical replicates.

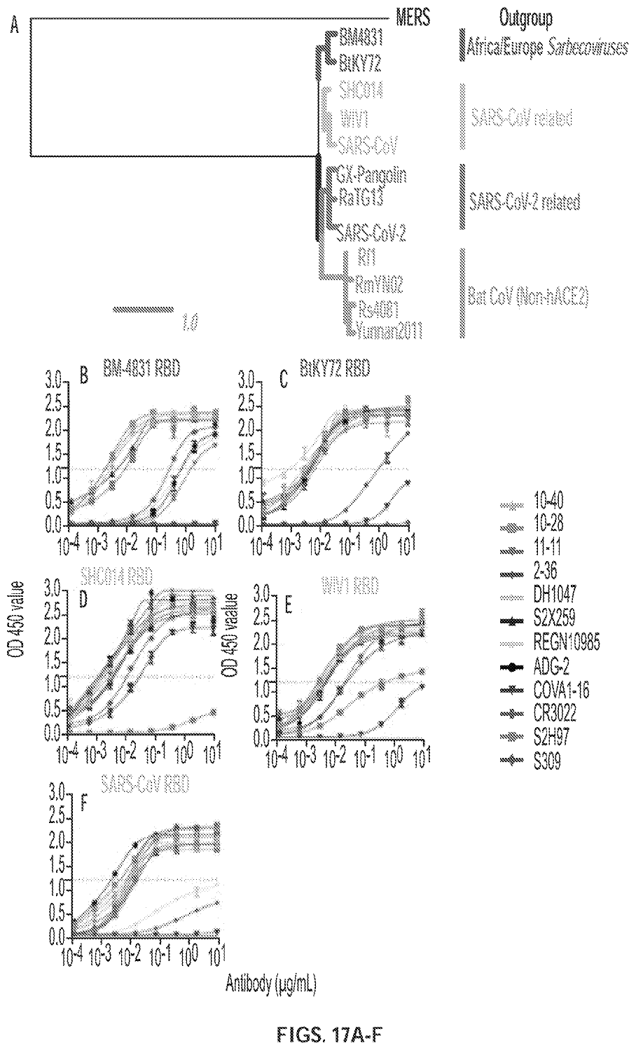

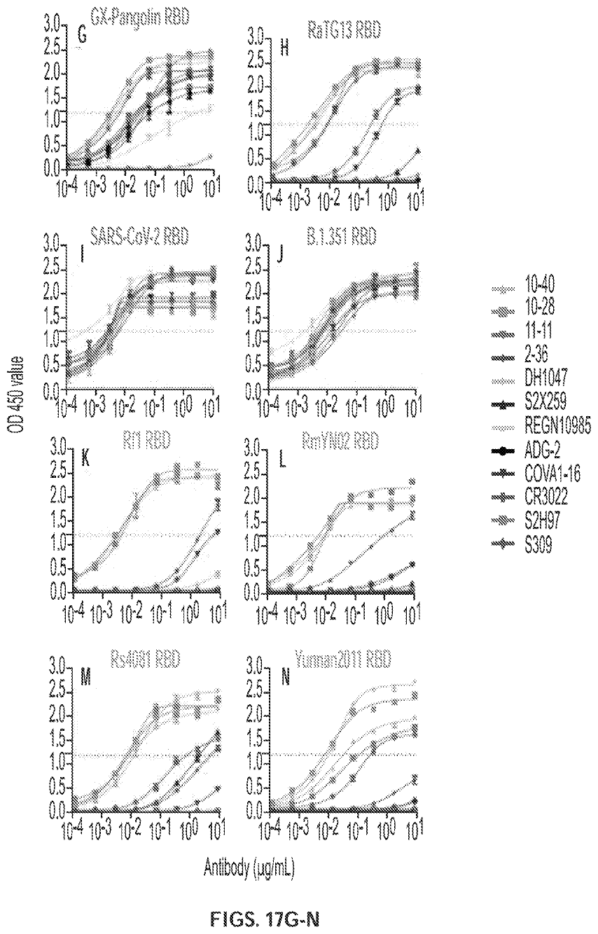

FIGS. 17A-N show breadth of cross-reactive antibodies examined by RBD-based ELISA. FIG. 17A shows a phylogenetic tree of coronaviruses based on RBD protein sequences. Four phylogenetically distinct sarbecovirus groups are indicated in different colors. FIGS. 17B-N show binding of the selected antibodies disclosed herein to the panel of RBD proteins as tested by ELISA. FIG. 17B shows antibody binding to the BM-4831 RBD. FIG. 17C shows antibody binding to the BtKY72 RBD. FIG. 17D shows antibody binding to the SHC014 RBD. FIG. 17E shows antibody binding to the WIV1 RBD. FIG. 17F shows antibody binding to the SARS-COV RBD. FIG. 17G shows antibody binding to the GX-Pangolin RBD. FIG. 17H shows antibody binding to the RaTG13 RBD. FIG. 17I shows antibody binding to the SARS-COV-2 RBD. FIG. 17J shows antibody binding to the B.1.351 RBD. FIG. 17K shows antibody binding to the Rf1 RBD. FIG. 17L shows antibody binding to the RmYN02 RBD. FIG. 17M shows antibody binding to the Rs4081 RBD. FIG. 17N shows antibody binding to the Yunnan2011 RBD.

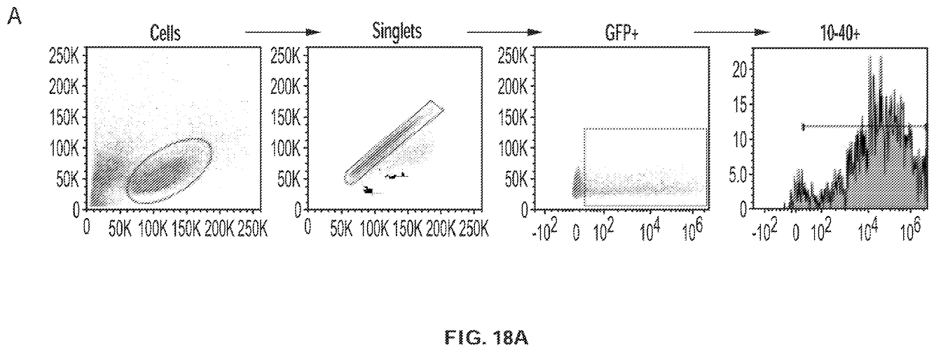

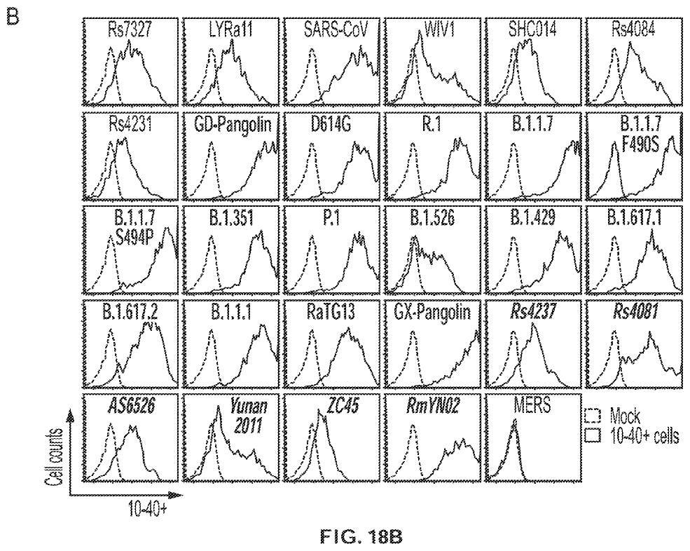

FIGS. 18A-B show binding of engineered monoclonal antibody 10-40 to coronavirus spike-expressing cells. FIG. 18A shows gating strategy for quantification of 10-40-positive cells from Expi293 cells transiently transfected to express coronavirus spike proteins. FIG. 18B shows the binding profiles of engineered monoclonal antibody 10-40 to spike trimers expressed on Expi293 cell surface.

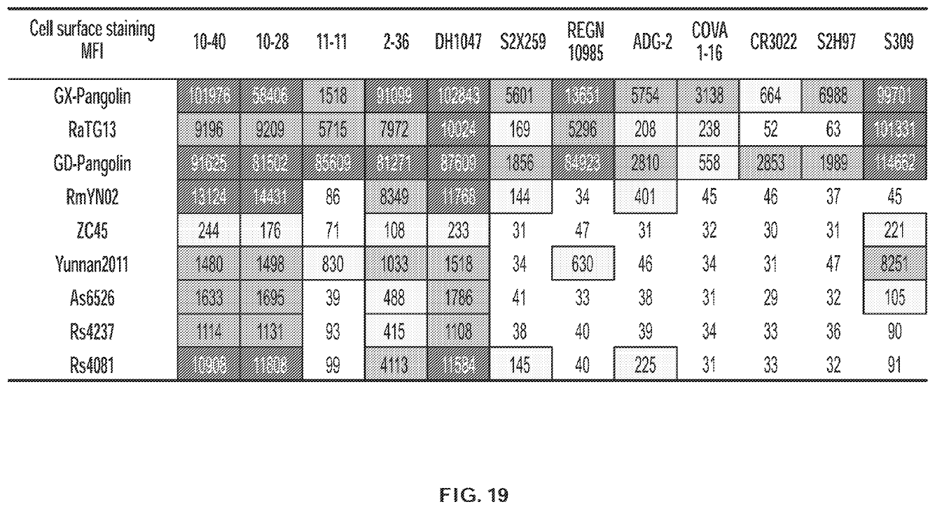

FIG. 19 shows mean fluorescent intensity (MFI) of antibody bound to spike-expressing cells. The data shown are representative data from one of three independent experiments.

FIGS. 20A-E show Cryo-EM data processing for engineered monoclonal antibody 10-40 in complex with SARS-COV-2 B.1.351 spike trimer. FIG. 20A shows representative micrograph, power spectrum, and contrast transfer function (CTF) fit. FIG. 20B shows representative 2D class averages showing spike particles. FIG. 20C shows global consensus refinement Fourier Shell Correlation (FSC) curve (top panel) and particle projection viewing angle distribution (bottom panel). FIG. 20D shows local resolution estimation mapped on surface density for global refinement. FIG. 20E shows crystal structure model for 10-40 Fab rigid body docked into cryo-EM map, viewed from the outside (left) and from the inside (right).

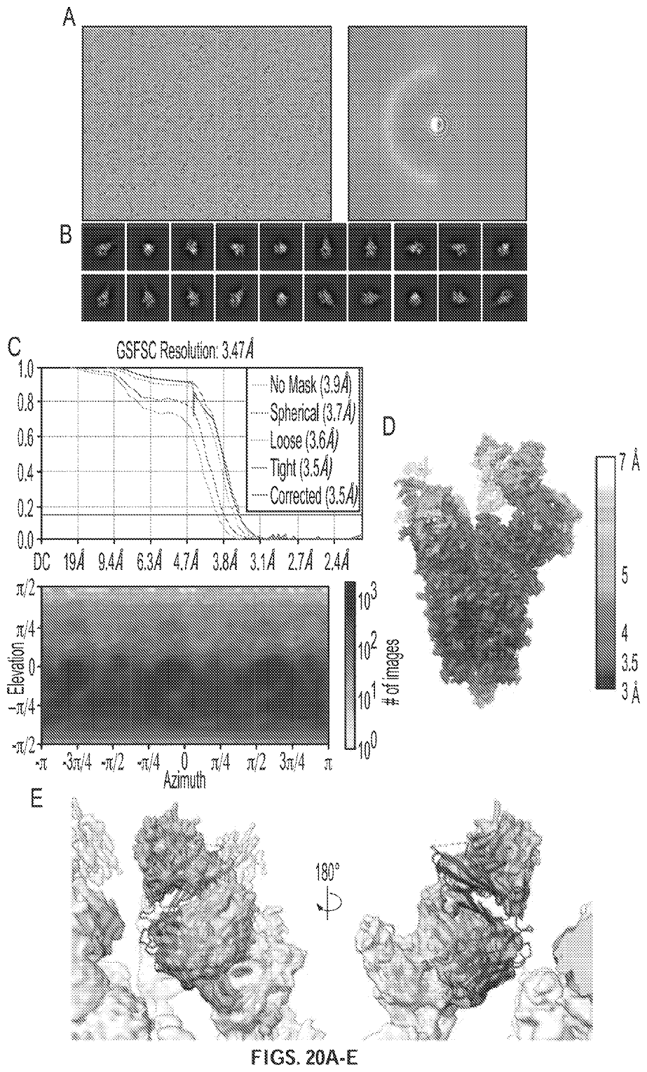

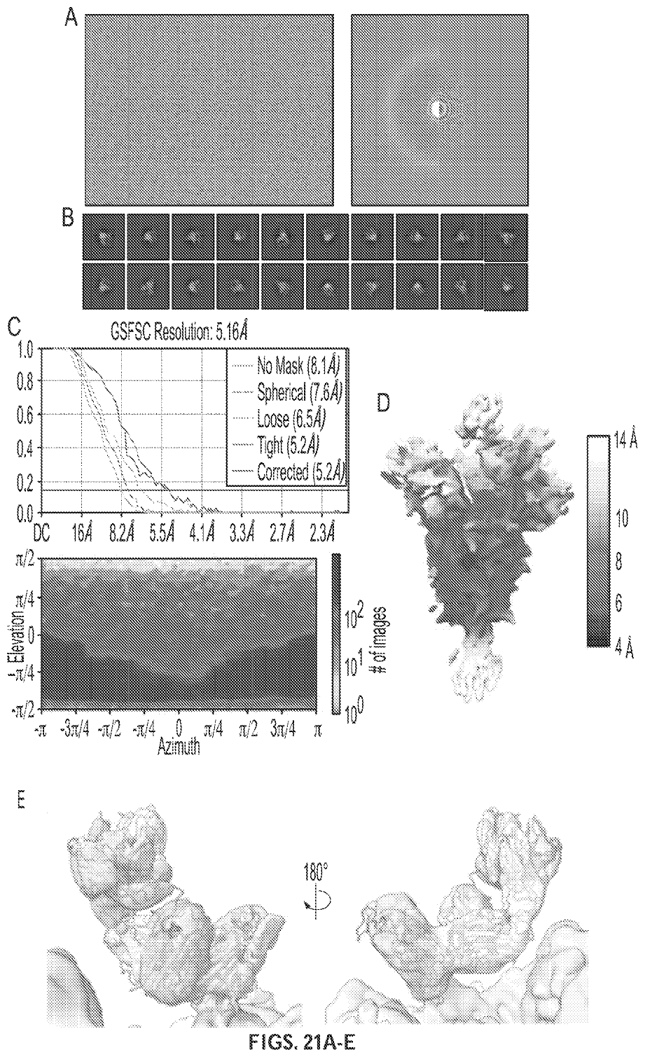

FIGS. 21A-E show Cryo-EM data processing for engineered monoclonal antibody 10-28 in complex with SARS-COV-2 B.1.351 spike trimer. FIG. 21A shows representative micrograph, power spectrum, and contrast transfer function (CTF) fit. FIG. 21B shows representative 2D class averages showing spike particles. FIG. 21C shows a global consensus refinement Fourier Shell Correlation (FSC) curve (top panel) and particle projection viewing angle distribution (bottom panel). FIG. 21D shows local resolution estimation mapped on surface density for global refinement. FIG. 21E shows a crystal structure model for 10-28 Fab rigid body docked into cryo-EM map, viewed from the outside (left) and from the inside (right).

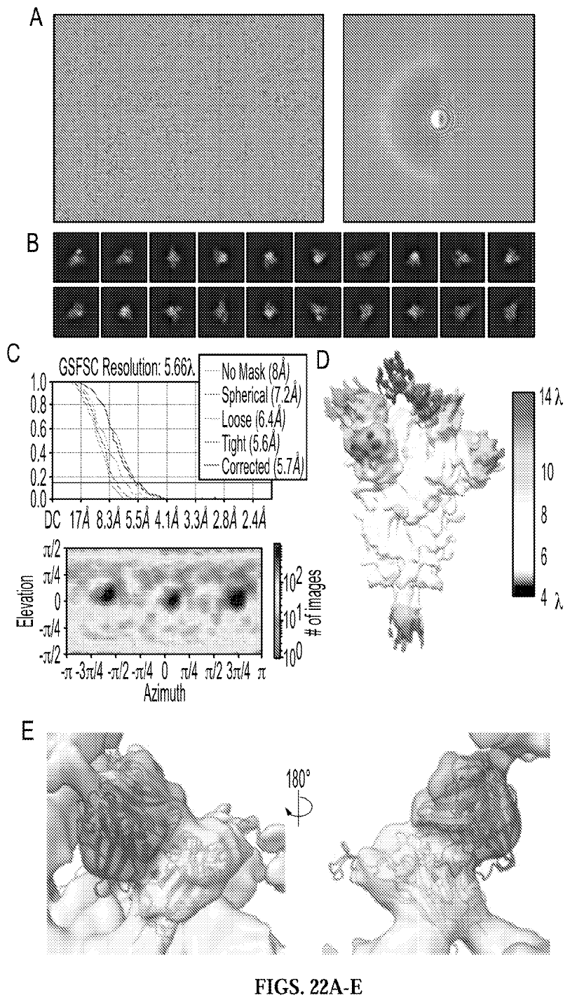

FIGS. 22A-E show Cryo-EM data processing for engineered monoclonal antibody 11-11 in complex with SARS-COV-2 B.1.351 spike trimer. FIG. 22A shows representative micrograph, power spectrum, and contrast transfer function (CTF) fit. FIG. 22B shows representative 2D class averages showing spike particles. FIG. 22C shows global consensus refinement Fourier Shell Correlation (FSC) curve (top panel) and particle projection viewing angle distribution (bottom panel). FIG. 22D shows local resolution estimation mapped on surface density for global refinement. FIG. 22E shows homology model for 11-11 Fab rigid body docked into cryo-EM map, viewed from the outside (left) and from the inside (right).

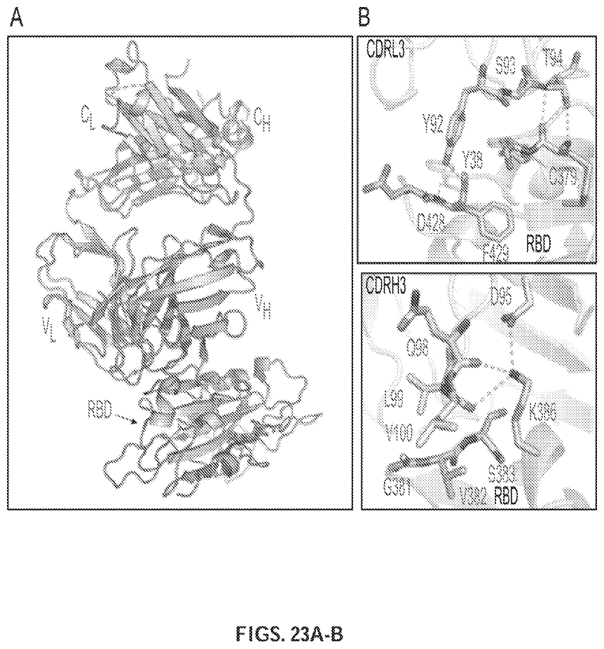

FIGS. 23A-B show crystal structures. FIG. 23A shows crystal structure of engineered monoclonal antibody 10-28 complexed with non-variant SARS-COV-2 RBD. RBD is colored in green, heavy and light chains are in dark and light yellow. FIG. 23B shows expanded view of 10-28 interactions with RBD recognition by CDRL3 (upper panel), and recognition by CDRH3 (lower panel), colored as in (A).

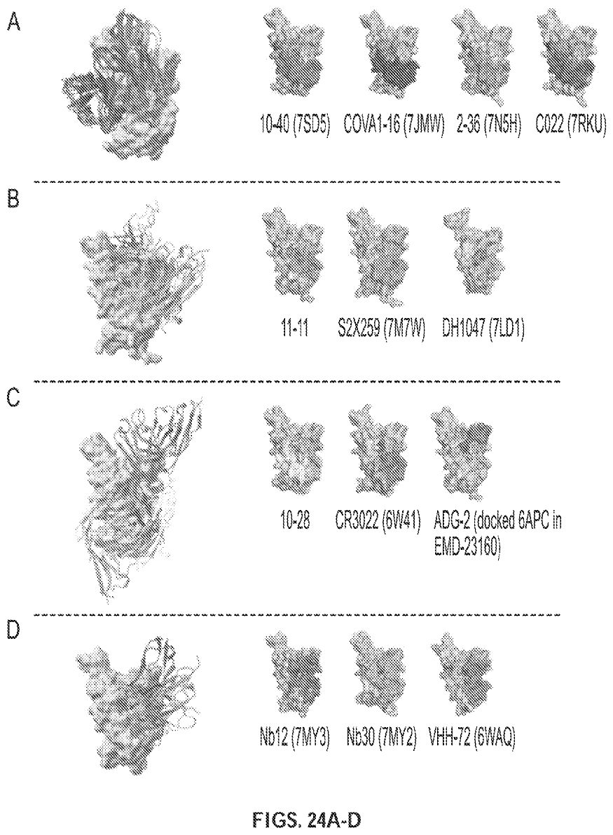

FIGS. 24A-D show footprint comparison between engineered monoclonal antibodies 10-40, 11-11, and 10-28, and published antibodies. FIG. 24A shows that antibodies 10-40, COVA1-16, and 2-36 recognize the inner side of the inner face of RBD with a similar angle of approach. FIG. 24B shows that antibodies 11-11, S2X259, and DH1047 recognize the outer side of the inner face of RBD with a similar angle of approach. FIG. 24C shows that antibodies 10-28 and CR3022 bind to the lower middle side of the inner face of RBD. ADG-2 binds to the upper side of the inner face of RBD. Atomic coordinates are not available for ADG-2, thus, it is modeled by docking a homology model based on 6APC into the map EMD-23160, as described by (ADG-2 citation). FIG. 24D shows nanobodies that broadly neutralize also recognize the inner face of RBD from the outer side, similar to antibodies in panel B.

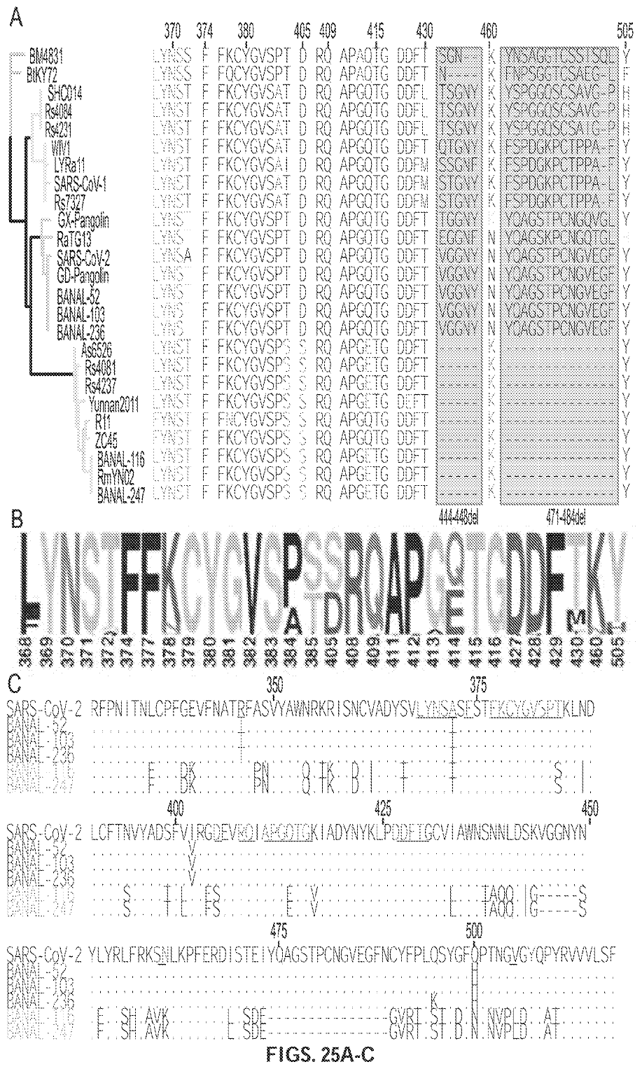

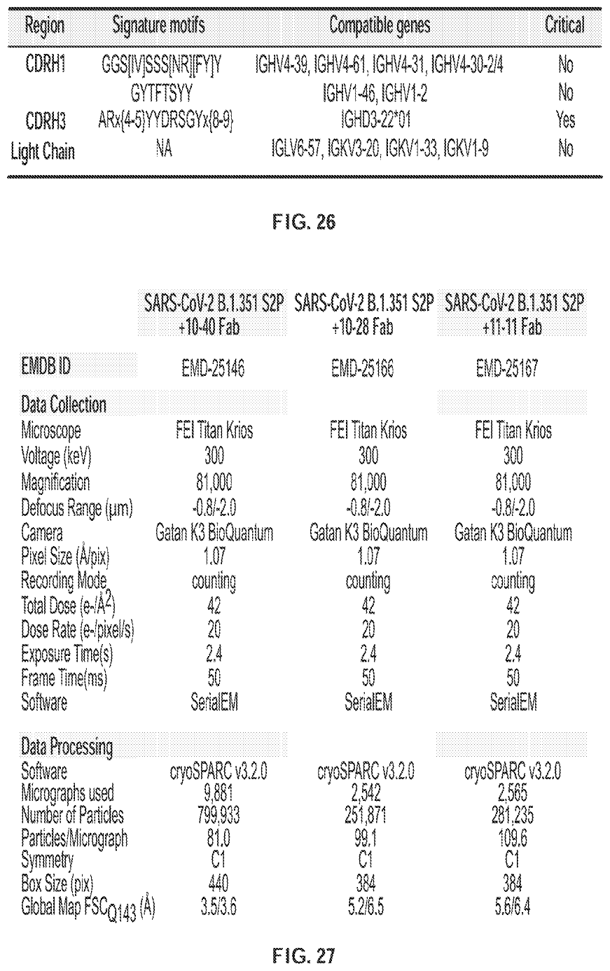

FIGS. 25A-C show that engineered monoclonal antibody 10-40 recognizes a conserved region within RBD. FIG. 25A shows a sequence alignment of sarbecoviruses within the epitope of 10-40. FIG. 25B shows sequence conservation of antibody 10-40 epitope in sarbecoviruses. FIG. 26C shows epitope residues for antibody 10-40 (red colored), compare with recently identified 5 related viruses. Blue represents human-ACE2 dependent viruses, yellow are non-ACE2 dependent viruses.

FIG. 26 shows the signature of 10-40-like reproducible antibody class.

FIG. 27 shows Cryo-EM data collection and processing.

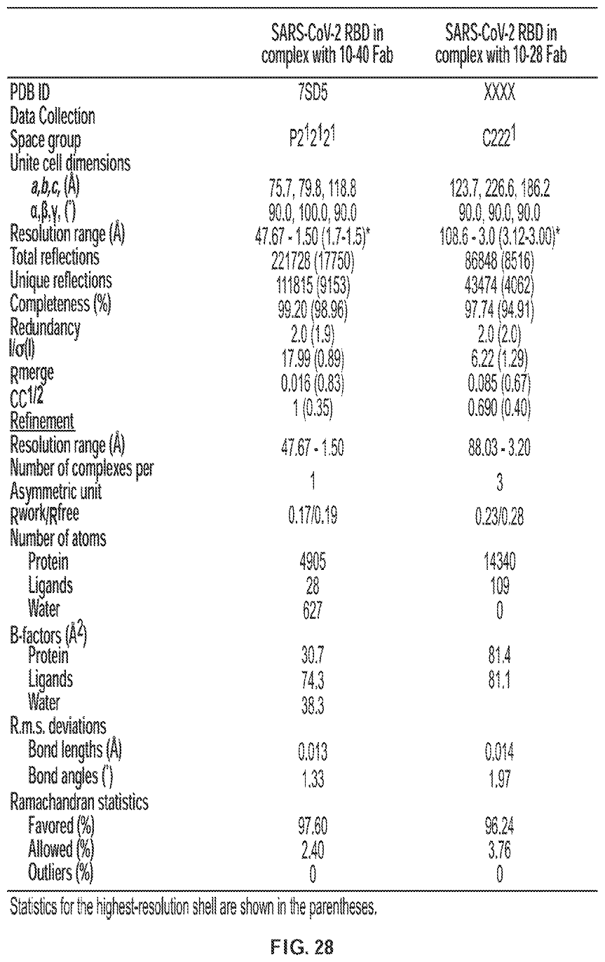

FIG. 28 shows X-ray diffraction data collection and refinement statistics.

### Example 3-Systems and Method for Engineering Monoclonal Antibodies Against SARS-CoV-2 and its Variants

The engineered monoclonal antibodies 12-16 and 12-19 described herein exhibit broad neutralization capability against SARS-COV-2 variants of concern and variants of interest. Engineered monoclonal antibodies 12-16 and 12-19 exhibit also neutralizing activity against Omicron variants. These engineered antibodies were isolated from serum of patients with a SAR-COV-2 infection. Structural analyses described herein has highlighted that the 12-16 and 12-19 engineered monoclonal antibodies both recognize the SARS-COV-2 spike protein at a novel and conserved quaternary epitope. This epitope is at the juncture between SD1 and NTD domains of the spike protein. Engineered monoclonal antibodies 12-16 and 12-19 are positioned in-between RBD/SD1 and NTD of the spike protein such that they form a ‘wedge’ that prevents the RBD of the spike protein from reaching the “up” position. As described herein, there are three different mechanisms of SARS-COV-2 neutralization by these NTD-targeting antibodies.

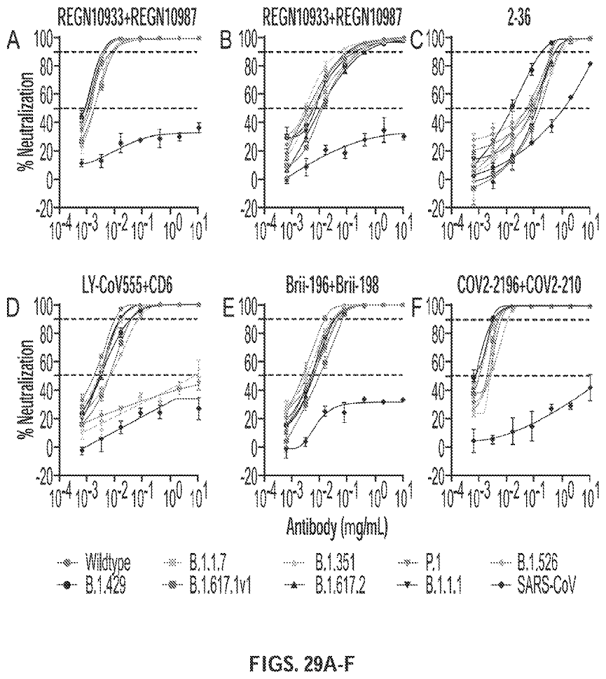

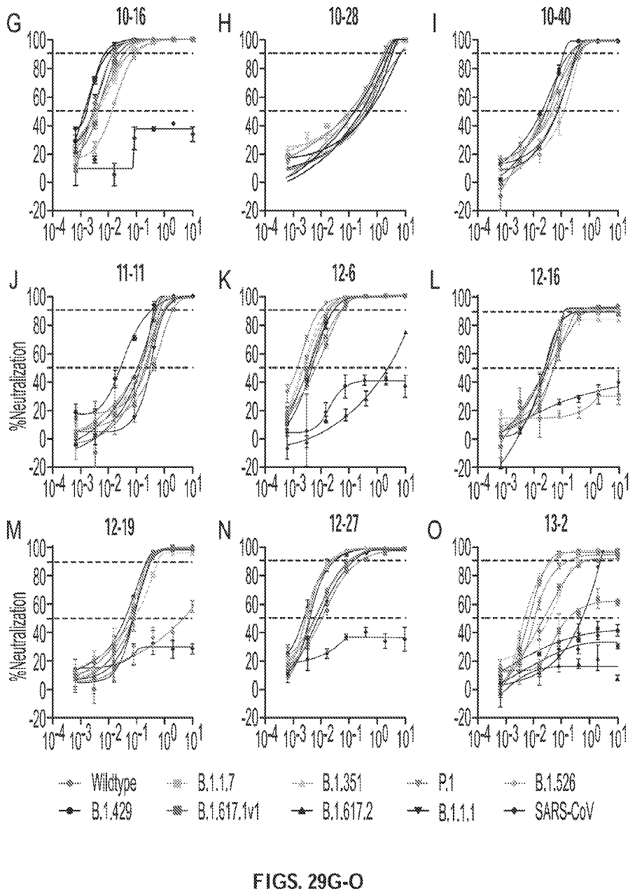

Neutralization profiles of down-selected monoclonal antibodies (mAbs) on wild-type (WT) SARS-COV-2 virus and pseudoviruses of SARS-COV-2 variants of concern (VOC PVs) are shows in FIGS. 29A-O. FIG. 29A shows neutralization with mAbs REGN10933 and REGN10987. FIG. 29B shows neuralization with mAb 2-7. FIG. 29C shows neutralization with mAb 2-36. FIG. 29D shows neutralization with mAbs LY-COV555 and CB6. FIG. 29E shows neutralization with mAbs Brii-196 and Brii-198. FIG. 29F shows neutralization with mAbs COV2-2196 and COV2-2130. FIG. 29G shows neutralization with engineered mAb 10-16. FIG. 29H shows neutralization with engineered mAb 10-28. FIG. 29I shows neutralization with engineered mAb 10-40. FIG. 29J shows neutralization with engineered mAb 11-11. FIG. 29K shows neutralization with engineered mAb 12-6. FIG. 29L shows neutralization with engineered mAb 12-16. FIG. 29M shows neutralization with engineered mAb 12-19. FIG. 29N shows neutralization with engineered mAb 12-27. FIG. 29O shows neutralization with engineered mAb 13-2. Monoclonal neutralizing antibodies 12-16 and 12-19 exhibit broad activity against wild-type SARS-COV-2 virus and its variants including B.1.1.7, B.1.351, B.1.526, B.1.429, B.1.617.1v1, B.1.617.2, and B.1.1.1.

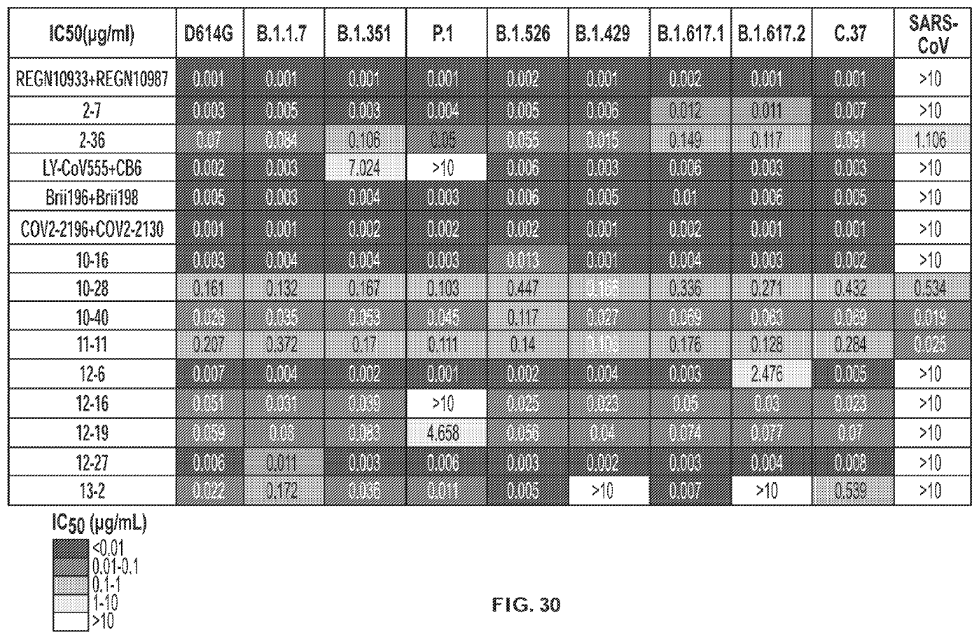

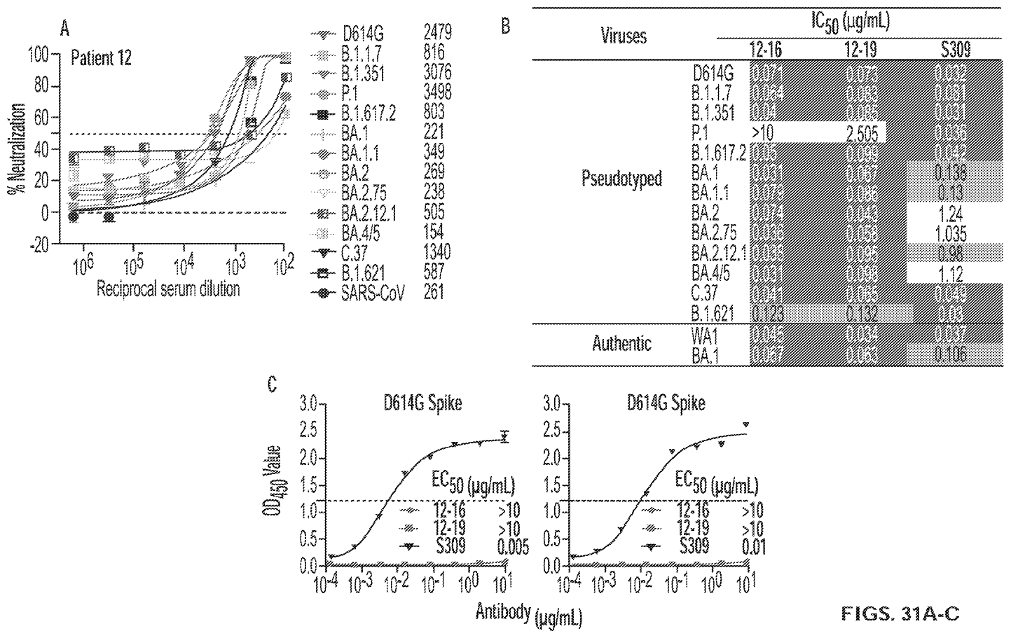

FIG. 30 shows the IC₅₀ values from neutralization of SARS-COV-2 virus and SARS-COV pseudoviruses with down-selected engineered monoclonal antibodies. Engineered monoclonal antibodies 12-16 and 12-19 exhibit IC₅₀ in the range of between 0.01 μg/mL and 0.1 μg/mL against SARS-COV-2 D614G, B.1.1.7, B.1.351, B.1.526, B.1.429, B.1.617.1, B.1.617.2, and C.37.

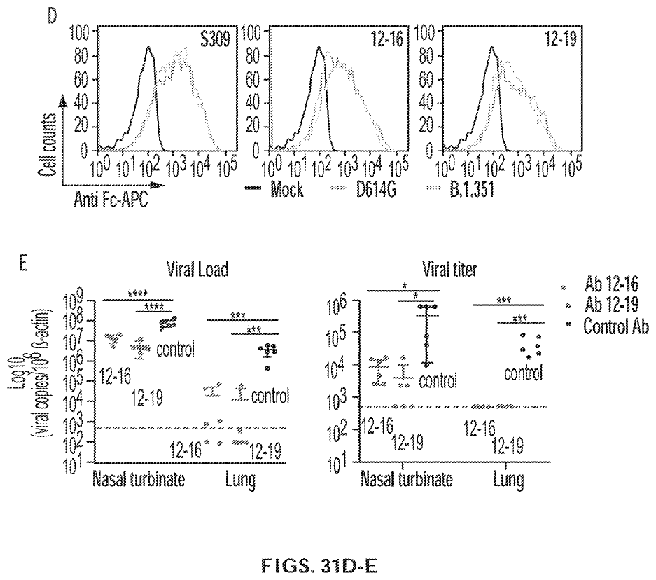

The in vitro and in vivo potency and breadth of neutralizing antibodies 12-16 and 12-19 are shows in FIGS. 31A-E. FIG. 31A shows neutralization titers (ID₅₀) of the convalescent serum from patient 12, which is selected for antibody isolation. VSV pseudotypes with the spike protein of SARS-COV-2 variants or the spike protein of SARS-CoV are neutralized. Data are shown as mean±SD of three technical replicates. FIG. 31B shows neutralization titers (IC₅₀) of selected engineered mAbs 12-16 and 12-19 against VSV pseudotypes with the spike protein of SARS-COV-2 or authentic viruses. Data are shown as mean±SD of three technical replicates. FIG. 31C shows that engineered antibodies 12-16 and 12-19 were tested by ELISA for binding to the spike protein trimers of the SARS-COV-2 D614G and B.1.351 viruses. Data are shown as mean±SD of two technical replicates. There is no binding observed for both antibodies. Antibody S309 is a positive control antibody, which potently neutralizes SARS-COV-2 and SARS-COV pseudoviruses as well as the authentic SARS-COV-2 virus, by engaging the receptor-binding domain (RBD) of the spike(S) glycoprotein FIG. 31D shows antibody binding to the SARS-COV-2 D614G (yellow) and B.1.351 (blue) spike proteins expressed on the cell surface. The data shown are representative data from one of three independent experiments. FIG. 31E shows prophylactic efficacy of selected mAbs in hamsters infected with Omicron variant. Three groups of hamsters (n=6 per group) received 10 mg/kg of the indicated mAb treatment by intraperitoneal injection one day before intranasal inoculation with 105 PFU of Omicron variant. Viral load (log 10 (RNA copies/106 β-actin copies)) and viral titers (log 10 (PFU per g)) were measured in trachea, lung of hamsters challenged with Omicron virus at 4 d.p.i. Error bars indicate mean±SD. Each symbol represents an individual hamster with a line indicating the mean of each group. P values were determined by one-way ANOVA followed by Tukey's multiple comparisons test. **P<0.01; ***P<0.001; ****P<0.0001. Dotted lines indicate assay limits of detection.

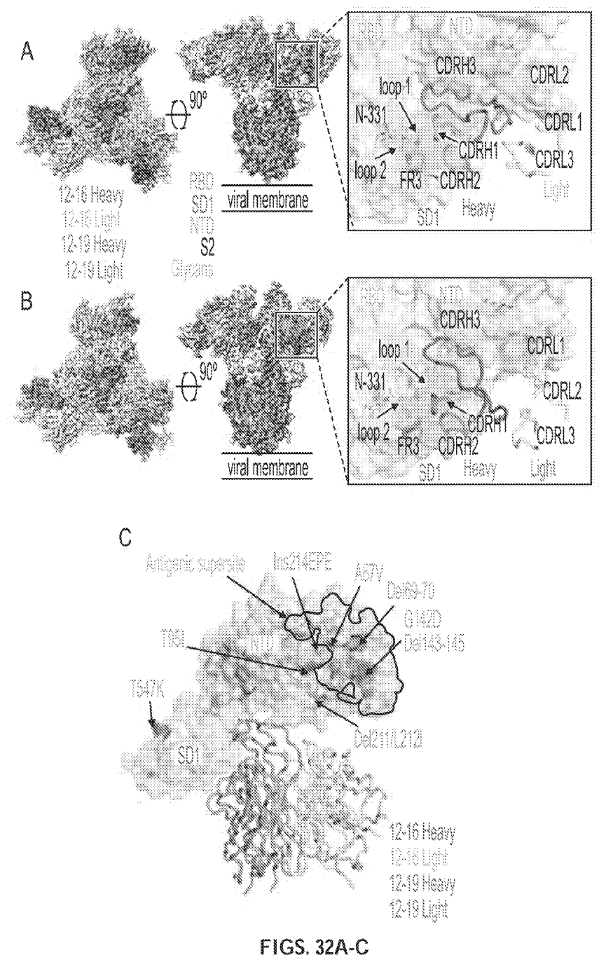

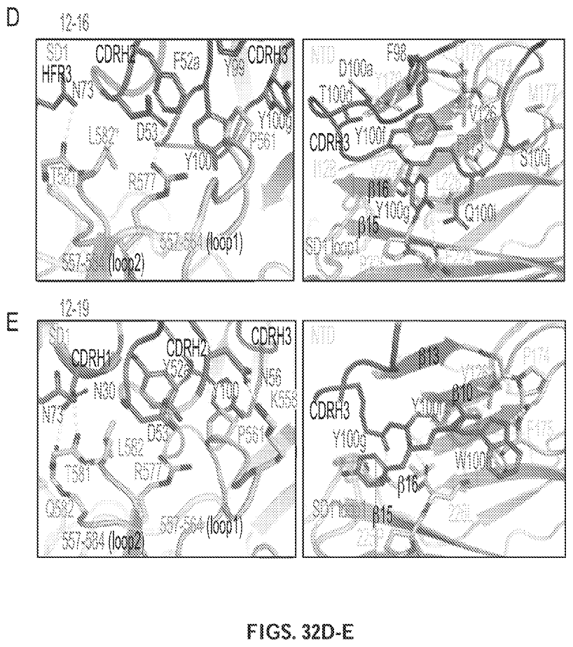

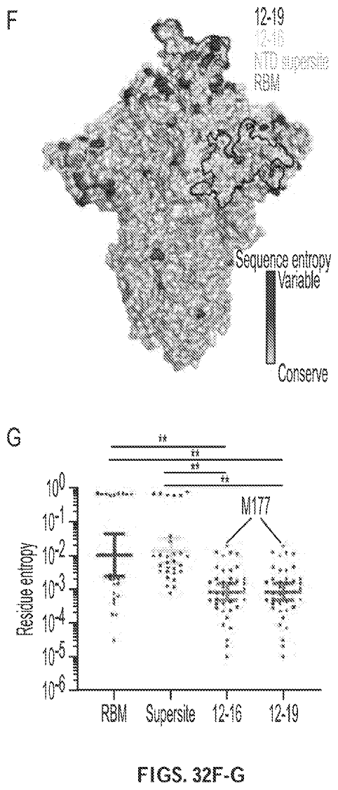

Engineered monoclonal antibodies 12-16 and 12-19 target a quaternary and conserved epitope between subdomain 1 (SD1) and the N-Terminal Domain (NTD) of a coronavirus spike protein as shown in FIGS. 32A-G. FIG. 32A shows CryoEM structure of engineered monoclonal antibody 12-16 in contact with a spike protein. The panels show a top view, a side view and the location of the complementarity-determining region (CDRs) of 12-16. The orange and green surface represent the footprint of 12-16 on NTD and SDI domain of a spike protein, respectively. FIG. 32B shows CryoEM structures of engineered monoclonal antibody 12-19 in contact with a spike protein. The panels show top view, side view and the location of the CDRs of 12-19. The orange and green surface represent the footprint of 12-19 on NTD and SD1, respectively. FIG. 32C shows antibodies 12-16 and 12-19 in complex with the NTD and SDI of a spike protein. The red sphere represent the mutations in Omicron variant of the spike protein, the NTD antigenic supersite is outlined in cyan. FIG. 32D shows interface residues and interaction for antibody 12-16 with SDI (left panel) and NTD (right panel). The hydrogen bonds are colored in yellow dashed lines. FIG. 32E shows interface residues and interaction for antibody 2-19 with SD1 (left panel) and NTD (right panel). The hydrogen bonds are colored in yellow dashed lines. FIG. 32F shows the footprint and residue entropy for the receptor-binding motif (RBM), NTD supersite, 12-16 and 12-19 epitope on the spike surface. FIG. 32G shows comparison of residue entropy between RBM, NTD supersite, 12-16 and 12-19 epitopes. Residues are represented as dots, and the geometric mean and 95% confidence interval (CI) of the 4 groups of residues are shown in blue, cyan, green and purple, respectively. The P values between different groups were calculated using the Kolmogorov-Smirnov test. The double asterisk symbol represents P<0.0001.

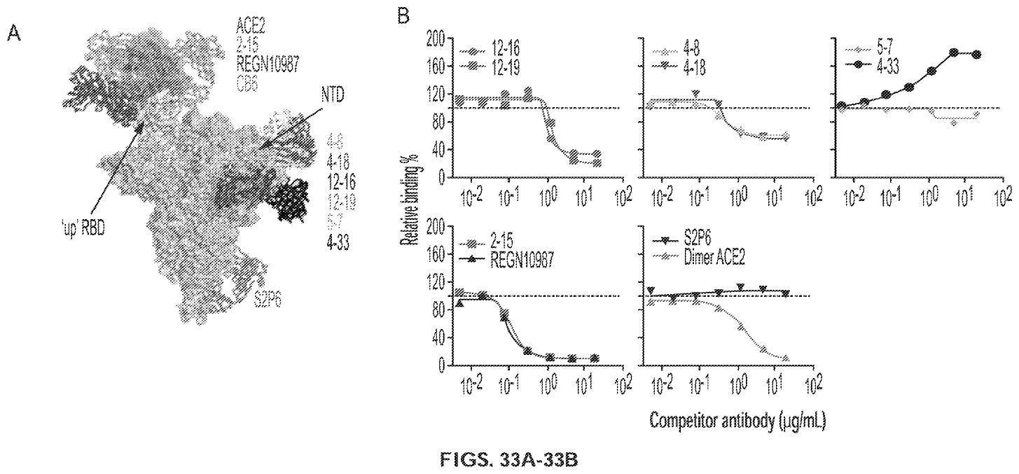

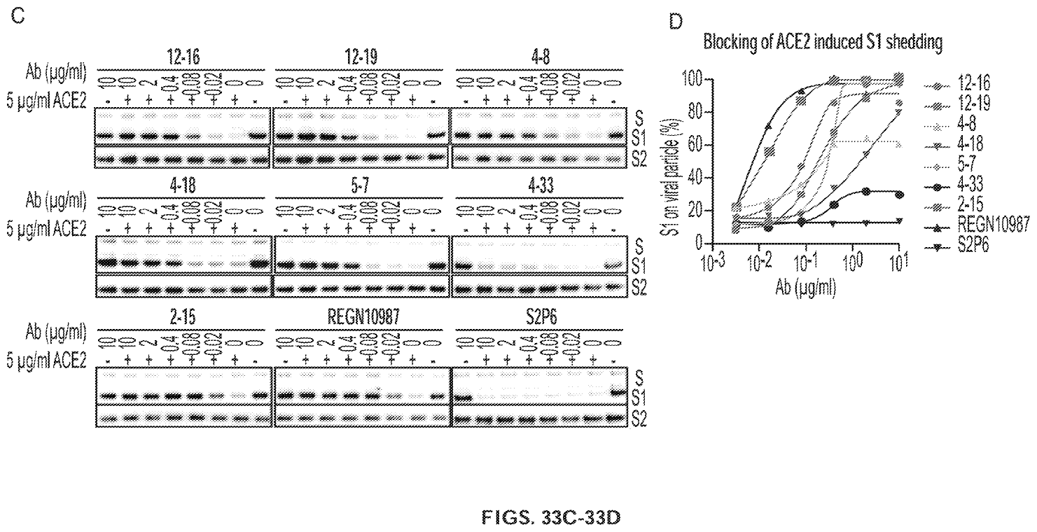

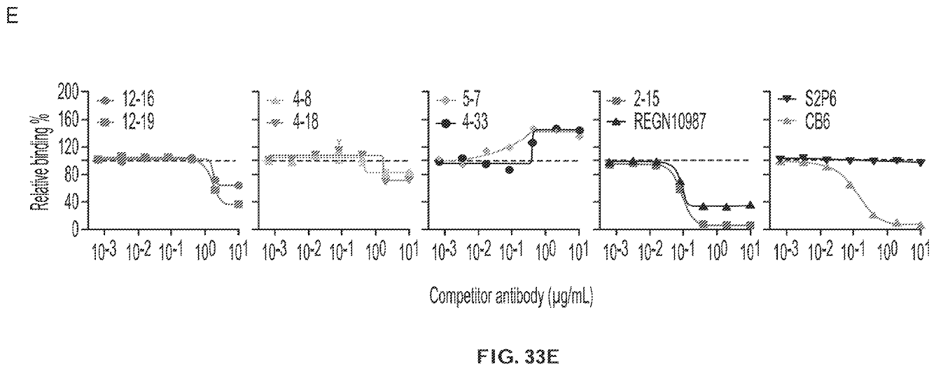

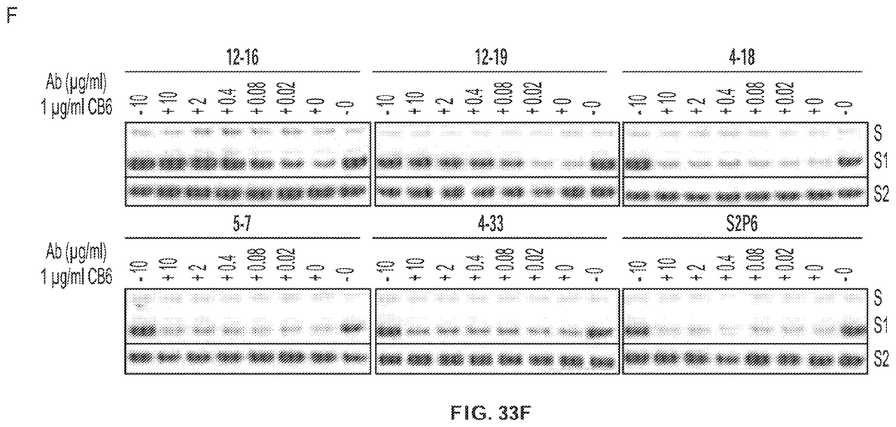

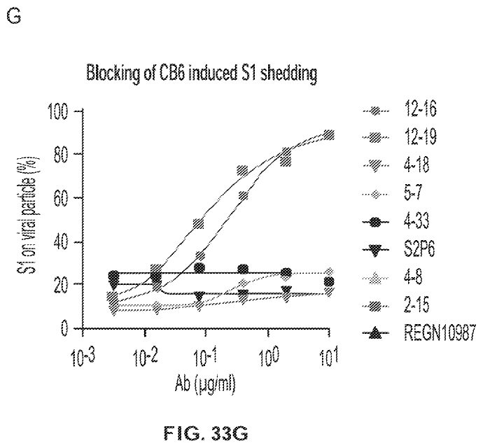

FIG. 33A-G show the mechanism of NTD-specific neutralizing mAbs against SARS-COV-2. FIG. 33A shows 10 selected antibodies in complex with hACE2 and 1 receptor binding domain (RBD) spike protein in the “up” conformation. 9 of them are neutralizing antibody except antibody 4-33. FIG. 33B shows competition of selected antibodies with ACE2 competing to bind to the SARS-COV-2 spike trimer expressed on Expi293 cell surface. Binding of ACE2 to the spike trimer was quantified by incubation of the cells in a solution of biotinylated dimeric ACE2, followed by staining the cells with Streptavidin-APC, washing the cells, and measuring fluorescent populations by flow cytometry. The anti-RBD 2-15 and REGN mAb were included as positive controls. The anti-S2 S2P6 antibody was used as a negative control. The data shown are representative data from one of three independent experiments. FIG. 33C shows blockage of soluble ACE2-induced shedding of SI from the S trimer on SARS-COV-2 virions by monoclonal antibodies. VSV particles pseudotyped with the variant SARS-COV-2 spike glycoproteins were pretreated with different doses of selected antibodies for half hour at 37° C., then incubated with the indicated concentration of soluble ACE2 (sACE2) for half hour at 37° C. Pelleted VSV particles were analyzed by Western blotting with antibodies against S1, S2. The data shown are representative data from one of three independent experiments. Engineered monoclonal antibodies 12-16 and 12-19 exhibit blockage of SI shedding in the concentration rage from 0 μg/mL to 0.08 μg/mL. FIG. 33D shows that the intensities of the SI and S2 glycoprotein bands in (C) were measured and the S1/S2 ratios are shown. FIG. 33E shows competition of selected antibodies with antibody CB6 for binding to a SARS-COV-2 spike trimer expressed on Expi293 cell surface. Binding of CB6 to the spike trimer was quantified by incubation of the cells in a solution of biotinylated dimeric CB6, followed by staining the cells with APC anti-human IgG Fc antibody, washing the cells, and measuring fluorescent populations by flow cytometry. The CB6 was included as a positive control. The anti-S2 S2P6 was used as a negative control. The data shown are representative data from one of three independent experiments. FIG. 33F shows blockage of soluble CB6-induced shedding of SI from the S trimer on SARS-COV-2 virions by indicated antibodies. VSV particles pseudotyped with the variant SARS-COV-2 S glycoproteins were pretreated with different doses of selected antibodies for half hour at 37° C., then incubated with the indicated concentration of CB6 for half hour at 37° C. Pelleted VSV particles were analyzed by Western blotting with antibodies against S1, S2. The data shown are representative data from one of three independent experiments. FIG. 33G shows that the intensities of the SI and S2 glycoprotein bands in (F) were measured and the S1/S2 ratios are shown.

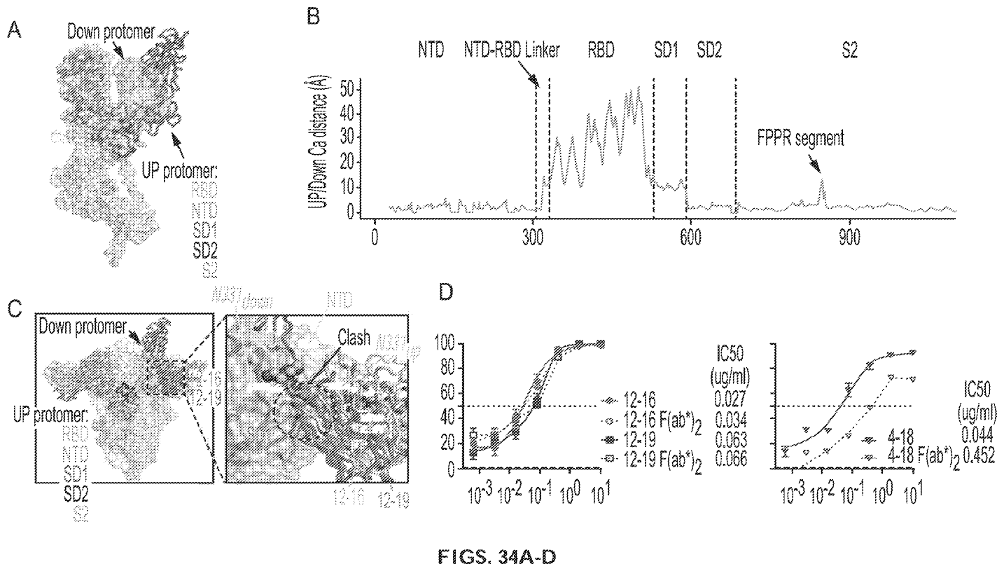

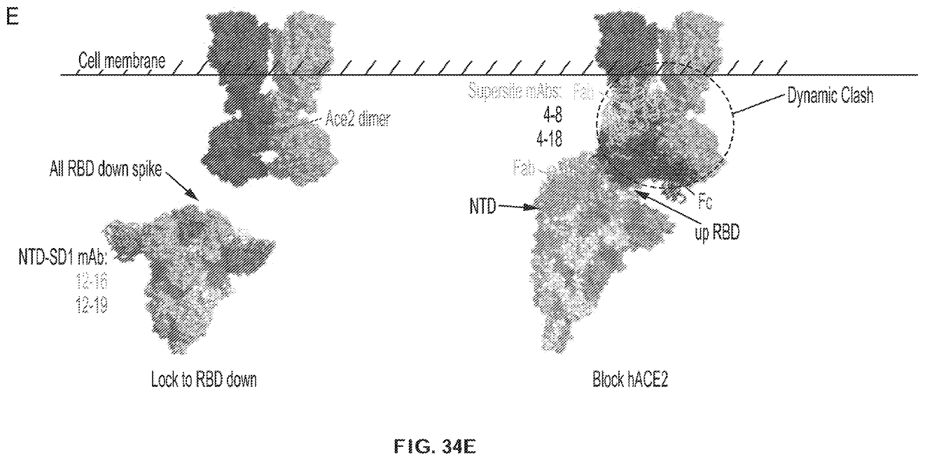

FIGS. 34A-E show structure-based neutralization mechanisms of engineered monoclonal antibodies 12-16, 12-19 and NTD. FIG. 34A shows superpose of RBD up and down protomers. The surface shows RBD down protomer and the ribbon shows RBD up protomer (PDB: 7krr). FIG. 34B shows per residue Ca distance of RBD up and down protomer showed in A. The dashed lines showed the boundary of each domain. FIG. 34C shows structural explanation of 12-16 and 12-19 block with RBD up protomer. Right panel shows the details of clashes occurred in SDIRDB-up with 12-16 and 12-19, the green and cyan sphere represent the N331 glycan in RBD-down and RBD-up protomer, respectively. FIG. 34D shows neutralization activities of monoclonal antibodies 12-16, 12-19 and 4-18 compared with their F(ab′) 2 forms. FIG. 34E shows structure models for spike protein bound with neutralizing antibodies (12-16, 12-19 and NTD antigenic site antibodies).

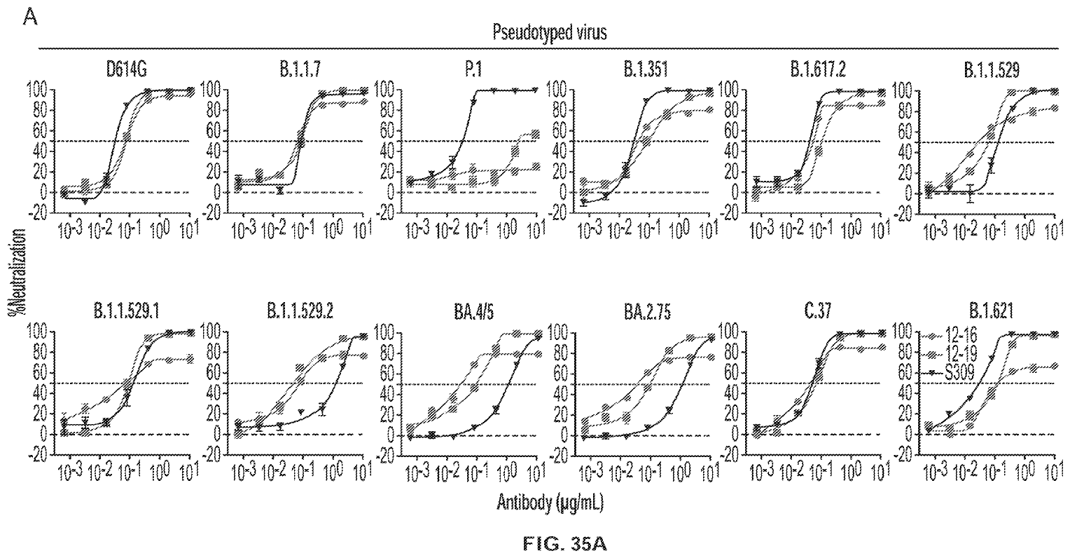

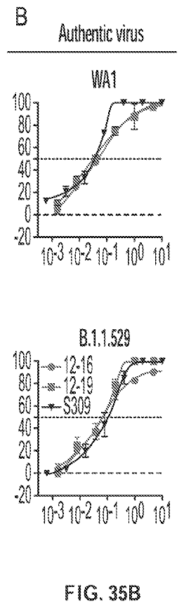

FIGS. 35A-B show neutralization profiles of engineered monoclonal 12-16 and 12-19 against SARS-COV-2 variants. FIG. 35A shows neutralization curves of antibodies against VSV pseudotypes with SARS-COV-2 spikes. FIG. 35B shows neutralization curves of antibodies against authentic D614G and B.1.1.529 viruses. Data are shown as mean±SD of three technical replicates.

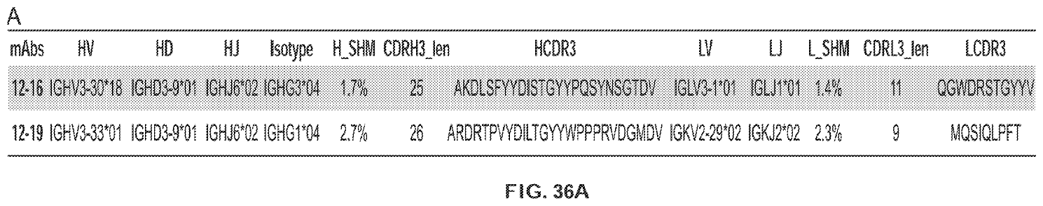

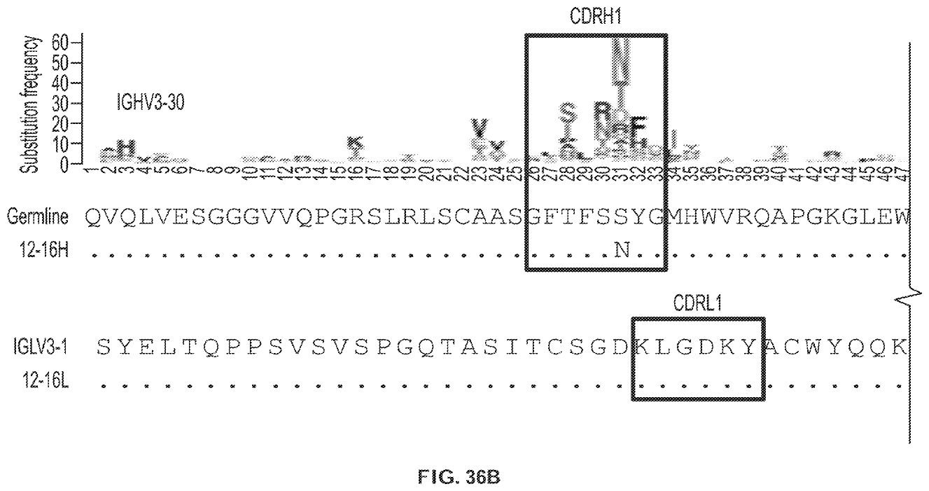

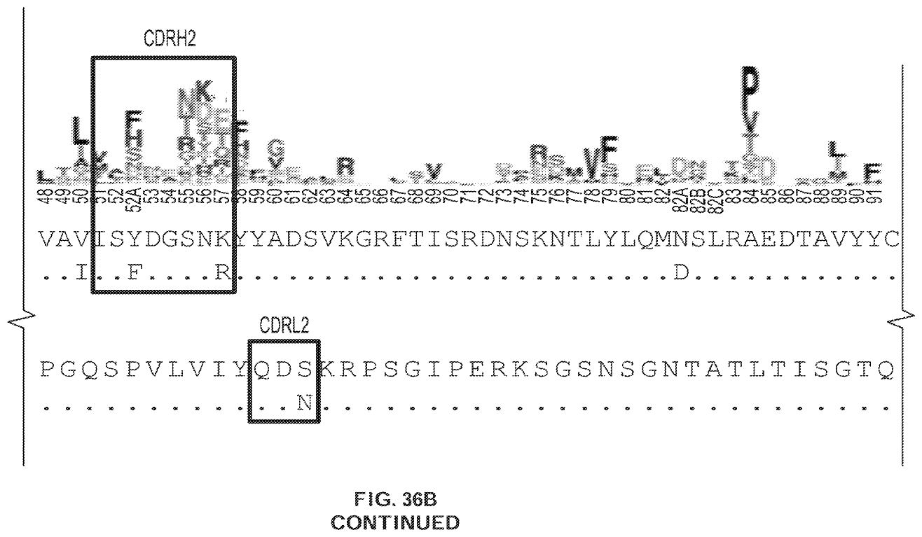

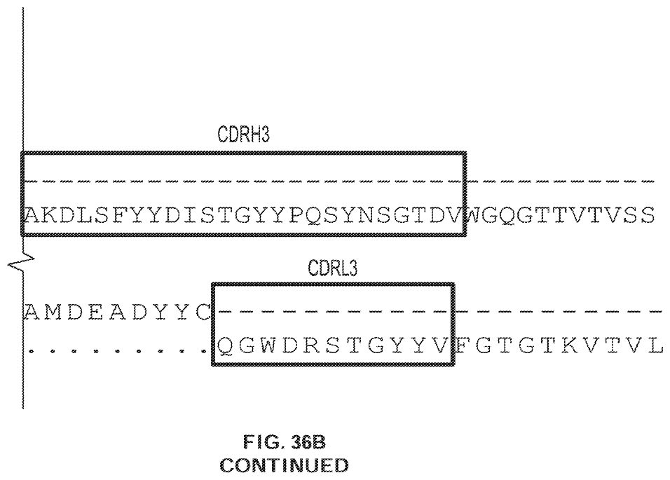

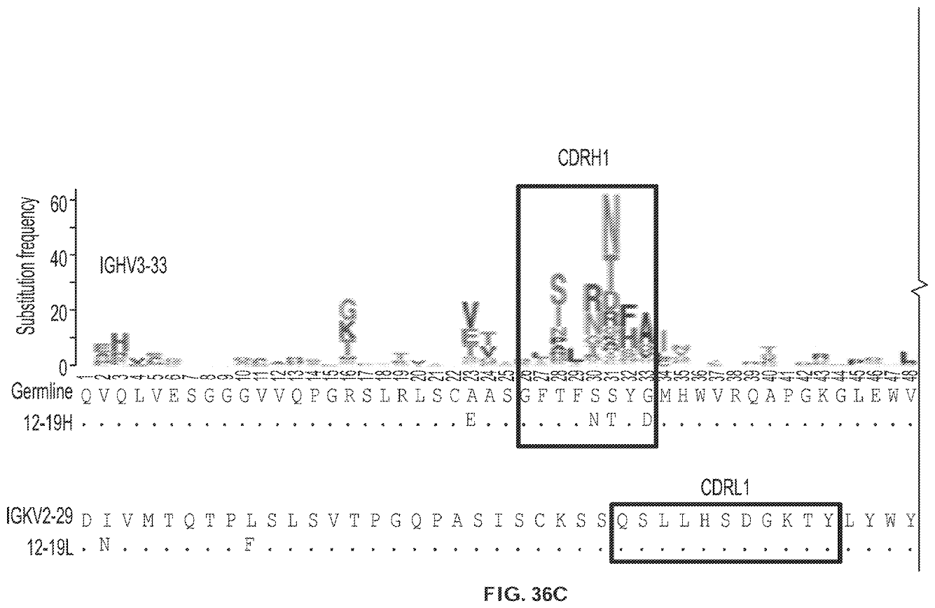

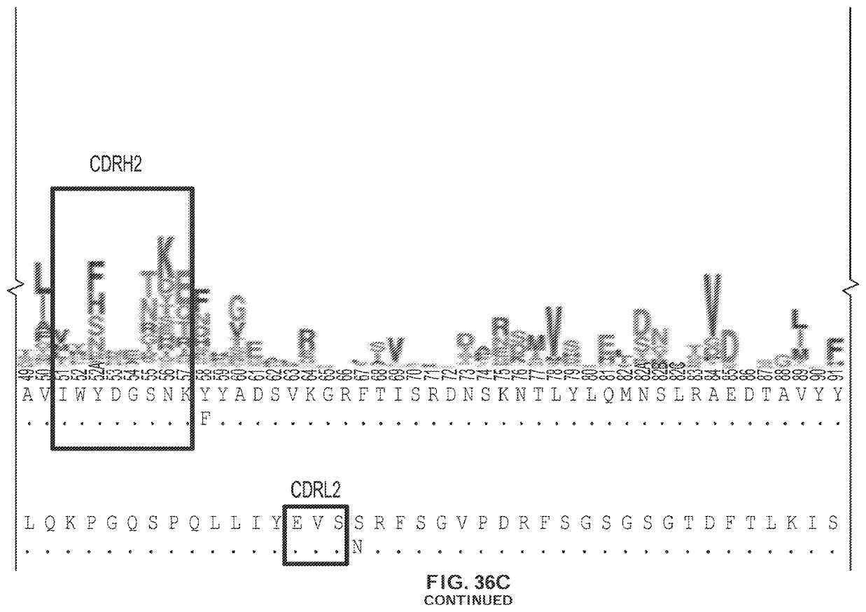

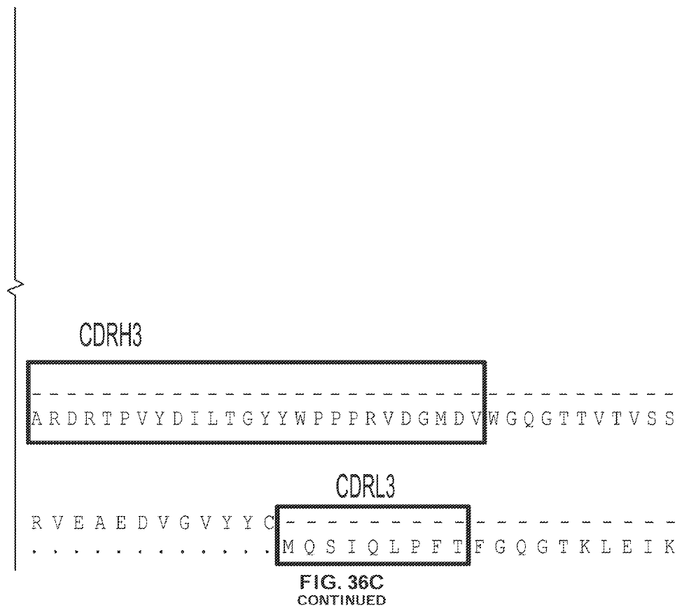

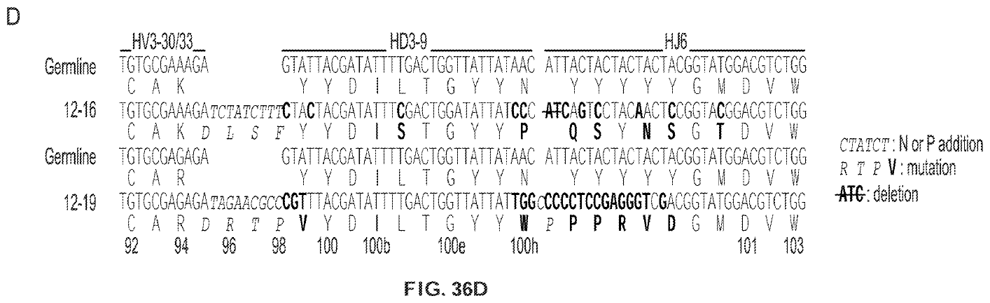

FIGS. 36A-D show genetic analysis for engineered monoclonal antibodies 12-16 and 12-19. FIG. 36A shows germline gene assignment for antibodies 12-16 and 12-19. FIG. 36B shows gene specific substitution profile (GSSP) for antibody 12-16, the dots represent the conserved residues in antibody sequence compared with germline gene, the CDRs were highlighted by rectangles. FIG. 36C shows gene specific substitution profile (GSSP) for antibody 12-19, the dots represent the conserved residues in antibody sequence compared with germline gene, the CDRs were highlighted by rectangles. FIG. 36D shows CDRH3 VDJ junction analysis for 12-16 and 12-19. Germline nucleotide and amino acid residues are shown in black with the corresponding junctions colored in light blue. Somatic hypermutations are colored in red. Nucleotides deleted by exonuclease trimming are indicated with strikethrough. The blue nucleotides represent the N and P nucleotide additions at the junctions.

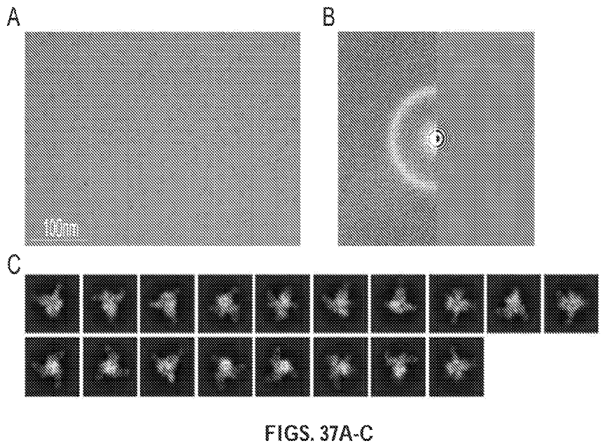

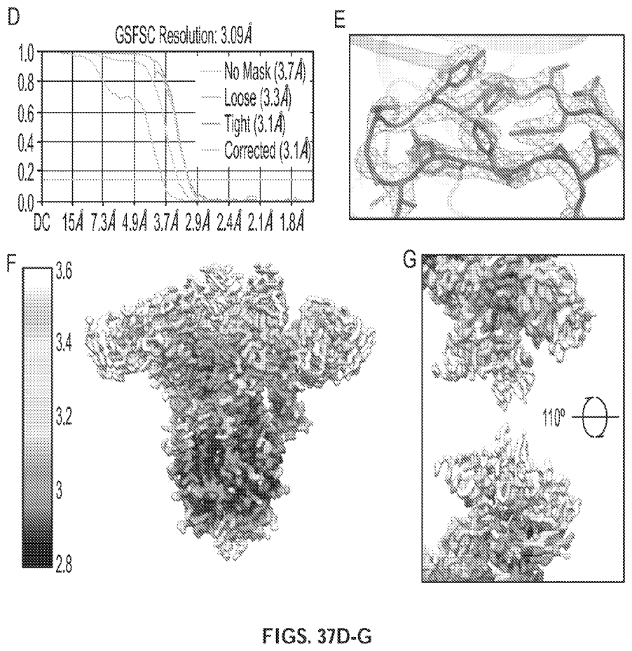

FIG. 37A-G show Cryo-EM data processing for engineered monoclonal antibody 12-16 in complex with the SARS-COV-2 D614G spike trimer. FIG. 37A shows a representative micrograph showing spike particle distribution in ice. FIG. 37B shows a micrograph power spectrum (left) with contrast transfer function (CTF) fit (right). FIG. 37C shows selected 2D class averages showing initial spike particle set. FIG. 37D shows a global refinement Fourier Shell Correlation (FSC) curve showing overall resolution. FIG. 37E shows a map density shown as a mesh for antibody CDRH3 loop showing side chain fits. FIG. 37F shows local resolution mapped onto global refinement reconstruction. FIG. 37G shows local resolution for the antibody 12-16 interface, shown from the top and the bottom.

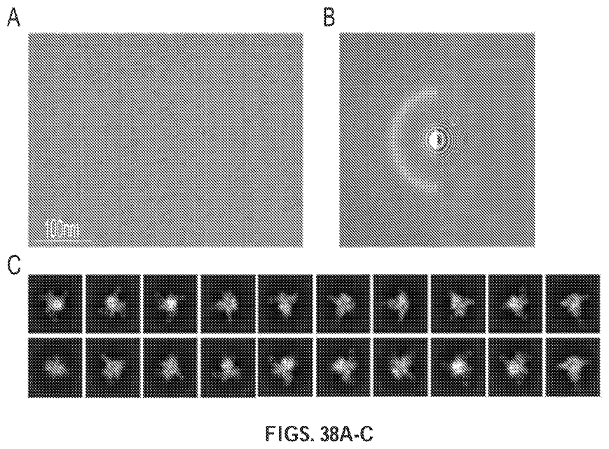

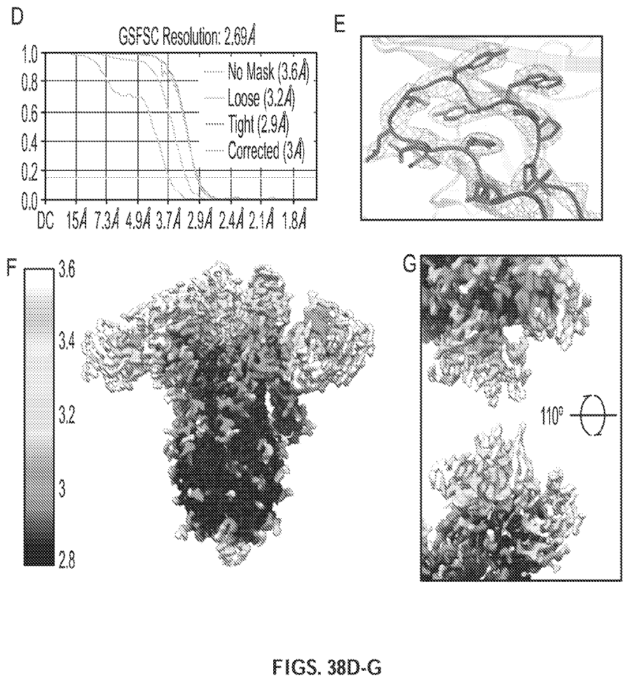

FIGS. 38A-G show Cryo-EM data processing for engineered antibody 12-19 in complex with SARS-COV-2 D614G spike trimer. FIG. 38A shows a representative micrograph showing spike particle distribution in ice. FIG. 38B shows a micrograph power spectrum (left) with contrast transfer function (CTF) fit (right). FIG. 38C shows selected 2D class averages showing initial spike particle set. FIG. 38D shows a global refinement Fourier Shell Correlation (FSC) curve showing overall resolution. FIG. 38E shows a map density shown as mesh for antibody CDRH3 loop showing side chain fits. FIG. 38F shows local resolution mapped onto global refinement reconstruction. FIG. 38G shows local resolution for the antibody 12-19 interface, shown from the top and the bottom.

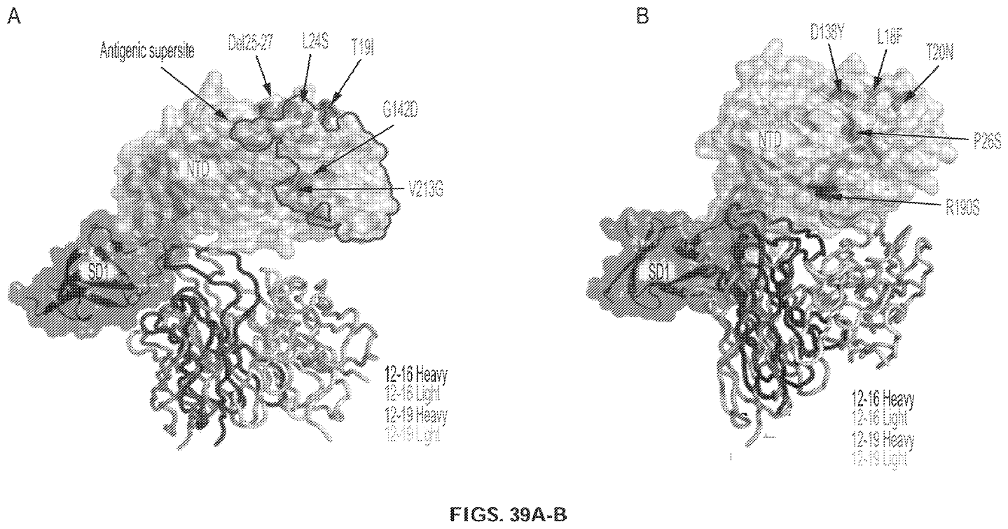

FIGS. 39A-B shows the location of mutations in the Omicron BA.2 and P.1 variants in complex with engineered monoclonal antibodies 12-16 and 12-19. FIG. 39A shows antibodies 12-16 and 12-19 in complex with NTD and SDI of a spike protein. The red spheres represent the mutations in the Omicron BA.2 variant, the NTD antigenic supersite is outlined in cyan. FIG. 39B shows antibodies 12-16 and 12-19 in complex with NTD and SD1 of a spike protein. The red spheres represent the mutations in the P.1 variant.

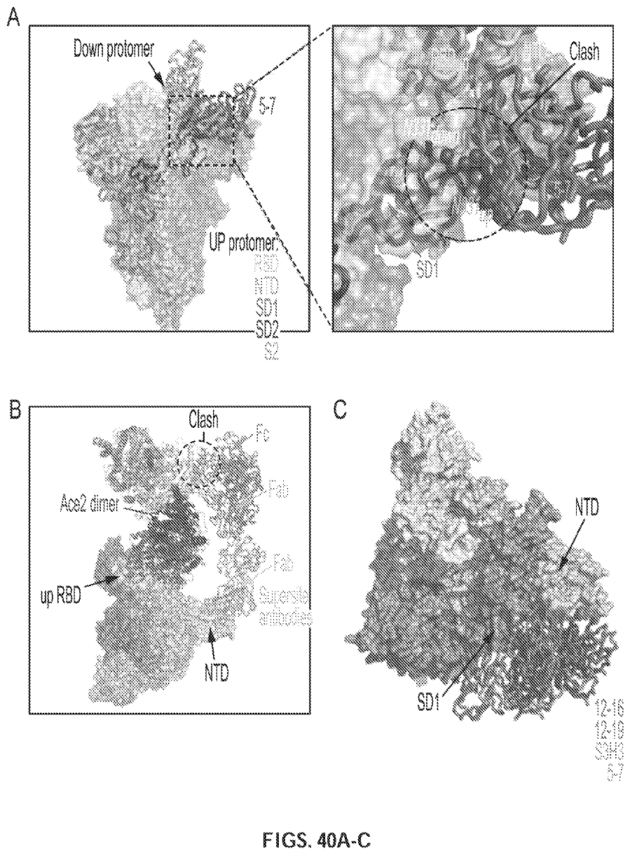

FIGS. 40A-C show structural evidence for antibody neutralization mechanisms. FIG. 40A shows superpose of RBD up and down protomers. The surface shows the RBD down protomer and the ribbon shows the RBD up protomer (PDB: 7krr). FIG. 40B shows a structural model showing that the Fc or Fab of NTD supersite antibody clash with dimer hACE2. The full IgG structure was made by superimposing the heavy chains of the crystal IgG structure (PDB: 1IHZH) with the supersite antibody (PDB: 7L2F). FIG. 40C shows a comparison of antibodies 12-16 and 12-19 with NTD antibody 5-7 and SDI antibody S3H3.

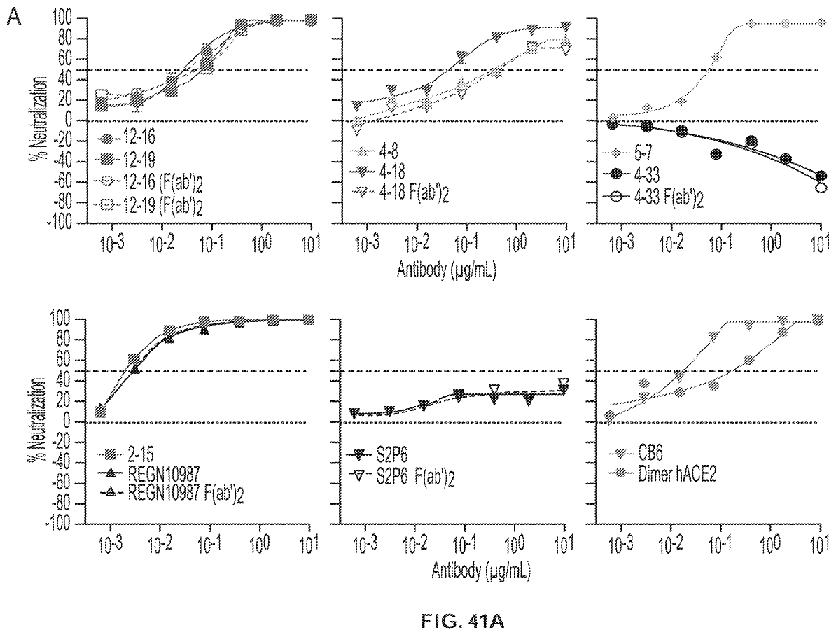

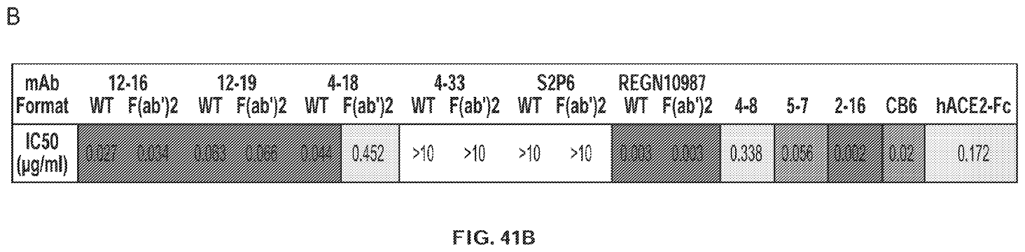

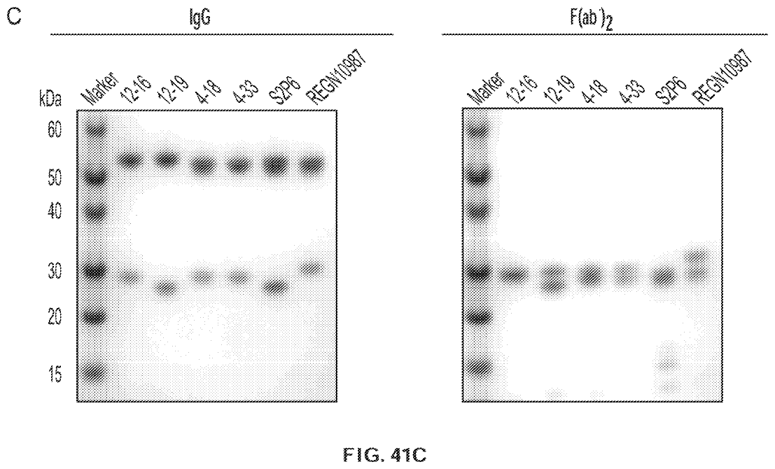

FIGS. 41A-C show neutralization of VSV pseudotyped with SARS-COV-2 D614G spike protein by the selected IgG (WT) or F(ab′) 2 format. FIG. 41A shows neutralization curves of the selected IgG and F(ab′) 2 format against VSV pseudotypes with SARS-COV-2 D614G spike protein. Data are shown as mean±SD of three technical replicates. FIG. 41B shows mAbs 50% inhibitory concentration (IC₅₀) for SARS-COV-2 D614G spike protein. Data are shown as mean±SD of three technical replicates. FIG. 41C shows SDS-PAGE results for purity determination of purified IgG (left panel) and F(ab′) 2 (right panel).

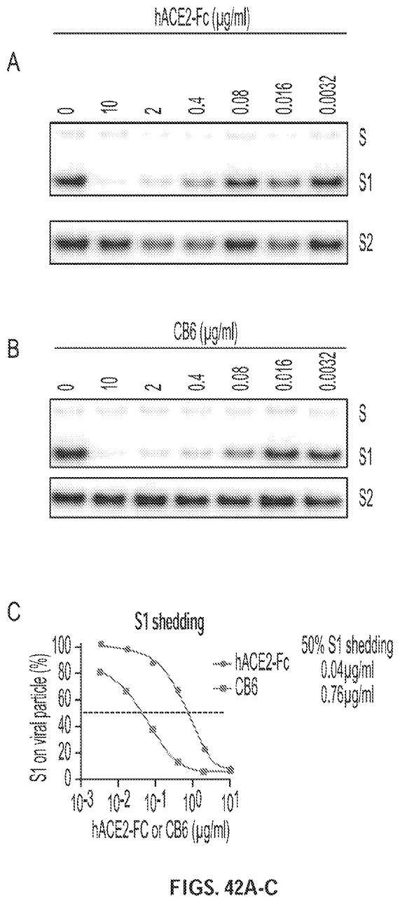

FIGS. 42A-C show that soluble ACE2 or CB6 induced the shedding of S1 from VSV particles pseudotyped with the SARS-COV-2 D614G spike glycoproteins. FIGS. 42A and B show that VSV particles pseudotyped with the variant SARS-COV-2 D614G S glycoproteins were incubated with the indicated concentrations of soluble ACE2 (sACE2) or CB6 for one and half hours at 37° C., respectively. Pelleted VSV particles were analyzed by Western blotting with antibodies against S1, S2. FIG. 42C shows the intensities of the S1 and S2 glycoprotein bands in (A) and (B) were measured and the S1/S2 ratios for each concentration of sACE2 or CB6 are shown.

FIGS. 43A-B show binding kinetics and affinities of selected antibodies. FIG. 43A shows that binding of antibodies to SARS-COV-2 D614G spike protein was determined by surface plasmon resonance (SPR). The spike protein-bound sensors were incubated with six different concentrations of antibodies. Kinetic data from one representative experiment were fit to a 1:1 binding model. FIG. 43B shows summary of SPR kinetic and affinity measurements.

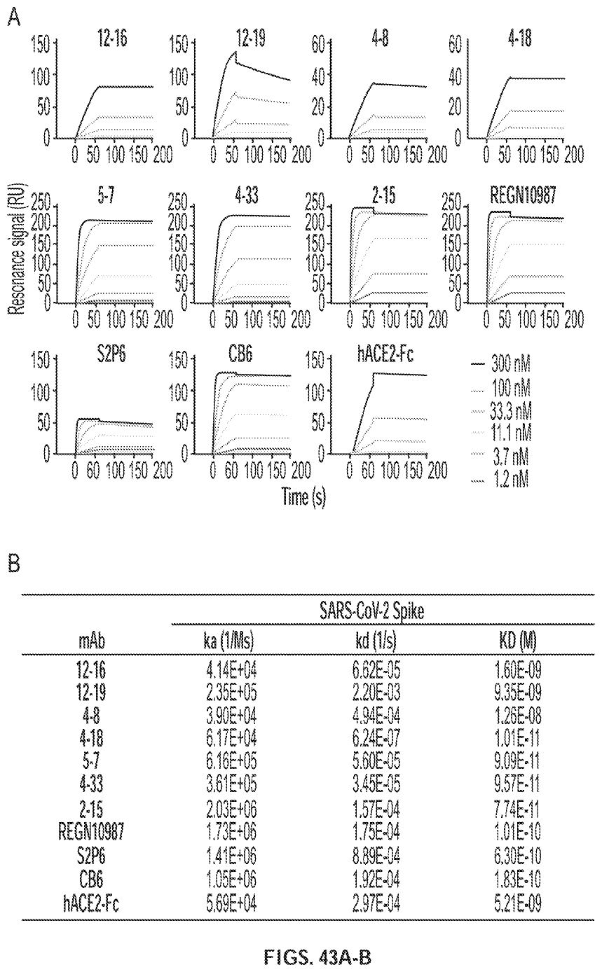

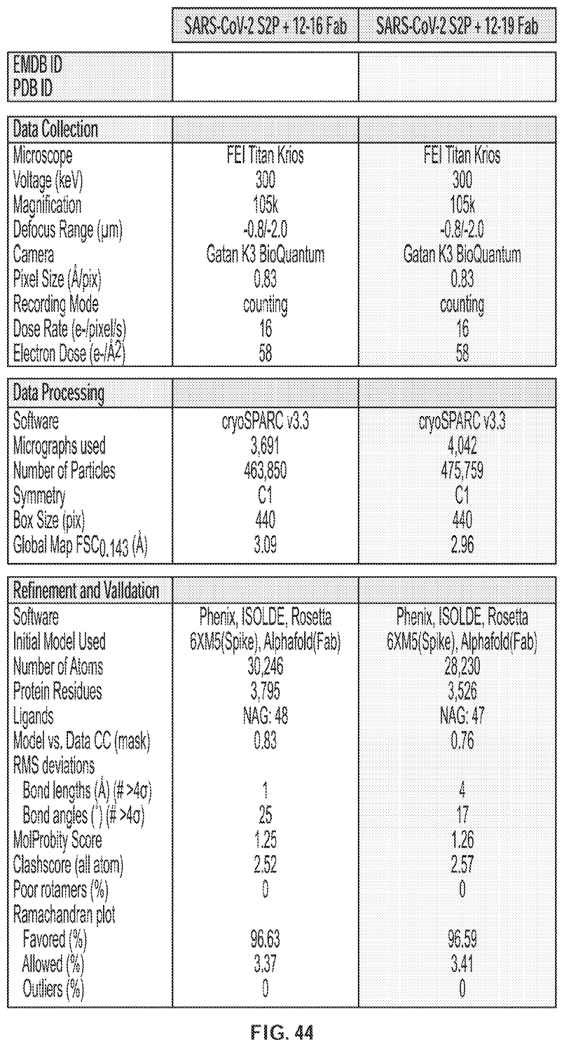

FIG. 44 shows CryoEM data collection and model refinement.

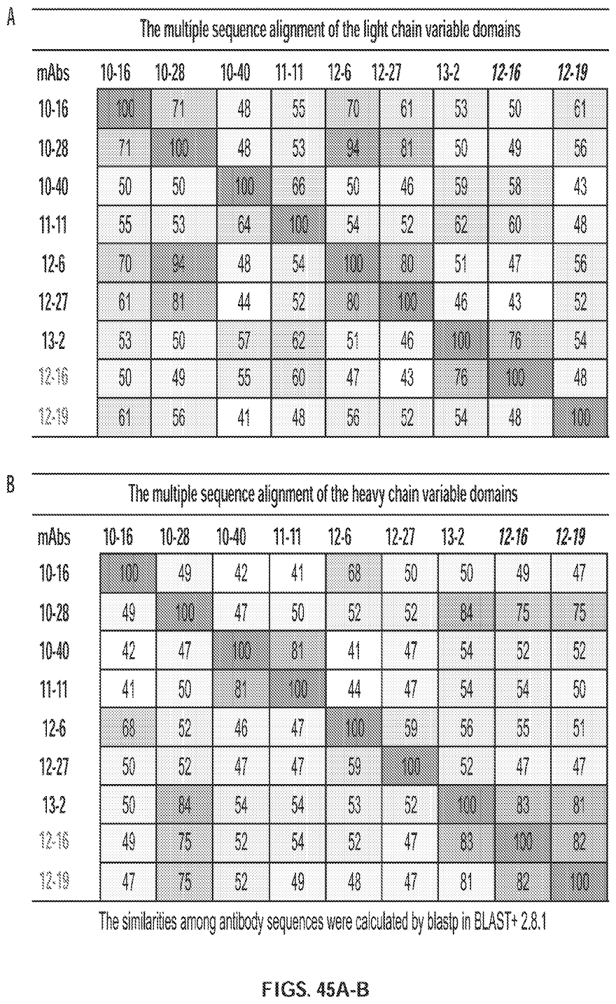

FIGS. 45A-B show sequence alignment monoclonal antibodies disclosed herein. FIG. 45A shows multiple sequence alignment of the light chain variable domains of antibodies disclosed herein. FIG. 45B shows multiple sequence alignment of the heavy chain variable domains of antibodies disclosed herein.

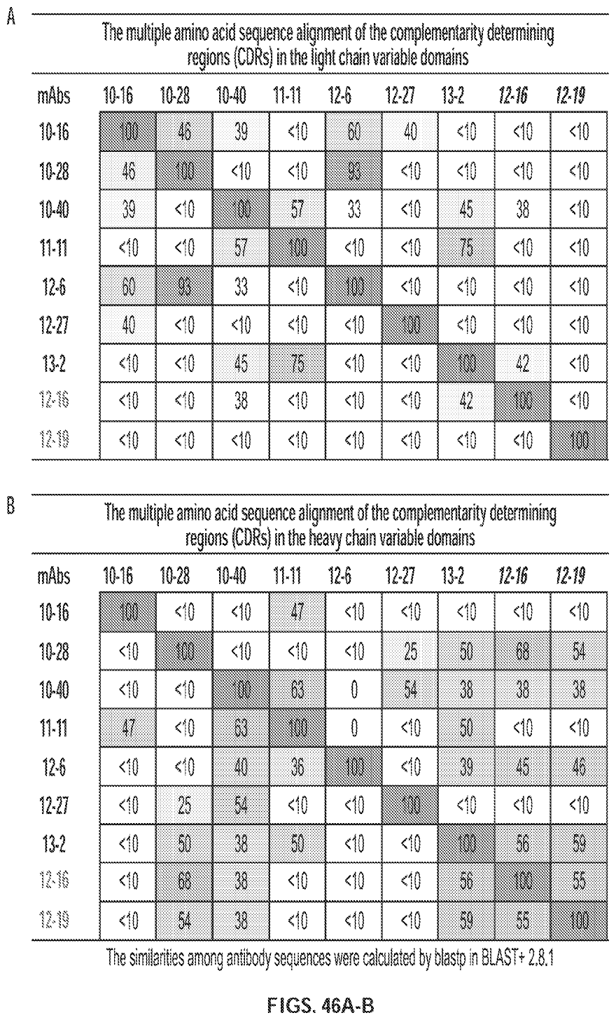

FIGS. 46A-B shows identification of the complementarity determining regions (CDRs) of monoclonal antibodies. FIG. 46A shows sequence alignment of CDRs in the light chain variable domains. FIG. 46B shows sequence alignment of CDRs in the heavy chain variable domains.

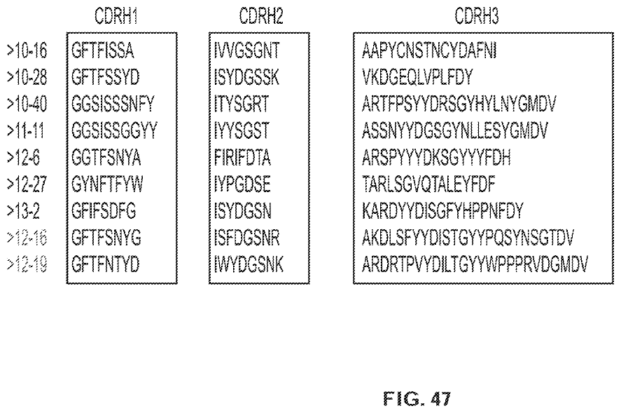

FIG. 47 shows the amino acid sequences of the CDRs in heavy chains.

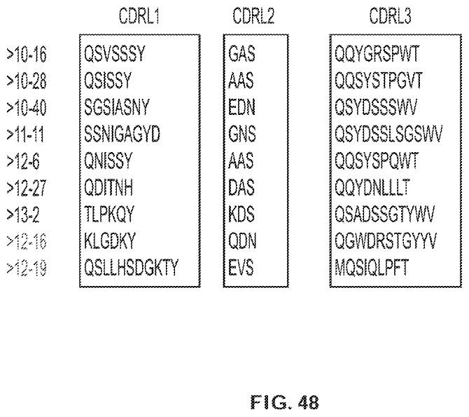

FIG. 48 shows the amino acid sequences of the CDRs in light chains.

What is claimed is:

1. A engineered monoclonal antibody, or a functional fragment thereof, selectively binding and neutralizing a at least a portion of a virus, wherein the antibody, or functional fragment thereof, comprises an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1, at least 60%, 62%, 65%, 67%, 70%, 72%, 75%, 77%, 80%, 82%, 85%, 87%, 90%, 92%, 95%, 97%, 98%, 99% identical to SEQ ID NO: 2, at least 60%, 62%, 65%, 67%, 70%, 72%, 75%, 77%, 80%, 82%, 85%, 87%, 90%, 92%, 95%, 97%, 98%, 99% identical to SEQ ID NO: 3, at least 60%, 62%, 65%, 67%, 70%, 72%, 75%, 77%, 80%, 82%, 85%, 87%, 90%, 92%, 95%, 97%, 98%, 99% identical to SEQ ID NO: 4, at least 60%, 62%, 65%, 67%, 70%, 72%, 75%, 77%, 80%, 82%, 85%, 87%, 90%, 92%, 95%, 97%, 98%, 99% identical to SEQ ID NO: 5, at least 60%, 62%, 65%, 67%, 70%, 72%, 75%, 77%, 80%, 82%, 85%, 87%, 90%, 92%, 95%, 97%, 98%, 99% identical to SEQ ID NO: 6, at least 60%, 62%, 65%, 67%, 70%, 72%, 75%, 77%, 80%, 82%, 85%, 87%, 90%, 92%, 95%, 97%, 98%, 99% identical to SEQ ID NO: 7, at least 60%, 62%, 65%, 67%, 70%, 72%, 75%, 77%, 80%, 82%, 85%, 87%, 90%, 92%, 95%, 97%, 98%, 99% identical to SEQ ID NO: 8, at least 60%, 62%, 65%, 67%, 70%, 72%, 75%, 77%, 80%, 82%, 85%, 87%, 90%, 92%, 95%, 97%, 98%, 99% identical to SEQ ID NO: 9, at least 60%, 62%, 65%, 67%, 70%, 72%, 75%, 77%, 80%, 82%, 85%, 87%, 90%, 92%, 95%, 97%, 98%, 99% identical to SEQ ID NO: 10, at least 60%, 62%, 65%, 67%, 70%, 72%, 75%, 77%, 80%, 82%, 85%, 87%, 90%, 92%, 95%, 97%, 98%, 99% identical to SEQ ID NO: 11, at least 60%, 62%, 65%, 67%, 70%, 72%, 75%, 77%, 80%, 82%, 85%, 87%, 90%, 92%, 95%, 97%, 98%, 99% identical to SEQ ID NO: 12, at least 60%, 62%, 65%, 67%, 70%, 72%, 75%, 77%, 80%, 82%, 85%, 87%, 90%, 92%, 95%, 97%, 98%, 99% identical to SEQ ID NO: 13, or at least 60%, 62%, 65%, 67%, 70%, 72%, 75%, 77%, 80%, 82%, 85%, 87%, 90%, 92%, 95%, 97%, 98%, 99% identical to SEQ ID NO: 14.

2. The antibody or fragment of claim 1, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2.

3. The antibody or fragment of claim 1, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4.

4. The antibody or fragment of claim 1, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6.

5. The antibody or fragment of claim 1, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8.

6. The antibody or fragment of claim 1, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

7. The antibody or fragment of claim 1, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12.

8. The antibody or fragment of claim 1, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

9. The antibody or fragment of claim 1, wherein the antibody or functional fragment thereof comprises a heavy chain comprising an amino acid sequence identical to SEQ ID NO: 1 and a light chain comprising an amino acid sequence identical to SEQ ID NO: 2.

10. The antibody or fragment of claim 1, wherein the antibody or functional fragment thereof comprises a heavy chain comprising an amino acid sequence identical to SEQ ID NO: 3 and a light chain comprising an amino acid sequence identical to SEQ ID NO: 4.

11. The antibody or fragment of claim 1, wherein the antibody or functional fragment thereof comprises a heavy chain comprising an amino acid sequence identical to SEQ ID NO: 5 and a light chain comprising an amino acid sequence identical SEQ ID NO: 6.

12. The antibody or fragment of claim 1, wherein the antibody or functional fragment thereof comprises a heavy chain comprising an amino acid sequence identical to SEQ ID NO: 7 and a light chain comprising an amino acid sequence identical to SEQ ID NO: 8.

13. The antibody or fragment of claim 1, wherein the antibody or functional fragment thereof comprises a heavy chain comprising an amino acid sequence identical to SEQ ID NO: 9 and a light chain comprising an amino acid sequence identical to SEQ ID NO: 10.

14. The antibody or fragment of claim 1, wherein the antibody or functional fragment thereof comprises a heavy chain comprising an amino acid sequence identical to SEQ ID NO: 11 and a light chain comprising an amino acid sequence identical to SEQ ID NO: 12.

15. The antibody or fragment of claim 1, wherein the antibody or functional fragment thereof comprises a heavy chain comprising an amino acid sequence identical to SEQ ID NO: 13 and a light chain comprising an amino acid sequence identical to SEQ ID NO: 14.

16. The antibody or fragment of claim 1, wherein the antibody or functional fragment thereof comprises a heavy chain comprising an amino acid sequence comprising the complementarity-determining regions (CDRs) of SEQ ID NO: 1 and a light chain comprising an amino acid sequence comprising the complementarity-determining regions (CDRs) of SEQ ID NO: 2.

17. The antibody or fragment of claim 1, wherein the antibody or functional fragment thereof comprises a heavy chain comprising an amino acid sequence comprising the complementarity-determining regions (CDRs) of SEQ ID NO: 3 and a light chain comprising an amino acid sequence comprising the complementarity-determining regions (CDRs) of SEQ ID NO: 4.

18. The antibody or fragment of claim 1, wherein the antibody or functional fragment thereof comprises a heavy chain comprising an amino acid sequence comprising the complementarity-determining regions (CDRs) of SEQ ID NO: 5 and a light chain comprising an amino acid sequence comprising the complementarity-determining regions (CDRs) of SEQ ID NO: 6.

19. The antibody or fragment of claim 1, wherein the antibody or functional fragment thereof comprises a heavy chain comprising an amino acid sequence comprising the complementarity-determining regions (CDRs) of SEQ ID NO: 7 and a light chain comprising an amino acid sequence comprising the complementarity-determining regions (CDRs) of SEQ ID NO: 8.

20. The antibody or fragment of claim 1, wherein the antibody or functional fragment thereof comprises a heavy chain comprising an amino acid sequence comprising the complementarity-determining regions (CDRs) of SEQ ID NO: 9 and a light chain comprising an amino acid sequence comprising the complementarity-determining regions (CDRs) of SEQ ID NO: 10.

21. The antibody or fragment of claim 1, wherein the antibody or functional fragment thereof comprises a heavy chain comprising an amino acid sequence comprising the complementarity-determining regions (CDRs) of SEQ ID NO: 11 and a light chain comprising an amino acid sequence comprising the complementarity-determining regions (CDRs) of SEQ ID NO: 12.

22. The antibody or fragment of claim 1, wherein the antibody or functional fragment thereof comprises a heavy chain comprising an amino acid sequence comprising the complementarity-determining regions (CDRs) of SEQ ID NO: 13 and a light chain comprising an amino acid sequence comprising the complementarity-determining regions (CDRs) of SEQ ID NO: 14.

23. The antibody or fragment of claim 1, wherein the virus is a coronavirus.

24. The antibody or fragment of claim 23, wherein the coronavirus is selected from the coronaviruses consisting of SARS-COV-2, SARS-COV, MERS-COV, 229E, NL63, OC43, BM-4831, BtKY72, Rf1, RmYN02, Rs4081, Yunnan2011, ZC45, As6526, Rs4237, Rs4081, and HKU1.

25. The antibody or fragment of claim 24, wherein the coronavirus is a SARS-COV-2 virus.

26. The antibody or fragment of claim 25, wherein the SARS-COV-2 virus is a wild-type strain.

27. The antibody or fragment of claim 25, wherein the SARS-COV-2 virus is a SARS-CoV-2 D614G strain.

28. The antibody or fragment of claim 25, wherein the SARS-COV-2 virus is a WA1 strain.

29. The antibody or fragment of claim 25, wherein the SARS-COV-2 virus is a B.1.1.7 strain.

30. The antibody or fragment of claim 25, wherein the SARS-COV-2 virus is a B.1.351 strain.

31. The antibody or fragment of claim 25, wherein the SARS-COV-2 virus is a P.1 strain.

32. The antibody or fragment of claim 25, wherein the SARS-COV-2 virus is a B.1.526 strain.

33. The antibody or fragment of claim 25, wherein the SARS-COV-2 virus is a B.1.429 strain.

34. The antibody or fragment of claim 25, wherein the SARS-COV-2 virus is a B.1.617.1 strain.

35. The antibody or fragment of claim 25, wherein the SARS-COV-2 virus is a B.1.1.1 strain.

36. The antibody or fragment of claim 25, wherein the SARS-COV-2 virus is a B.1.617.2 strain.

37. The antibody or fragment of claim 25, wherein the SARS-COV-2 virus is a B.1.526-E484K strain.

38. The antibody or fragment of claim 25, wherein the SARS-COV-2 virus is a V236P12 strain.

39. The antibody or fragment of claim 24, wherein the coronavirus is a SARS-COV virus.

40. The antibody or fragment of claim 24, wherein the coronavirus is a MERS-COV virus.

41. The antibody or fragment of claim 24, wherein the coronavirus is a NL63 virus.

42. The antibody or fragment of claim 23, wherein the coronavirus is a GD-Pangolin virus.

43. The antibody or fragment of claim 23, wherein the coronavirus is a GX-Pangolin virus.

44. The antibody or fragment of claim 23, wherein the coronavirus is a RaTG13 virus.

45. The antibody or fragment of claim 23, wherein the coronavirus is a WIV1 virus.

46. The antibody or fragment of claim 23, wherein the coronavirus is a SHC014 virus.

47. The antibody or fragment of claim 23, wherein the coronavirus is a LYRa11 virus.

48. The antibody or fragment of claim 23, wherein the coronavirus is a Rs7327 virus.

49. The antibody or fragment of claim 23, wherein the coronavirus is a Rs4231 virus.

50. The antibody of claim 23, wherein the coronavirus is a Rs4084 virus.

51. A method for treating a subject having a viral infection or preventing a subject from developing a viral infection, the method comprising administering to the subject a therapeutically effective amount of a composition comprising an engineered monoclonal antibody or a functional fragment thereof.

52. The method of claim 51, wherein the antibody comprises an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1, at least 60%, 62%, 65%, 67%, 70%, 72%, 75%, 77%, 80%, 82%, 85%, 87%, 90%, 92%, 95%, 97%, 98%, 99% identical to SEQ ID NO: 2, at least 60%, 62%, 65%, 67%, 70%, 72%, 75%, 77%, 80%, 82%, 85%, 87%, 90%, 92%, 95%, 97%, 98%, 99% identical to SEQ ID NO: 3, at least 60%, 62%, 65%, 67%, 70%, 72%, 75%, 77%, 80%, 82%, 85%, 87%, 90%, 92%, 95%, 97%, 98%, 99% identical to SEQ ID NO: 4, at least 60%, 62%, 65%, 67%, 70%, 72%, 75%, 77%, 80%, 82%, 85%, 87%, 90%, 92%, 95%, 97%, 98%, 99% identical to SEQ ID NO: 5, at least 60%, 62%, 65%, 67%, 70%, 72%, 75%, 77%, 80%, 82%, 85%, 87%, 90%, 92%, 95%, 97%, 98%, 99% identical to SEQ ID NO: 6, at least 60%, 62%, 65%, 67%, 70%, 72%, 75%, 77%, 80%, 82%, 85%, 87%, 90%, 92%, 95%, 97%, 98%, 99% identical to SEQ ID NO: 7, at least 60%, 62%, 65%, 67%, 70%, 72%, 75%, 77%, 80%, 82%, 85%, 87%, 90%, 92%, 95%, 97%, 98%, 99% identical to SEQ ID NO: 8, at least 60%, 62%, 65%, 67%, 70%, 72%, 75%, 77%, 80%, 82%, 85%, 87%, 90%, 92%, 95%, 97%, 98%, 99% identical to SEQ ID NO: 9, at least 60%, 62%, 65%, 67%, 70%, 72%, 75%, 77%, 80%, 82%, 85%, 87%, 90%, 92%, 95%, 97%, 98%, 99% identical to SEQ ID NO: 10, at least 60%, 62%, 65%, 67%, 70%, 72%, 75%, 77%, 80%, 82%, 85%, 87%, 90%, 92%, 95%, 97%, 98%, 99% identical to SEQ ID NO: 11, at least 60%, 62%, 65%, 67%, 70%, 72%, 75%, 77%, 80%, 82%, 85%, 87%, 90%, 92%, 95%, 97%, 98%, 99% identical to SEQ ID NO: 12, at least 60%, 62%, 65%, 67%, 70%, 72%, 75%, 77%, 80%, 82%, 85%, 87%, 90%, 92%, 95%, 97%, 98%, 99% identical to SEQ ID NO: 13, or at least 60%, 62%, 65%, 67%, 70%, 72%, 75%, 77%, 80%, 82%, 85%, 87%, 90%, 92%, 95%, 97%, 98%, 99% identical to SEQ ID NO: 14.

53. The method of claim 52, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2.

54. The method of claim 52, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4.

55. The method of claim 52, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6.

56. The method of claim 52, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8.

57. The method of claim 52, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

58. The method of claim 52, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12.

59. The method of claim 52, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

60. The method of claim 52, wherein the antibody or functional fragment thereof comprises a heavy chain comprising an amino acid sequence identical to SEQ ID NO: 1 and a light chain comprising an amino acid sequence identical to SEQ ID NO: 2.

61. The method of claim 52, wherein the antibody or functional fragment thereof comprises a heavy chain comprising an amino acid sequence identical to SEQ ID NO: 3 and a light chain comprising an amino acid sequence identical to SEQ ID NO: 4.

62. The method of claim 52, wherein the antibody or functional fragment thereof comprises a heavy chain comprising an amino acid sequence identical to SEQ ID NO: 5 and a light chain comprising an amino acid sequence identical to SEQ ID NO: 6.

63. The method of claim 52, wherein the antibody or functional fragment thereof comprises a heavy chain comprising an amino acid sequence identical to SEQ ID NO: 7 and a light chain comprising an amino acid sequence identical to SEQ ID NO: 8.

64. The method of claim 52, wherein the antibody or functional fragment thereof comprises a heavy chain comprising an amino acid sequence identical to SEQ ID NO: 9 and a light chain comprising an amino acid sequence identical to SEQ ID NO: 10.

65. The method of claim 52, wherein the antibody or functional fragment thereof comprises a heavy chain comprising an amino acid sequence identical to SEQ ID NO: 11 and a light chain comprising an amino acid sequence identical to SEQ ID NO: 12.

66. The method of claim 52, wherein the antibody or functional fragment thereof comprises a heavy chain comprising an amino acid sequence identical to SEQ ID NO: 13 and a light chain comprising an amino acid sequence identical to SEQ ID NO: 14.

67. The method of claim 52, wherein the antibody or functional fragment thereof comprises a heavy chain comprising an amino acid sequence comprising the complementarity-determining regions (CDRs) of SEQ ID NO: 1 and a light chain comprising an amino acid sequence comprising the complementarity-determining regions (CDRs) of SEQ ID NO: 2.

68. The method of claim 52, wherein the antibody or functional fragment thereof comprises a heavy chain comprising an amino acid sequence comprising the complementarity-determining regions (CDRs) of SEQ ID NO: 3 and a light chain comprising an amino acid sequence comprising the complementarity-determining regions (CDRs) of SEQ ID NO: 4.

69. The method of claim 52, wherein the antibody or functional fragment thereof comprises a heavy chain comprising an amino acid sequence comprising the complementarity-determining regions (CDRs) of SEQ ID NO: 5 and a light chain comprising an amino acid sequence comprising the complementarity-determining regions (CDRs) of SEQ ID NO: 6.

70. The method of claim 52, wherein the antibody or functional fragment thereof comprises a heavy chain comprising an amino acid sequence comprising the complementarity-determining regions (CDRs) of SEQ ID NO: 7 and a light chain comprising an amino acid sequence comprising the complementarity-determining regions (CDRs) of SEQ ID NO: 8.

71. The method of claim 52, wherein the antibody or functional fragment thereof comprises a heavy chain comprising an amino acid sequence comprising the complementarity-determining regions (CDRs) of SEQ ID NO: 9 and a light chain comprising an amino acid sequence comprising the complementarity-determining regions (CDRs) of SEQ ID NO: 10.

72. The method of claim 52, wherein the antibody or functional fragment thereof comprises a heavy chain comprising an amino acid sequence comprising the complementarity-determining regions (CDRs) of SEQ ID NO: 11 and a light chain comprising an amino acid sequence comprising the complementarity-determining regions (CDRs) of SEQ ID NO: 12.

73. The method of claim 52, wherein the antibody or functional fragment thereof comprises a heavy chain comprising an amino acid sequence comprising the complementarity-determining regions (CDRs) of SEQ ID NO: 13 and a light chain comprising an amino acid sequence comprising the complementarity-determining regions (CDRs) of SEQ ID NO: 14.

74. The method of claim 51, wherein the virus is a coronavirus.

75. The method of claim 74, wherein the coronavirus is selected from the strains consisting of SARS-COV-2, SARS-COV, MERS-COV, 229E, NL63, OC43, BM-4831, BtKY72, Rf1, RmYN02, Rs4081, Yunnan2011, ZC45, As6526, Rs4237, Rs4081, and HKU1.

76. The method of claim 75, wherein the coronavirus is a SARS-COV-2 virus.

77. The method of claim 76, wherein the SARS-COV-2 virus is a wild-type strain.

78. The method of claim 76, wherein the SARS-COV-2 virus is a SARS-COV-2 D614G strain.

79. The method of claim 76, wherein the SARS-COV-2 virus is a WA1 strain.

80. The method of claim 76, wherein the SARS-COV-2 virus is a B.1.1.7 strain.

81. The method of claim 76, wherein the SARS-COV-2 virus is a B.1.351 strain.

82. The method of claim 76, wherein the SARS-COV-2 virus is a P.1 strain.

83. The method of claim 76, wherein the SARS-COV-2 virus is a B.1.526 strain.

84. The method of claim 76, wherein the SARS-COV-2 virus is a B.1.429 strain.

85. The method of claim 76, wherein the SARS-COV-2 virus is a B.1.617.1 strain.

86. The method of claim 76, wherein the SARS-COV-2 virus is a B.1.1.1 strain.

87. The method of claim 76, wherein the SARS-COV-2 virus is a B.1.617.2 strain.

88. The method of claim 76, wherein the SARS-COV-2 virus is a B.1.526-E484K strain.

89. The method of claim 76, wherein the SARS-COV-2 virus is a V236P12 strain.

90. The method of claim 75, wherein the coronavirus is a SARS-COV virus.

91. The method of claim 75, wherein the coronavirus is a MERS-COV virus.

92. The method of claim 75, wherein the coronavirus is a NL63 virus.

93. The method of claim 74, wherein the coronavirus is a GD-Pangolin virus.

94. The method of claim 74, wherein the coronavirus is a GX-Pangolin virus.

95. The method of claim 74, wherein the coronavirus is a RaTG13 virus.

96. The method of claim 74, wherein the coronavirus is a WIV1 virus.

97. The method of claim 74, wherein the coronavirus is a SHC014 virus.

98. The method of claim 74, wherein the coronavirus is a LYRa11 virus.

99. The method of claim 74, wherein the coronavirus is a Rs7327 virus.

100. The method of claim 74, wherein the coronavirus is a Rs4231 virus.

101. The method of claim 74, wherein the coronavirus is a Rs4084 virus.

102. The method of claim 51, wherein the composition comprises a cocktail of two or more engineered monoclonal antibodies.

103. The method of claim 51, wherein the composition further comprises an antiviral agent.

104. A method of producing engineered monoclonal antibodies, the method comprising:
a) isolating a biological sample from a subject infected with a virus;
b) determining whether the biological sample has neutralizing activity against the virus;
c) isolating peripheral blood mononuclear cells (PMBCs) from the subjects whose biological sample has neutralizing activity in step b);
d) isolating coronavirus S protein trimer-specific memory single B cells from the PMBCs in c);
e) recovering one of more monoclonal antibody sequences selective for a coronavirus S protein trimer from the B cells;
f) determining the sequence encoding the one or more monoclonal antibodies recovered from the single B cells;
g) cloning the sequence encoding the one or more monoclonal antibodies into one or more expression vectors;
h) expressing one or more engineered monoclonal antibody in vitro; and
i) purifying the one or more engineered monoclonal antibody.

105. The method of claim 104, wherein the biological sample is plasma.

106. The method of claim 104, wherein the subject is not vaccinated against the virus.

107. The method of claim 104, wherein the subject is vaccinated against the virus.

108. The method of claim 104, wherein the determining in f) comprises high throughput sequencing.

109. The method of claim 104, wherein the virus is a coronavirus.

110. The method of claim 109, wherein the coronavirus is selected from the strains consisting of SARS-COV-2, SARS-COV, MERS-COV, 229E, NL63, OC43, BM-4831, BtKY72, Rf1, RmYN02, Rs4081, Yunnan2011, ZC45, As6526, Rs4237, Rs4081, and HKU1.

111. The method of claim 110, wherein the coronavirus is a SARS-COV-2 virus.

112. The method of claim 111, wherein the SARS-COV-2 virus is a wild-type strain.

113. The method of claim 111, wherein the SARS-COV-2 virus is a SARS-COV-2 D614G strain.

114. The method of claim 111, wherein the SARS-COV-2 virus is a WA1 strain.

115. The method of claim 111, wherein the SARS-COV-2 virus is a B.1.1.7 strain.

116. The method of claim 111, wherein the SARS-COV-2 virus is a B.1.351 strain.

117. The method of claim 111, wherein the SARS-COV-2 virus is a P.1 strain.

118. The method of claim 111, wherein the SARS-COV-2 virus is a B.1.526 strain.

119. The method of claim 111, wherein the SARS-COV-2 virus is a B.1.429 strain.

120. The method of claim 111, wherein the SARS-COV-2 virus is a B.1.617.1 strain.

121. The method of claim 111, wherein the SARS-COV-2 virus is a B.1.1.1 strain.

122. The method of claim 111, wherein the SARS-COV-2 virus is a B.1.617.2 strain.

123. The method of claim 111, wherein the SARS-COV-2 virus is a B.1.526-E484K strain.

124. The method of claim 111, wherein the SARS-COV-2 virus is a V236P12 strain.

125. The method of claim 110, wherein the coronavirus is a SARS-COV virus.

126. The method of claim 110, wherein the coronavirus is a MERS-COV virus.

127. The method of claim 110, wherein the coronavirus is a NL63 virus.

128. The method of claim 109, wherein the coronavirus is a GD-Pangolin virus.

129. The method of claim 109, wherein the coronavirus is a GX-Pangolin virus.

130. The method of claim 109, wherein the coronavirus is a RaTG13 virus.

131. The method of claim 109, wherein the coronavirus is a WIV1 virus.

132. The method of claim 109, wherein the coronavirus is a SHC014 virus.

133. The method of claim 109, wherein the coronavirus is a LYRa11 virus.

134. The method of claim 109, wherein the coronavirus is a Rs7327 virus.

135. The method of claim 109, wherein the coronavirus is a Rs4231 virus.

136. The method of claim 109, wherein the coronavirus is a Rs4084 virus.

137. An engineered monoclonal antibody, or a functional fragment thereof, wherein the antibody or fragment thereof selectively binds at least a portion of a virus.

138. The antibody or fragment of claim 137, wherein the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 15.

139. The antibody or fragment of claim 137, wherein the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 16.

140. The antibody or fragment of claim 137, wherein the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 17.

141. The antibody or fragment of claim 137, wherein the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

142. The antibody or fragment of claim 137, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16.

143. The antibody or fragment of claim 137, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

144. The antibody or fragment of claim 137, wherein the antibody or functional fragment thereof comprises a heavy chain comprising an amino acid sequence identical to SEQ ID NO: 15 and a light chain comprising an amino acid sequence identical to SEQ ID NO: 16.

145. The antibody or fragment of claim 137, wherein the antibody or functional fragment thereof comprises a heavy chain comprising an amino acid sequence identical to SEQ ID NO: 17 and a light chain comprising an amino acid sequence identical to SEQ ID NO: 18.

146. The antibody or fragment of claim 137, wherein the antibody or functional fragment thereof comprises a heavy chain comprising an amino acid sequence comprising the complementarity-determining regions (CDRs) of SEQ ID NO: 15 and a light chain comprising an amino acid sequence comprising the complementarity-determining regions (CDRs) of SEQ ID NO: 16.

147. The antibody or fragment of claim 137, wherein the antibody or functional fragment thereof comprises a heavy chain comprising an amino acid sequence comprising the complementarity-determining regions (CDRs) of SEQ ID NO: 17 and a light chain comprising an amino acid sequence comprising the complementarity-determining regions (CDRs) of SEQ ID NO: 18.

148. The antibody or fragment of claim 137, wherein the virus is a coronavirus.

149. The antibody or fragment of claim 148, wherein the coronavirus is a coronavirus variant.

150. The antibody or fragment of claim 148, wherein the coronavirus is a coronavirus-related virus.

151. The antibody or fragment of claim 148, wherein the coronavirus is a wildtype SARS-CoV-2 (D614G) virus.

152. The antibody or fragment of claim 148, wherein the coronavirus is a SARS-COV-2 B.1.1.7 variant virus.

153. The antibody or fragment of claim 148, wherein the coronavirus is a SARS-COV-2 B.1.351 variant virus.

154. The antibody or fragment of claim 148, wherein the coronavirus is a SARS-COV-2 P.1 variant virus.

155. The antibody or fragment of claim 148, wherein the coronavirus is a SARS-COV-2 B.1.526 variant virus.

156. The antibody or fragment of claim 148, wherein the coronavirus is a SARS-COV-2 B.1.429 variant virus.

157. The antibody or fragment of claim 148, wherein the coronavirus is a SARS-COV-2 B.1.617.1v1 variant virus.

158. The antibody or fragment of claim 148, wherein the coronavirus is a SARS-COV-2 B.1.617.2 variant virus.

159. The antibody or fragment of claim 148, wherein the coronavirus is a SARS-COV-2 B.1.1.1 variant virus.

160. The antibody or fragment of claim 148, wherein the coronavirus is a SARS-COV-2 C.37 variant virus.

161. The antibody or fragment of claim 148, wherein the coronavirus is a SARS-COV-2 Omicron (B.1.1.529) variant virus.

162. The antibody or fragment of claim 148, wherein the coronavirus is a SARS-COV-2 Omicron BA.1 (B.1.1.529.1) variant virus

163. The antibody or fragment of claim 148, wherein the coronavirus is a SARS-COV-2 Omicron BA.1.1 (B.1.1.529.1.1) variant virus.

164. The antibody or fragment of claim 148, wherein the coronavirus is a SARS-COV-2 Omicron BA.2 (B.1.1.529.2) variant virus.

165. The antibody or fragment of claim 148, wherein the coronavirus is a SARS-COV-2 B.1.621 variant virus.

166. The antibody or fragment of claim 148, wherein the coronavirus is a SARS-COV-2 WA1 virus.

167. The antibody or fragment of claim 148, wherein the coronavirus is any virus of Table 1.

168. The antibody or fragment of claim 148, wherein the coronavirus is a SARS-COV virus.

169. The antibody or fragment of claim 137, wherein the at least a portion of a virus comprises a coronavirus spike protein.

170. The antibody or fragment of claim 137, wherein the at least a portion of a virus comprises a juncture between a subdomain 1 (SD1) and a N-Terminal Domain (NTD) of a coronavirus spike protein.

171. The antibody or fragment of claim 137, wherein the at least a portion of a virus comprises a subdomain 1 (SD1) of a coronavirus spike protein.

172. The antibody or fragment of claim 137, wherein the at least a portion of a virus comprises a N-Terminal Domain (NTD) of a coronavirus spike protein.

173. A method for treating a subject having a viral infection or preventing a subject from developing a viral infection, the method comprising administering to the subject a therapeutically effective amount of a composition comprising an engineered monoclonal antibody or a functional fragment thereof, wherein the antibody or fragment thereof selectively binds at least a portion of the virus.

174. The method of claim 173, wherein the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 15.

175. The method of claim 173, wherein the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 16.

176. The method of claim 173, wherein the antibody or functional fragment thereof comprises a heavy chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 17.

177. The method of claim 173, wherein the antibody or functional fragment thereof comprises a light chain comprising an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

178. The method of claim 173, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16.

179. The method of claim 173, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

180. The method of claim 173, wherein the antibody or functional fragment thereof comprises a heavy chain comprising an amino acid sequence identical to SEQ ID NO: 15 and a light chain comprising an amino acid sequence identical to SEQ ID NO: 16.

181. The method of claim 173, wherein the antibody or functional fragment thereof comprises a heavy chain comprising an amino acid sequence identical to SEQ ID NO: 17 and a light chain comprising an amino acid sequence identical to SEQ ID NO: 18.

182. The method of claim 173, wherein the antibody or functional fragment thereof comprises a heavy chain comprising an amino acid sequence comprising the complementarity-determining regions (CDRs) of SEQ ID NO: 15 and a light chain comprising an amino acid sequence comprising the complementarity-determining regions (CDRs) of SEQ ID NO: 16.

183. The method of claim 173, wherein the antibody or functional fragment thereof comprises a heavy chain comprising an amino acid sequence comprising the complementarity-determining regions (CDRs) of SEQ ID NO: 17 and a light chain comprising an amino acid sequence comprising the complementarity-determining regions (CDRs) of SEQ ID NO: 18.

184. The method of claim 173, wherein the virus is a coronavirus.

185. The method of claim 184, wherein the coronavirus is a coronavirus variant.

186. The method of claim 184, wherein the coronavirus is a coronavirus-related virus.

187. The method of claim 184, wherein the coronavirus is a wildtype SARS-COV-2 (D614G) virus.

188. The method of claim 184, wherein the coronavirus is a SARS-COV-2 B.1.1.7 variant virus.

189. The method of claim 184, wherein the coronavirus is a SARS-COV-2 B.1.351 variant virus.

190. The method of claim 184, wherein the coronavirus is a SARS-COV-2 P.1 variant virus.

191. The method of claim 184, wherein the coronavirus is a SARS-COV-2 B.1.526 variant virus.

192. The method of claim 184, wherein the coronavirus is a SARS-COV-2 B.1.429 variant virus.

193. The method of claim 184, wherein the coronavirus is a SARS-COV-2 B.1.617.1v1 variant virus.

194. The method of claim 184, wherein the coronavirus is a SARS-COV-2 B.1.617.2 variant virus.

195. The method of claim 184, wherein the coronavirus is a SARS-COV-2 B.1.1.1 variant virus.

196. The method of claim 184, wherein the coronavirus is a SARS-COV-2 D614G virus.

197. The method of claim 184, wherein the coronavirus is a SARS-COV-2 C.37 variant virus.

198. The method of claim 184, wherein the coronavirus is a SARS-COV-2 Omicron (B.1.1.529) variant virus.

199. The method of claim 184, wherein the coronavirus is a SARS-COV-2 Omicron BA.1 (B.1.1.529.1) variant virus

200. The method of claim 184, wherein the coronavirus is a SARS-COV-2 Omicron BA.1.1 (B.1.1.529.1.1) variant virus.

201. The method of claim 184, wherein the coronavirus is a SARS-COV-2 Omicron BA.2 (B.1.1.529.2) variant virus.

202. The method of claim 184, wherein the coronavirus is a SARS-COV-2 B.1.621 variant virus.

203. The method of claim 184, wherein the coronavirus is a SARS-COV-2 WA1 virus.

204. The method of claim 184, wherein the coronavirus is any virus of Table 1.

205. The method of claim 184, wherein the coronavirus is a SARS-COV virus.

206. The method of claim 173, wherein the at least a portion of a virus comprises a coronavirus spike protein.

207. The method of claim 173, wherein the at least a portion of a virus comprises a juncture between a subdomain 1 (SD1) and a N-Terminal Domain (NTD) of a coronavirus spike protein.

208. The method of claim 173, wherein the at least a portion of a virus comprises a subdomain 1 (SD1) of a coronavirus spike protein.

209. The method of claim 173, wherein the at least a portion of a virus comprises a N-Terminal Domain (NTD) of a coronavirus spike protein.

210. A pharmaceutical composition comprising an engineered monoclonal antibody or a functional fragment thereof, wherein the antibody or fragment thereof selectively binds at least a portion of a virus.

211. The pharmaceutical composition of claim 210, wherein the antibody or fragment thereof comprises an amino acid sequence, wherein the sequence is at least 60%, 62%, 65%, 67%, 70%, 72%, 75%, 77%, 80%, 82%, 85%, 87%, 90%, 92%, 95%, 97%, 98%, 99% identical to SEQ ID NO: 1, at least 60%, 62%, 65%, 67%, 70%, 72%, 75%, 77%, 80%, 82%, 85%, 87%, 90%, 92%, 95%, 97%, 98%, 99% identical to SEQ ID NO: 2, at least 60%, 62%, 65%, 67%, 70%, 72%, 75%, 77%, 80%, 82%, 85%, 87%, 90%, 92%, 95%, 97%, 98%, 99% identical to SEQ ID NO: 3, at least 60%, 62%, 65%, 67%, 70%, 72%, 75%, 77%, 80%, 82%, 85%, 87%, 90%, 92%, 95%, 97%, 98%, 99% identical to SEQ ID NO: 4, at least 60%, 62%, 65%, 67%, 70%, 72%, 75%, 77%, 80%, 82%, 85%, 87%, 90%, 92%, 95%, 97%, 98%, 99% identical to SEQ ID NO: 5, at least 60%, 62%, 65%, 67%, 70%, 72%, 75%, 77%, 80%, 82%, 85%, 87%, 90%, 92%, 95%, 97%, 98%, 99% identical to SEQ ID NO: 6, at least 60%, 62%, 65%, 67%, 70%, 72%, 75%, 77%, 80%, 82%, 85%, 87%, 90%, 92%, 95%, 97%, 98%, 99% identical to SEQ ID NO: 7, at least 60%, 62%, 65%, 67%, 70%, 72%, 75%, 77%, 80%, 82%, 85%, 87%, 90%, 92%, 95%, 97%, 98%, 99% identical to SEQ ID NO: 8, at least 60%, 62%, 65%, 67%, 70%, 72%, 75%, 77%, 80%, 82%, 85%, 87%, 90%, 92%, 95%, 97%, 98%, 99% identical to SEQ ID NO: 9, at least 60%, 62%, 65%, 67%, 70%, 72%, 75%, 77%, 80%, 82%, 85%, 87%, 90%, 92%, 95%, 97%, 98%, 99% identical to SEQ ID NO: 10, at least 60%, 62%, 65%, 67%, 70%, 72%, 75%, 77%, 80%, 82%, 85%, 87%, 90%, 92%, 95%, 97%, 98%, 99% identical to SEQ ID NO: 11, at least 60%, 62%, 65%, 67%, 70%, 72%, 75%, 77%, 80%, 82%, 85%, 87%, 90%, 92%, 95%, 97%, 98%, 99% identical to SEQ ID NO: 12, at least 60%, 62%, 65%, 67%, 70%, 72%, 75%, 77%, 80%, 82%, 85%, 87%, 90%, 92%, 95%, 97%, 98%, 99% identical to SEQ ID NO: 13, at least 60%, 62%, 65%, 67%, 70%, 72%, 75%, 77%, 80%, 82%, 85%, 87%, 90%, 92%, 95%, 97%, 98%, 99% identical to SEQ ID NO: 14, at least 60%, 62%, 65%, 67%, 70%, 72%, 75%, 77%, 80%, 82%, 85%, 87%, 90%, 92%, 95%, 97%, 98%, 99% identical to SEQ ID NO: 15, at least 60%, 62%, 65%, 67%, 70%, 72%, 75%, 77%, 80%, 82%, 85%, 87%, 90%, 92%, 95%, 97%, 98%, 99% identical to SEQ ID NO: 16, at least 60%, 62%, 65%, 67%, 70%, 72%, 75%, 77%, 80%, 82%, 85%, 87%, 90%, 92%, 95%, 97%, 98%, 99% identical to SEQ ID NO: 17, or at least 60%, 62%, 65%, 67%, 70%, 72%, 75%, 77%, 80%, 82%, 85%, 87%, 90%, 92%, 95%, 97%, 98%, 99% identical to SEQ ID NO: 18.

212. The pharmaceutical composition of claim 210, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 1 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 2.

213. The pharmaceutical composition of claim 210, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 3 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 4.

214. The pharmaceutical composition of claim 210, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 5 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 6.

215. The pharmaceutical composition of claim 210, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 7 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 8.

216. The pharmaceutical composition of claim 210, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 9 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 10.

217. The pharmaceutical composition of claim 210, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 11 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 12.

218. The pharmaceutical composition of claim 210, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 13 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 14.

219. The pharmaceutical composition of claim 210, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 15 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 16.

220. The pharmaceutical composition of claim 210, wherein the antibody or functional fragment thereof comprises a heavy chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 17 and a light chain comprising an amino acid sequence which is at least 60%, 62%, 65%, 67%, 70%, 72%, 75%, 77%, 80%, 82%, 85%, 87%, 90%, 92%, 95%, 97%, 98%, 99% identical to SEQ ID NO: 18.

221. The pharmaceutical composition of claim 210, wherein the antibody or functional fragment thereof comprises a heavy chain comprising an amino acid sequence identical to SEQ ID NO: 1 and a light chain comprising an amino acid sequence identical to SEQ ID NO: 2.

222. The pharmaceutical composition of claim 210, wherein the antibody or functional fragment thereof comprises a heavy chain comprising an amino acid sequence identical to SEQ ID NO: 3 and a light chain comprising an amino acid sequence identical to SEQ ID NO: 4.

223. The pharmaceutical composition of claim 210, wherein the antibody or functional fragment thereof comprises a heavy chain comprising an amino acid sequence identical to SEQ ID NO: 5 and a light chain comprising an amino acid sequence identical to SEQ ID NO: 6.

224. The pharmaceutical composition of claim 210, wherein the antibody or functional fragment thereof comprises a heavy chain comprising an amino acid sequence identical to SEQ ID NO: 7 and a light chain comprising an amino acid sequence identical to SEQ ID NO: 8.

225. The pharmaceutical composition of claim 210, wherein the antibody or functional fragment thereof comprises a heavy chain comprising an amino acid sequence identical to SEQ ID NO: 9 and a light chain comprising an amino acid sequence identical to SEQ ID NO: 10.

226. The pharmaceutical composition of claim 210, wherein the antibody or functional fragment thereof comprises a heavy chain comprising an amino acid sequence identical to SEQ ID NO: 11 and a light chain comprising an amino acid sequence identical to SEQ ID NO: 12.

227. The pharmaceutical composition of claim 210, wherein the antibody or functional fragment thereof comprises a heavy chain comprising an amino acid sequence identical to SEQ ID NO: 13 and a light chain comprising an amino acid sequence identical to SEQ ID NO: 14.

228. The pharmaceutical composition of claim 210, wherein the antibody or functional fragment thereof comprises a heavy chain comprising an amino acid sequence identical to SEQ ID NO: 15 and a light chain comprising an amino acid sequence identical to SEQ ID NO: 16.

229. The pharmaceutical composition of claim 210, wherein the antibody or functional fragment thereof comprises a heavy chain comprising an amino acid sequence identical to SEQ ID NO: 17 and a light chain comprising an amino acid sequence identical to SEQ ID NO: 18.

230. The pharmaceutical composition of claim 210, wherein the antibody or functional fragment thereof comprises a heavy chain comprising an amino acid sequence comprising the complementarity-determining regions (CDRs) of SEQ ID NO: 1 and a light chain comprising an amino acid sequence comprising the complementarity-determining regions (CDRs) of SEQ ID NO: 2.

231. The pharmaceutical composition of claim 210, wherein the antibody or functional fragment thereof comprises a heavy chain comprising an amino acid sequence comprising the complementarity-determining regions (CDRs) of SEQ ID NO: 3 and a light chain comprising an amino acid sequence comprising the complementarity-determining regions (CDRs) of SEQ ID NO: 4.

232. The pharmaceutical composition of claim 210, wherein the antibody or functional fragment thereof comprises a heavy chain comprising an amino acid sequence comprising the complementarity-determining regions (CDRs) of SEQ ID NO: 5 and a light chain comprising an amino acid sequence comprising the complementarity-determining regions (CDRs) of SEQ ID NO: 6.

233. The pharmaceutical composition of claim 210, wherein the antibody or functional fragment thereof comprises a heavy chain comprising an amino acid sequence comprising the complementarity-determining regions (CDRs) of SEQ ID NO: 7 and a light chain comprising an amino acid sequence comprising the complementarity-determining regions (CDRs) of SEQ ID NO: 8.

234. The pharmaceutical composition of claim 210, wherein the antibody or functional fragment thereof comprises a heavy chain comprising an amino acid sequence comprising the complementarity-determining regions (CDRs) of SEQ ID NO: 9 and a light chain comprising an amino acid sequence comprising the complementarity-determining regions (CDRs) of SEQ ID NO: 10.

235. The pharmaceutical composition of claim 210, wherein the antibody or functional fragment thereof comprises a heavy chain comprising an amino acid sequence comprising the complementarity-determining regions (CDRs) of SEQ ID NO: 11 and a light chain comprising an amino acid sequence comprising the complementarity-determining regions (CDRs) of SEQ ID NO: 12.

236. The pharmaceutical composition of claim 210, wherein the antibody or functional fragment thereof comprises a heavy chain comprising an amino acid sequence comprising the complementarity-determining regions (CDRs) of SEQ ID NO: 13 and a light chain comprising an amino acid sequence comprising the complementarity-determining regions (CDRs) of SEQ ID NO: 14.

237. The pharmaceutical composition of claim 210, wherein the antibody or functional fragment thereof comprises a heavy chain comprising an amino acid sequence comprising the complementarity-determining regions (CDRs) of SEQ ID NO: 15 and a light chain comprising an amino acid sequence comprising the complementarity-determining regions (CDRs) of SEQ ID NO: 16.

238. The pharmaceutical composition of claim 210, wherein the antibody or functional fragment thereof comprises a heavy chain comprising an amino acid sequence comprising the complementarity-determining regions (CDRs) of SEQ ID NO: 17 and a light chain comprising an amino acid sequence comprising the complementarity-determining regions (CDRs) of SEQ ID NO: 18.

239. The pharmaceutical composition of claim 210, wherein the virus is a coronavirus.

240. The pharmaceutical composition of claim 239, wherein the coronavirus is a coronavirus variant.

241. The pharmaceutical composition of claim 239, wherein the coronavirus is a coronavirus-related virus.

242. The pharmaceutical composition of claim 239, wherein the coronavirus is a wildtype SARS-COV-2 (D614G) virus.

243. The pharmaceutical composition of claim 239, wherein the coronavirus is a SARS-CoV-2 B.1.1.7 variant virus.

244. The pharmaceutical composition of claim 239, wherein the coronavirus is a SARS-CoV-2 B.1.351 variant virus.

245. The pharmaceutical composition of claim 239, wherein the coronavirus is a SARS-CoV-2 P.1 variant virus.

246. The pharmaceutical composition of claim 239, wherein the coronavirus is a SARS-CoV-2 B.1.526 variant virus.

247. The pharmaceutical composition of claim 239, wherein the coronavirus is a SARS-CoV-2 B.1.429 variant virus.

248. The pharmaceutical composition of claim 239, wherein the coronavirus is a SARS-CoV-2 B.1.617.1v1 variant virus.

249. The pharmaceutical composition of claim 239, wherein the coronavirus is a SARS-CoV-2 B.1.617.2 variant virus.

250. The pharmaceutical composition of claim 239, wherein the coronavirus is a SARS-CoV-2 B.1.1.1 variant virus.

251. The pharmaceutical composition of claim 239, wherein the coronavirus is a SARS-CoV-2 D614G virus.

252. The pharmaceutical composition of claim 239, wherein the coronavirus is a SARS-CoV-2 C.37 variant virus.

253. The pharmaceutical composition of claim 239, wherein the coronavirus is a SARS-CoV-2 Omicron (B.1.1.529) variant virus.

254. The pharmaceutical composition of claim 239, wherein the coronavirus is a SARS-CoV-2 Omicron BA.1 (B.1.1.529.1) variant virus.

255. The pharmaceutical composition of claim 239, wherein the coronavirus is a SARS-CoV-2 Omicron BA.1.1 (B.1.1.529.1.1) variant virus.

256. The pharmaceutical composition of claim 239, wherein the coronavirus is a SARS-CoV-2 Omicron BA.2 (B.1.1.529.2) variant virus.

257. The pharmaceutical composition of claim 239, wherein the coronavirus is a SARS-CoV-2 B.1.621 variant virus.

258. The pharmaceutical composition of claim 239, wherein the coronavirus is a SARS-CoV-2 WA1 virus.

259. The pharmaceutical composition of claim 239, wherein the coronavirus is any virus of Table 1.

260. The pharmaceutical composition of claim 239, wherein the coronavirus is a SARS-CoV virus.

261. The pharmaceutical composition of claim 210, wherein the at least a portion of a virus comprises a coronavirus spike protein.

262. The pharmaceutical composition of claim 210, wherein the at least a portion of a virus comprises a juncture between a subdomain 1 (SD1) and a N-Terminal Domain (NTD) of a coronavirus spike protein.

263. The pharmaceutical composition of claim 210, wherein the at least a portion of a virus comprises a subdomain 1 (SD1) of a coronavirus spike protein.

264. The pharmaceutical composition of claim 210, wherein the at least a portion of a virus comprises a N-Terminal Domain (NTD) of a coronavirus spike protein.

265. A polynucleotide encoding an antibody or a fragment thereof, wherein the antibody or fragment thereof comprises any one of SEQ ID NOs: 1-18, or a combination thereof.

266. A vector comprising the polynucleotide of claim 265.


SEQUENCE LISTING (141 sequences)

SEQ ID NO: 1 - protein, 123 aa, synthetic construct
QMQLVQSGPEVKKPGTSVKVSCKASGFTFISSAMQWVRQARGQRLEWIGWIVVGSGNTNYAQKFQERVSITRDMSTSTAYMELSSLRSEDTAVYYCAAPYCNSTNCYDAFNIWGQGTMVTVSS

SEQ ID NO: 2 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGRSPWTFGQGTKVDIK

SEQ ID NO: 3 - protein, 130 aa, synthetic construct
QLQLQESGPGLVKPSETLSLTCTVSGGSISSSNFYWGWIRQPPGKGLEWIASITYSGRTFYNPSLKSRVAISVDTSKNQFSLKLSSVTAADTAVYYCARTFPSYYDRSGYHYLNYGMDVWGQGTTVTVSS

SEQ ID NO: 4 - protein, 110 aa, synthetic construct
NFMLTQPHSMSESPGKTVTISCTRSSGSIASNYVQWYQQRPGSSPTTVIYEDNQRPSGVPDRFSGSIDSSSNSASLTISGLKTEDEADYYCQSYDSSSWVFGGGTKLTVL

SEQ ID NO: 5 - protein, 129 aa, synthetic construct
QVQLQESGPGLVKPSQTLSLTCTVSGGSISSGGYYWSWIRQHPGKGLEWIGYIYYSGSTYYNPSLKSRVTISVDTSKNQFSLKVSSVTAADTAVYYCASSNYYDGSGYNLLESYGMDVWGQGTSVTVSS

SEQ ID NO: 6 - protein, 112 aa, synthetic construct
QSVLTQPPSVSGAPGQRVTISCTGSSSNIGAGYDVHWYQQLPGTAPKLLIYGNSNRPSGVPDRFSGSRSGTSASLAITGLQAEDEADYYCQSYDSSLSGSWVFGGGTKVTVL

SEQ ID NO: 7 - protein, 124 aa, synthetic construct
QVQLVQSGAEVKKPGSSVKVSCKASGGTFSNYAISWVRQAPGQGLEWMGGFIRIFDTAIYAQKFQGRVTITADESTSTAYMDLSSLRSEDTAVYYCARSPYYYDKSGYYYFDHWGQGTLVTVSS

SEQ ID NO: 8 - protein, 107 aa, synthetic construct
DIQMTQSPSSLSASVGDRVTITCRASQNISSYLNWYQQKPGKAPNLLIYAASSLQSGVPSRFSGSGSGTDFTLTISSLQPEDFATYYCQQSYSPQWTFGQGTKVEIK

SEQ ID NO: 9 - protein, 123 aa, synthetic construct
EVQLVQSGAEVKKPGESLKMSCKGSGYNFTFYWIGWVRQMPGKGLEWMGSIYPGDSETRYSPSFQGQVTISAATSISTAYLQWSSLKASDAAMYYCTARLSGVQTALEYFDFWGQGTLVTVSS

SEQ ID NO: 10 - protein, 107 aa, synthetic construct
DIQMTQSPSSLSASVGDRVTITCQASQDITNHLNWYQLRPGKAPKLLIYDASNLDAGVPSRFSGSGSGTDFTFTISSLQPEDIATYYCQQYDNLLLTFGGGTKVEIK

SEQ ID NO: 11 - protein, 126 aa, synthetic construct
QVQLVESGGGVVQPGRSLRLSCAASGFIFSDFGMHWVRQAPGKGLEWVAVISYDGSNKYYADSVKGRFTISRDNSKNTLYLQMNSLRAEDTAVYYCKARDYYDISGFYHPPNFDYWGQGTLVTVSS

SEQ ID NO: 12 - protein, 108 aa, synthetic construct
SYELTQPPSVSVSPGQTARIPCSGHTLPKQYAYWYQQKPGQAPVLVIYKDSERPSGIPERFSGSSSGTTVTLTISGVQAEDEANYYCQSADSSGTYWVFGGGTKLTVL

SEQ ID NO: 13 - protein, 120 aa, synthetic construct
QVQLVESGGGVVQPGRSLRLSCAASGFTFSSYDMHWVRQAPGKGLEWVAVISYDGSSKFYAESVKGRFTISRDNSKNTLYLQMNSLRAEETAVYYCVKDGEQLVPLFDYWGQGTLVTVSS

SEQ ID NO: 14 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPGVTFGPGTKVDIK

SEQ ID NO: 15 - protein, 132 aa, synthetic construct
QVQLVESGGGVVQPGRSLRLSCAASGFTFSNYGMHWVRQAPGKGLEWVAIISFDGSNRYYADSVKGRFTISRDNSKNTLYLQMDSLRAEDTAVYYCAKDLSFYYDISTGYYPQSYNSGTDVWGQGTTVTVSS

SEQ ID NO: 16 - protein, 108 aa, synthetic construct
SYELTQPPSVSVSPGQTASITCSGDKLGDKYACWYQQKPGQSPVLVIYQDNKRPSGIPERFSGSNSGNTATLTISGTQAMDEADYYCQGWDRSTGYYVFGTGTKVTVL

SEQ ID NO: 17 - protein, 133 aa, synthetic construct
QVQLVESGGGVVQPGRSLRLSCEASGFTFNTYDMHWARQAPGKGLEWVAVIWYDGSNKFYADSVKGRFTISRDNSKNTLYLQMNSLRAEDTAVYYCARDRTPVYDILTGYYWPPPRVDGMDVWGQGTTVTVSS

SEQ ID NO: 18 - protein, 112 aa, synthetic construct
DNVMTQTPFSLSVTPGQPASISCKSSQSLLHSDGKTYLYWYLQKPGQSPQLLIYEVSNRFSGVPERFSGSGSGTDFTLKISRVEAEDVGVYYCMQSIQLPFTFGQGTKLEIK

SEQ ID NO: 19 - protein, 130 aa, synthetic construct; identical to SEQ ID NO: 3

SEQ ID NO: 20 - protein, 128 aa, synthetic construct
QVQLQESGPGLVKPSETLSLTCTVSGGSVSSSNYYWSWIRQPPGKGLEWIGYMYYSGSTKYNPSLKSRVTISVDTSKNQFSLKLSSVTAADTAVYYCAREVYYYDRSGYYASDGFDIWGQGTMVTVSS

SEQ ID NO: 21 - protein, 129 aa, synthetic construct
QVQLQESGPGLVKPSETLSVTCTVSGGSISSSRYYWGWIRQPPGKGLEWIGSIYYSGSTYYNPSLKSRVTISVDTSKNQFSLKLSSVTAADTAVYYCARHAAAYYDRSGYYFIEYFQHWGQGTLVTVSS

SEQ ID NO: 22 - protein, 129 aa, synthetic construct
QVQLVQSGAEVKKPGASVKVSCKASGYTFTSYYMHWVRQAPGQGLEWMGIINSSGGSTSYAQKFQGRVTMTRDTSTSTVYMELSSLRSEDTAVYYCARPPRNYYDRSGYYQRAEYFQHWGQGTLVTVSS

SEQ ID NO: 23 - protein, 9 aa, synthetic construct
YYYDSSGYY

SEQ ID NO: 24 - protein, 15 aa, synthetic construct
CVKDGEQLVPLFDYW

SEQ ID NO: 25 - protein, 24 aa, synthetic construct
CARTFPSYYDRSGYHYLNYGMDVW

SEQ ID NO: 26 - protein, 23 aa, synthetic construct
CASSNYYDGSGYNLLESYGMDVW

SEQ ID NO: 27 - protein, 12 aa, synthetic construct [sequence omitted]

SEQ ID NO: 28 - protein, 11 aa, synthetic construct [sequence omitted]

SEQ ID NO: 29 - protein, 14 aa, synthetic construct [sequence omitted]

SEQ ID NO: 30 - protein, 34 aa, synthetic construct
QLQLQESGPGLVKPSETLSLTCTVSGGSISSSSY

SEQ ID NO: 31 - protein, 61 aa, synthetic construct
WGWIRQPPGKGLEWIGSIYYSGSTYYNPSLKSRVTISVDTSKNQFSLKLSSVTAADTAVYY

SEQ ID NO: 32 - protein, 25 aa, synthetic construct
QLQLQESGPGLVKPSETLSLTCTVS

SEQ ID NO: 33 - protein, 61 aa, synthetic construct
WGWIRQPPGKGLEWIASITYSGRTFYNPSLKSRVAISVDTSKNQFSLKLSSVTAADTAVYY

SEQ ID NO: 34 - protein, 21 aa, synthetic construct
ARTFPSYYDRSGYHYLNYGMD

SEQ ID NO: 35 - protein, 11 aa, synthetic construct
WGQGTTVTVSS

SEQ ID NO: 36 - protein, 90 aa, synthetic construct
NFMLTQPHSVSESPGKTVTISCTRSSGSIASNYVQWYQQRPGSSPTTVIYEDNQRPSGVPDRFSGSIDSSSNSASLTISGLKTEDEADYY

SEQ ID NO: 37 - protein, 32 aa, synthetic construct
NFMLTQPHSMSESPGKTVTISCTRSSGSIASN

SEQ ID NO: 38 - protein, 57 aa, synthetic construct
VQWYQQRPGSSPTTVIYEDNQRPSGVPDRFSGSIDSSSNSASLTISGLKTEDEADYY

SEQ ID NO: 39 - protein, 19 aa, synthetic construct
QSYDSSSWVFGGGTKLTVL

SEQ ID NO: 40 - protein, 10 aa, synthetic construct
  VARIANT 4: note = I or V
  VARIANT 8: note = N or R
  VARIANT 9: note = F or Y
GGSXSSSXXY

SEQ ID NO: 41 - protein, 8 aa, synthetic construct
GYTFTSYY

SEQ ID NO: 42 - protein, 23 aa, synthetic construct
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 3..7: note = This region may encompass 4-5 residues
  VARIANT 15: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
  VARIANT 21: note = Any amino acid
  VARIANT 22: note = Any amino acid
  VARIANT 23: note = Any amino acid
  VARIANT 15..23: note = This region may encompass 8-9 residues
ARXXXXXYYDRSGYXXXXXXXXX

SEQ ID NO: 43 - protein, 5 aa, synthetic construct
LYNSS

SEQ ID NO: 44 - protein, 5 aa, synthetic construct
LYNST

SEQ ID NO: 45 - protein, 5 aa, synthetic construct
LYNSA

SEQ ID NO: 46 - protein, 5 aa, synthetic construct
FYNST

SEQ ID NO: 47 - protein, 9 aa, synthetic construct
FKCYGVSPT

SEQ ID NO: 48 - protein, 9 aa, synthetic construct
FQCYGVSPT

SEQ ID NO: 49 - protein, 9 aa, synthetic construct
FKCYGVSAT

SEQ ID NO: 50 - protein, 9 aa, synthetic construct
FKCYGVSAI

SEQ ID NO: 51 - protein, 9 aa, synthetic construct
FKCYGVSPS

SEQ ID NO: 52 - protein, 9 aa, synthetic construct
FNCYGVSPS

SEQ ID NO: 53 - protein, 6 aa, synthetic construct
APAQTG

SEQ ID NO: 54 - protein, 6 aa, synthetic construct
APGQTG

SEQ ID NO: 55 - protein, 6 aa, synthetic construct
APGETG

SEQ ID NO: 56 - protein, 4 aa, synthetic construct
DDFT

SEQ ID NO: 57 - protein, 4 aa, synthetic construct
DDFL

SEQ ID NO: 58 - protein, 4 aa, synthetic construct
DDFM

SEQ ID NO: 59 - protein, 4 aa, synthetic construct
DEFT

SEQ ID NO: 60 - protein, 5 aa, synthetic construct
TSGNY

SEQ ID NO: 61 - protein, 5 aa, synthetic construct
QTGNY

SEQ ID NO: 62 - protein, 5 aa, synthetic construct
SSGNF

SEQ ID NO: 63 - protein, 5 aa, synthetic construct
STGNY

SEQ ID NO: 64 - protein, 5 aa, synthetic construct
TGGNY

SEQ ID NO: 65 - protein, 5 aa, synthetic construct
EGGNF

SEQ ID NO: 66 - protein, 5 aa, synthetic construct
VGGNY

SEQ ID NO: 67 - protein, 14 aa, synthetic construct
YNSAGGTCSSISQL

SEQ ID NO: 68 - protein, 13 aa, synthetic construct
FNPSGGTCSAEGL

SEQ ID NO: 69 - protein, 13 aa, synthetic construct
YSPGGQSCSAVGP

SEQ ID NO: 70 - protein, 13 aa, synthetic construct
YSPGGQSCSAIGP

SEQ ID NO: 71 - protein, 13 aa, synthetic construct
FSPDGKPCTPPAF

SEQ ID NO: 72 - protein, 13 aa, synthetic construct
FSPDGKPCTPPAL

SEQ ID NO: 73 - protein, 14 aa, synthetic construct
YQAGSTPCNGQVGL

SEQ ID NO: 74 - protein, 14 aa, synthetic construct
YQAGSKPCNGQTGL

SEQ ID NO: 75 - protein, 14 aa, synthetic construct
YQAGSTPCNGVEGF

SEQ ID NO: 76 - protein, 188 aa, synthetic construct
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSF

SEQ ID NO: 77 - protein, 188 aa, synthetic construct
RFPNITNLCPFGEVFNATTFASVYAWNRKRISNCVADYSVLYNSTSFSTFKCYGVSPTKLNDLCFTNVYADSFVVRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFHPTNGVGYQPYRVVVLSF

SEQ ID NO: 78 - protein, 188 aa, synthetic construct
RFPNITNLCPFGEVFNATTFASVYAWNRKRISNCVADYSVLYNSTSFSTFKCYGVSPTKLNDLCFTNVYADSFVVRGDEVRQIAPGQTGKIADYNYKLPDDFTGCVIAWNSNNLDSKVGGNYNYLYRLFRKSNLKPFERDISTEIYQAGSTPCNGVEGFNCYFPLKSYGFHPTNGVGYQPYRVVVLSF

SEQ ID NO: 79 - protein, 169 aa, synthetic construct
RFPNITNFCPFDKVFNATRFPNVYAWQRTKISDCIADYTVLYNSTSFSTFKCYGVSPSKLIDLCFTSVYADTFLIRFSEVRQIAPGETGVIADYNYKLPDDFTGCVLAWNTAQQDIGSYFYRSHRAVKLKPFERDLSSDENGVRTLSTYDFNPNVPLDYQATRVVVLSF

SEQ ID NO: 80 - protein, 25 aa, synthetic construct
AKDLSFYYDISTGYYPQSYNSGTDV

SEQ ID NO: 81 - protein, 11 aa, synthetic construct
QGWDRSTGYYV

SEQ ID NO: 82 - protein, 26 aa, synthetic construct
ARDRTPVYDILTGYYWPPPRVDGMDV

SEQ ID NO: 83 - protein, 9 aa, synthetic construct
MQSIQLPFT

SEQ ID NO: 84 - protein, 96 aa, synthetic construct
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYC

SEQ ID NO: 85 - protein, 87 aa, synthetic construct
SYELTQPPSVSVSPGQTASITCSGDKLGDKYACWYQQKPGQSPVLVIYQDSKRPSGIPERFSGSNSGNTATLTISGTQAMDEADYYC

SEQ ID NO: 86 - protein, 96 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYC

SEQ ID NO: 87 - protein, 93 aa, synthetic construct
DIVMTQTPLSLSVTPGQPASISCKSSQSLLHSDGKTYLYWYLQKPGQSPQLLIYEVSSRFSGVPDRFSGSGSGTDFTLKISRVEAEDVGVYYC

SEQ ID NO: 88 - DNA, 11 bp, synthetic construct
  source 1..11: mol_type = other DNA
tgtgcgaaaga

SEQ ID NO: 89 - DNA, 31 bp, synthetic construct
  source 1..31: mol_type = other DNA
gtattacgatattttgactggttattataac

SEQ ID NO: 90 - DNA, 32 bp, synthetic construct
  source 1..32: mol_type = other DNA
attactactactactacggtatggacgtctgg

SEQ ID NO: 91 - protein, 10 aa, synthetic construct
YYDILTGYYN

SEQ ID NO: 92 - protein, 10 aa, synthetic construct
YYYYYGMDVW

SEQ ID NO: 93 - DNA, 51 bp, synthetic construct
  source 1..51: mol_type = other DNA
tgtgcgaaagatctatctttctactacgatatttcgactggatattatccc

SEQ ID NO: 94 - DNA, 29 bp, synthetic construct
  source 1..29: mol_type = other DNA
agtcctacaactccggtacggacgtctgg

SEQ ID NO: 95 - protein, 17 aa, synthetic construct
CAKDLSFYYDISTGYYP

SEQ ID NO: 96 - protein, 10 aa, synthetic construct
QSYNSGTDVW

SEQ ID NO: 97 - DNA, 11 bp, synthetic construct
  source 1..11: mol_type = other DNA
tgtgcgagaga

SEQ ID NO: 98 - DNA, 84 bp, synthetic construct
  source 1..84: mol_type = other DNA
tgtgcgagagatagaacgcccgtttacgatattttgactggttattattggccccctccgagggtcgacggtatggacgtctgg

SEQ ID NO: 99 - protein, 28 aa, synthetic construct
CARDRTPVYDILTGYYWPPPRVDGMDVW

SEQ ID NO: 100 - protein, 4 aa, synthetic construct
RTPV

SEQ ID NO: 101 - protein, 8 aa, synthetic construct
GFTFISSA

SEQ ID NO: 102 - protein, 8 aa, synthetic construct
GFTFSSYD

SEQ ID NO: 103 - protein, 10 aa, synthetic construct
GGSISSSNFY

SEQ ID NO: 104 - protein, 10 aa, synthetic construct
GGSISSGGYY

SEQ ID NO: 105 - protein, 8 aa, synthetic construct
GGTFSNYA

SEQ ID NO: 106 - protein, 8 aa, synthetic construct
GYNFTFYW

SEQ ID NO: 107 - protein, 8 aa, synthetic construct
GFIFSDFG

SEQ ID NO: 108 - protein, 8 aa, synthetic construct
GFTFSNYG

SEQ ID NO: 109 - protein, 8 aa, synthetic construct
GFTFNTYD

SEQ ID NO: 110 - protein, 8 aa, synthetic construct
IVVGSGNT

SEQ ID NO: 111 - protein, 8 aa, synthetic construct
ISYDGSSK

SEQ ID NO: 112 - protein, 7 aa, synthetic construct
ITYSGRT

SEQ ID NO: 113 - protein, 7 aa, synthetic construct
IYYSGST

SEQ ID NO: 114 - protein, 8 aa, synthetic construct
FIRIFDTA

SEQ ID NO: 115 - protein, 7 aa, synthetic construct
IYPGDSE

SEQ ID NO: 116 - protein, 7 aa, synthetic construct
ISYDGSN

SEQ ID NO: 117 - protein, 8 aa, synthetic construct
ISFDGSNR

SEQ ID NO: 118 - protein, 8 aa, synthetic construct
IWYDGSNK

SEQ ID NO: 119 - protein, 16 aa, synthetic construct
AAPYCNSTNCYDAFNI

SEQ ID NO: 120 - protein, 13 aa, synthetic construct
VKDGEQLVPLFDY

SEQ ID NO: 121 - protein, 22 aa, synthetic construct
ARTFPSYYDRSGYHYLNYGMDV

SEQ ID NO: 122 - protein, 21 aa, synthetic construct
ASSNYYDGSGYNLLESYGMDV

SEQ ID NO: 123 - protein, 17 aa, synthetic construct
ARSPYYYDKSGYYYFDH

SEQ ID NO: 124 - protein, 16 aa, synthetic construct
TARLSGVQTALEYFDF

SEQ ID NO: 125 - protein, 19 aa, synthetic construct
KARDYYDISGFYHPPNFDY

SEQ ID NO: 126 - protein, 7 aa, synthetic construct
QSVSSSY

SEQ ID NO: 127 - protein, 6 aa, synthetic construct
QSISSY

SEQ ID NO: 128 - protein, 8 aa, synthetic construct
SGSIASNY

SEQ ID NO: 129 - protein, 9 aa, synthetic construct
SSNIGAGYD

SEQ ID NO: 130 - protein, 6 aa, synthetic construct
QNISSY

SEQ ID NO: 131 - protein, 6 aa, synthetic construct
QDITNH

SEQ ID NO: 132 - protein, 6 aa, synthetic construct
TLPKQY

SEQ ID NO: 133 - protein, 6 aa, synthetic construct
KLGDKY

SEQ ID NO: 134 - protein, 11 aa, synthetic construct
QSLLHSDGKTY

SEQ ID NO: 135 - protein, 9 aa, synthetic construct
QQYGRSPWT

SEQ ID NO: 136 - protein, 10 aa, synthetic construct
QQSYSTPGVT

SEQ ID NO: 137 - protein, 9 aa, synthetic construct
QSYDSSSWV

SEQ ID NO: 138 - protein, 12 aa, synthetic construct
QSYDSSLSGSWV

SEQ ID NO: 139 - protein, 9 aa, synthetic construct
QQSYSPQWT

SEQ ID NO: 140 - protein, 9 aa, synthetic construct
QQYDNLLLT

SEQ ID NO: 141 - protein, 11 aa, synthetic construct
QSADSSGTYWV